SEQUENCE LISTING (235 sequences)

SEQ ID NO: 1 - protein, 286 aa, Synechococcus elongatus
MTLAIRPKLAFNWPTALFMVAIHIGALLAFLPANFNWPAVGVMVAFTLYYITGCFGITLGWHRLISHRSFEVPKWLEYVLVFCGTLAMQHGPIEWIGLHRHHHLHSFTDQDVDHHDSNKGFLWSHFLWMIYEIPARTEVDKFTRDIAGDPVYRFFNKYFFGVQVLLGFTVLLYAWGEAWVGNGWSFVVWGIFARLVVVYHVTWLVNSATHKFGYRSHESGDQSTNCWWFTVALLAFGEGWHNNHHAYQYSARHGLQWWEFDLTWLIICGLKKVGLARKIKVASPNN

SEQ ID NO: 2 - DNA, 834 bp, Synechococcus elongatus
atgacccttgctatccgacccaagcttgccttcaactggccgaccgccctgttcatggtcgccattcacattggagcactgttagcgttcctgccggccaactttaactggcccgctgtgggcgtgatggttgcgctgtattacattaccggttgttttggcatcaccctaggctggcaccggctaatttcgcaccgtagctttgaagttcccaaatggctggaatacgtgctggtgttctgtggcaccttggccatgcagcacggcccgatcgaatggatcggtctgcaccgccaccatcacctccactctgaccaagatgtcgatcaccacgactccaacaagggtttcctctggagtcacttcctgtggatgatctacgaaattccggcccgtacggaagtagacaagttcacgcgcgatatcgctggcgaccctgtctatcgcttctttaacaaatatttcttcggtgtccaagtcctactgggggtacttttgtacgcctggggcgaggcttgggttggcaatggctggtctttcgtcgtttgggggatcttcgcccgcttggtggtggtctaccacgtcacttggctggtgaacagtgctacccacaagtttggctaccgctcccatgagtctggcgaccagtccaccaactgctggtgggttgcccttctggcctttggtgaaggctggcacaacaaccaccacgcctaccagtactcggcacgtcatggcctgcagtggtgggaatttgacttgacttggttgatcatctgcggcctgaagaaggtgggtctggctcgcaagatcaaagtggcgtctccaaacaac

SEQ ID NO: 3 - protein, 284 aa, Artificial Sequence
  Synthetic codon optimized DSG with a C-terminal ER retention sequence
MTLAIRPKLAFNWPTALFMVAIHIGALLAFLPANFNWPAVGVMVALYYITGCFGITLGWHRLISHRSFEVPKWLEYVLVFCGTLAMQHGPIEWIGLHRHHHLHSDQDVDHHDSNKGFLWSHFLWMIYEIPARTEVDKFTRDIAGDPVYRFFNKYFFGVQVLLGVLLYAWGEAWVGNGWSFVVWGIFARLVVVYHVTWLVNSATHKFGYRSHESGDQSTNCWWVALLAFGEGWHNNHHAYQYSARHGLQWWEFDLTWLIICGLKKVGLARKIKVASPNNGKSKIN

SEQ ID NO: 4 - DNA, 855 bp, Artificial Sequence
  Synthetic exemplary codon optimized DSG sequence with a stop codon
atgacactcgccataaggccaaaactggctttcaattggcctactgctctcttcatggttgctatccacattggagctctgttggcttttcttcctgctaacttcaactggccagcagttggagttatggttgcgctttactacatcacgggatgctttggaatcacactaggttggcatcgactcatctcacatcgtagtttcgaggttcctaagtggctagagtacgttctcgttttctgtggaacgcttgcaatgcaacatggacctattgagtggatcggtcttcacagacaccatcatttgcactctgatcaggacgttgatcaccatgatagcaacaagggattcctatggagccacttcctatggatgatctatgagatccctgctagaacagaggtggacaagttcactagagatatcgctggagatcccgtctaccgtttcttcaacaagtacttcttcggagtccaggttctacttggtgttcttctatacgcttggggtgaagcatgggttggtaatggatggtcctttgttgtctggggaatctttgctagactggtcgttgtctaccatgttacctggcttgtgaactctgctacccataagttcggataccgttcacatgagtctggagaccaatccactaactgttggtgggttgctttacttgcattcggagagggttggcataacaatcatcacgcttaccagtactctgcaagacacggtttacaatggtgggagtttgacttgacctggctaatcatctgcggacttaagaaggtcggtcttgctaggaagatcaaagtggcttccccaaacaacgggaaatcgaagatcaactga

SEQ ID NO: 5 - protein, 6 aa, Artificial Sequence
  ER retention sequence
GKSKIN

SEQ ID NO: 6 - protein, 4 aa, Artificial Sequence
  Synthetic retention sequence
KDEL

SEQ ID NO: 7 - protein, 4 aa, Artificial Sequence
  Synthetic retention sequence
HDEL

SEQ ID NO: 8 - DNA, 43 bp, Artificial Sequence
  Synthetic primer
tctagagaattcaaaaaaatgacactcgccataaggccaaaac

SEQ ID NO: 9 - DNA, 41 bp, Artificial Sequence
  Synthetic primer
ctcgagggatccctaattaatcagttgatcttcgatttccc

SEQ ID NO: 10 - DNA, 42 bp, Artificial Sequence
  Synthetic primer
tctagagaattcaaaaaaatgacccttgctatccgacccaag

SEQ ID NO: 11 - DNA, 41 bp, Artificial Sequence
  Synthetic primer
ctcgagggatcctcaactaattaattgattttagattttcc

SEQ ID NO: 12 - DNA, 24 bp, Artificial Sequence
  Synthetic primer
atttcaagctataccaagcataca

SEQ ID NO: 13 - DNA, 25 bp, Artificial Sequence
  Synthetic primer
caaccttgattggagacttgaccaa

SEQ ID NO: 14 - DNA, 40 bp, Artificial Sequence
  Synthetic primer
taaaccgtgtcttgcagagtannngtaagcgtgatgattg

SEQ ID NO: 15 - DNA, 25 bp, Artificial Sequence
  Synthetic primer
tactctgcaagacacggtttacaat

SEQ ID NO: 16 - DNA, 40 bp, Artificial Sequence
  Synthetic primer
taaaccgtgtcttgcagagtangggtaagcgtgatgattg

SEQ ID NO: 17 - DNA, 40 bp, Artificial Sequence
  Synthetic primer
gtactctagccacttaggaacnnngaaactacgatgtgag

SEQ ID NO: 18 - DNA, 27 bp, Artificial Sequence
  Synthetic primer
gttcctaagtggctagagtacgttctc

SEQ ID NO: 19 - DNA, 36 bp, Artificial Sequence
  Synthetic primer
gtccacctctgttctagcaggnnnctcatagatcat

SEQ ID NO: 20 - DNA, 24 bp, Artificial Sequence
  Synthetic primer
cctgctagaacagaggtggacaag

SEQ ID NO: 21 - DNA, 36 bp, Artificial Sequence
  Synthetic primer
acagttagtggattggtctccnnnctcatgtgaacg

SEQ ID NO: 22 - DNA, 24 bp, Artificial Sequence
  Synthetic primer
ggagaccaatccactaactgttgg

SEQ ID NO: 23 - DNA, 39 bp, Artificial Sequence
  Synthetic primer
cttgtccacctctgttctmnnaggmnnctcatagatcat

SEQ ID NO: 24 - DNA, 30 bp, Artificial Sequence
  Synthetic primer
agaacagaggtggacaagttcactagagat

SEQ ID NO: 25 - DNA, 36 bp, Artificial Sequence
  Synthetic primer
acagttagtggattggtcmnnmnnctcatgtgaacg

SEQ ID NO: 26 - DNA, 28 bp, Artificial Sequence
  Synthetic primer
gaccaatccactaactgttggtgggttg

SEQ ID NO: 27 - DNA, 40 bp, Artificial Sequence
  Synthetic primer
gtactctagccacttaggaacgcggaaactacgatgtgag

SEQ ID NO: 28 - DNA, 27 bp, Artificial Sequence
  Synthetic primer
gttcctaagtggctagagtacgttctc

SEQ ID NO: 29 - DNA, 21 bp, Artificial Sequence
  Synthetic primer
accgtgtcttgcagagtactg

SEQ ID NO: 30 - DNA, 21 bp, Artificial Sequence
  Synthetic primer
cagtactctgcaagacacggt

SEQ ID NO: 31 - DNA, 35 bp, Artificial Sequence
  Synthetic primer
tctagagaattcaaaatgccaacttctggaactac

SEQ ID NO: 32 - DNA, 37 bp, Artificial Sequence
  Synthetic primer
ctcgagggatccttaaaagaacttaccagtttcgtag

SEQ ID NO: 33 - protein, 278 aa, Synechococcus elongatus
MTLAIRPKLAFNWPTALFMVAIHIGALLAFLPANFNWPAVGVMVALYYITGCFGITLGWHRLISHRSFEVPKWLEYVLVFCGTLAMQHGPIEWIGLHRHHHLHSDQDVDHHDSNKGFLWSHFLWMIYEIPARTEVDKFTRDIAGDPVYRFFNKYFFGVQVLLGVLLYAWGEAWVGNGWSFVVWGIFARLVVVYHVTWLVNSATHKFGYRSHESGDQSTNCWWVALLAFGEGWHNNHHAYQYSARHGLQWWEFDLTWLIICGLKKVGLARKIKVASPNN

SEQ ID NO: 34 - protein, 278 aa, Synechococcus elongatus; identical to SEQ ID NO: 33

SEQ ID NO: 35 - protein, 270 aa, Synechococcus sp.
MTIARSSDRPLNWPIILFMVAVHFGALFALLPSNFSWSAIGVAFILYFITGCLGITLGWHRLISHRSFRVPQWLEYFFLFCGSLSCQSGAIEWIGLHRHHHAFSDQEADHHNSGRGFWWSHMGWLLHDVPAKSEIRKFTKDINTDPVYILVDKYFLVLQIALGALLYALGGWSFVVWGIFVRLVVVYHFTWFVNSATHKFGYRTYESGDRSTNCWWVALCTFGEGWHNNHHAYQYSARHGLRWWEIDVTWMIIRSLQAVGLADKVKLAKP

SEQ ID NO: 36 - protein, 310 aa, Synechococcus sp.
MLSRACVYHVGQWKKYGASAYHVPQYNFQKGYFMTVATSQKLPLDWTTIIYFSLIHLVALLAFLPGNFSWQAVGVFLLFHWITGGIGITLGFHRLVSHRSFEVPKWLEYFLIFCGTLACQGGPIDWIGLHRIHHKHSDNTPDPHDSNKGFWWSHIGWMLFEIPARGDIDRYIKDIKDDPFYNFCQKYMVLIQVALGLALYAWGEAWVGNGWSFVIWGVFLRLAVVFHCTWFVNSATHKFGYKSHESNDHSKNCWWVALVTYGEGWHNNHHAYQYSARHGLNWWEIDMTWMTIRFLQALGLAKNIRLAPAK

SEQ ID NO: 37 - protein, 272 aa, Geitlerinema sp.
MTLATPTKLSLNWPVITFMAVIHIGALFALLPSNFSWAAVGVALFLHWVTGGLGITLGLHRLVAHRSFQTPKWLEYFLVFCASLAAQGGPIEWVGLHRHHHLHSDQSVDHHNSGKGFWWSHMGWMLYEVPAKKEIHRFIKDIAEDPVYLFFDKYFLLVQAALGVVLYFLGGWPFVVWGVFVRLVLVYHCTWFVNSATHKFGYRTYDAGDRSTNCWWVALVTYGEGWHNNHHAFQYSARHGLRWWEIDMTWMTIRLLQMLGLATKVKLINNEQ

SEQ ID NO: 38 - protein, 279 aa, Thermosynechococcus elongatus
MFMTQATVAKPPIAWPTATFIIFVHLGALLAFLPSMFSWQAVLLALVLHWLTAGIGITLGWHRLVSHRSFQVPKWLEYFLVFCGTLSMQGGPIWWVGLHRHHHLYSDQPNDHHDSRKGFWWSHIEWMFHEVPAEAEIPRFTKDIADDPVYRFLDRYFLPIQVVLAIVLYLWGGWPFVVWGIFVRLVTVYHTTWLVNSATHTFGYRTFETTDHSTNCWWVALLTFGEGWHNNHHAYQYSARHGLQWWELDLTWLTIRFLQLLGLAKKVRLVEAPAASCQD

SEQ ID NO: 39 - protein, 275 aa, Oscillatoriales cyanobacterium
MTVATSNNKLKHDWAVILFMVFIHVAALLAFLPANFSWVAVGLAVFLHWVTGGLGITLGWHRMLTHRSFQAPKWLEYFLVFCGTLSMQGGPIWWVGLHRHHHLYSDQNNDHHDSNKGFWWSHMGWMLYDVPAEQEAERFTKDIADDRFYQFLDQYFFPIQVAFAALLYLIGGWSFVVWGIFVRLVVVFHCTWLVNSATHKFGYRTYEADDRSTNCWWVALLTYGEGWHNNHHAFQYSARHGMQWWEIDLTWMTIQLLQMLGLATKVKLVEETEVS

SEQ ID NO: 40 - protein, 279 aa, Leptolyngbya sp.
MTVANVPKLPINWPTAIFMVAVHSIALLAFLPSNFSWSAIAVALFLHWVTGCLGITLGWHRLVAHRSFTVPKWLEYFFVFCGSLACQHGPIEWIGLHRHHHAHSDQPVDHHSSTKGFWWSHMGWIFYEVPADGDVPRLTKDIARDPFYLFCENYFLLMQIALGLLLYVIGGWSFVLWGIFVRLVAVYHCTWFVNSATHKFGYKTFESNDYSTNCWWVAVVTYGEGWHNNHHAFQFSARHGLKWWEIDATWMTIQLLSKLGLATKVKIPKQEDVQAALLS

SEQ ID NO: 41 - protein, 272 aa, Leptolyngbya sp.
MTVATPNVQPRINWPTAVFMVLIHALTLLALIPGNTTWGAVGIALALHWITGCLGITLGWHRLIAHRSFQVPKWLEYFFVICGSLACQHGPIEWVGLHRHHHRFSDQEADHHNSGRGFWWSHMGWIFHEVPSQAEVYRFTRDISSDPVYQFCNRYFLPMQLPLAIGLYLMGGWPFVVWGIFVRLVVVYHCTWLVNSATHKFGYRSHETDDASTNCWWVAVLTYGEGWHNNHHAFQHSARHGLQWWEIDLTWMHIRLLKALGLASKIKLASDA

SEQ ID NO: 42 - protein, 277 aa, Prochlorothrix hollandica
MTVATAEKRPLEWTTILFILGLHLGALCVLFPSNFSWYAVALALFMHWFTGCLGITLGWHRLISHRSFQVPKWLEYFFVFCGSLSCQSGPIEWIGLHRHHHAYSDQEEDHHNSNKGFWWSHMNWMWVDVPARKQLPRFTRDIASDPVYLFFDKYFIPLQFAVGIIFFIASDAIVGNGWSFVVWGVFLRIVLLYHCTWFVNSATHKFGYRTHETTDNSRNCWWVALLTYGEGWHNNHHAFQYSARHGWQWWEIDITWLTIRLLERLGLATKVKLISEA

SEQ ID NO: 43 - protein, 272 aa, Nodularia spumigena
MTIATSTKLQINWVNTLFFLALHIGALFAFLPSNFSWNAVGVALLLYWITGGLGITLGFHRLVTHRSFQTPKWLEYFLVFCGTLACQGGPIEWIGTHRIHHVHSDTDPDPHDSNKGFWWSHIGWLIYHSPSHADVPRFTKDIAEDPVYQFLQKYFILVQVALGLLLMYLGGWSFVVWGVFARIVWVYHCTWLVNSATHKFGYRSYDSGDRSTNCWWVAVLVFGEGWHNNHHAFQYSARHGLEWWEIDLTWMTVQLLQSLGLATNVKLAAKKQ

SEQ ID NO: 44 - protein, 272 aa, Nostoc azollae
MTIATSTKPQINWVNTLFFIALHIGALFAFFPGNFSWTAVGVALFLYWVSGGLGITLGFHRLVTHRSFQTPKWLEYFLVFCGTLACQGGPIEWVGTHRIHHLHSDTEQDPHDSNKGFWWSHIGWLIFHSPAHSQIPRFTKDIAGDPVYQFFQKYFILIQLVLGAVLLFLGGWSFVVWGVFVRIVWVYHCTWLVNSATHKFGYQSHDSGDRSTNCWWVAVLVFGEGWHNNHHAYQYSARHGLEWWEIDMTWMTIQLLQLFGLATNVKLAERKA

SEQ ID NO: 45 - protein, 274 aa, Synechocystis sp.
MTIATSTKPTLNWVNITFFAALHIGALFALLPSNFNWRAVGVAFFLYWVTGGLGITLGFHRLVTHRSFQTPKWLEYFLALCGTLACQGGPFEWIGMHRIHHLHSDQDLDPHDSNKGFWWSHLGWMIHHAPAQDEISRYIKDISDDPVYQFYQKNMIGIQVALGVILLLLGGWSFVVWGIFARVVVVYHCTWLVNSATHKFGYRSHESGDNSTNCWWVALLVFGEGWHNNHHAFQYSARHGLQWWEFDLTWLTIRLLQTLGLATNVKLAAIPVKE

SEQ ID NO: 46 - protein, 275 aa, Chamaesiphon minutus
MTVAASTKLRLDWTMIIALVIFHIGAICAFLPGLFSWKAVGVALLLHWITGGLGITLGWHRLLSHRSFQVPKWLEYFFAFCGTLALQGGILWWVGLHRHHHLHSDQDVDHHDSKKGFLWSHVRWMCFEVPAESDIPRFTKDIADDRFYQFLNKYFFPLQVVLGVVLYAIGGWPFVFWGVFARLVIVYHCTWLVNSATHKFGYRNFETTDNSTNCWWVAITTYGEGWHNNHHAYQYSARHGMKWWEIDITWMTIQVLQFLGLATKVKLVEAGTAKD

SEQ ID NO: 47 - protein, 270 aa, Chroococcidiopsis thermalis
MTIATPTKLTINWVNTLFFIGLHFGALFALLPSNFSWQAVGVAVFLYWVTGGLGVTLGFHRLVTHRSFQTPKWLEYFLVFCGTLACQGGPIEWVGTHRAHHLYSDTEPDPHDSNKGFWWSHIGWLIYRRPIELDLARFTKDIAGDPVYQFLEKNMIFIQIALGVVLLLLGGWSFVVWGIFVRVVFVYHCTWLVNSATHKFGYRSHESGDRSTNCWWVALLVFGEGWHNNHHAFQYSARHGLEWWEVDLTWMTICLLQALGLAKNVKLAEK

SEQ ID NO: 48 - protein, 272 aa, Cyanothece sp.
MTIATTATPLRLNWAVILFMVAIHIGALFVLLPSNFSWTAVGLALLLHWITGGLGITLAWHRLVTHRSFQTPKWLEYLLVFCGVLSCEGGVIEWVGLHRHHHMHSDQEVDHHNANRGFWWSHMGWMLYEVPAKSEIERLTKDINTDPVYVFLDKYFLPIQVVFGVLLYLLGGWPFVVWGIFFRLVLVYHCTWFVNSATHQFGYRTYESGDRSTNCWWVALVTYGEGWHNNHHAYPHSARHGMKWWEIDTTWIMIQVLRFLGLATKVRLVEGN

SEQ ID NO: 49 - protein, 272 aa, Nostoc sp. PCC7524
MTIATSTKPQINWVNTLFFLGLHIGALFAFVPGNFSWAAVGVAFFLYWVTGGLGITLGFHRLVTHRSFQTPKWLEYILVFCGTLACQGGPIEWVGTHRIHHLHSDTEPDPHDSNKGFWWSHIGWLIYHSPAHADVPRFTKDIAEDPVYQFLQKYFIFIQIALGLLLLYLGGWSFVVWGIFARIVWVYHCTWLVNSATHKFGYRSHDSGDNSTNCWWVAVLVFGEGWHNNHHAFQYSARHGLEWWEIDLTWMTIQLLQVLGLATNVKLADKKQ

SEQ ID NO: 50 - protein, 270 aa, Dactylococcopsis salina
MTVATEKKLPFNWPIFAYIAAIHLVALLAFFPNFFSWSAVGVALIMHWVTGGLGVTLGFHRLLSHRSLEVPKWLEYFLVFCGTLSAQGSVIDWVGLHRMHHKYSDKNPDPHDSNKGFLWSHIGWMLHEIPLKEEIPKYTKDVSSDPVYQFFSKYFILIQVAFGLFLYAIGGWSFVIWGVFVRLAVVFHCTWFVNSATHKFGYKTYESQDESRNCWWVALLTYGEGWHNNHHAYQYSARHGLQWWELDITWMMIRSLEILGLAKNVKLIKE

SEQ ID NO: 51 - protein, 270 aa, Oscillatoria nigro-viridis
MTIATSKKTSPDWPVIGFMAFLHIGALFALLPGNFSWSAVGLAVFLHWVTGGLGITLGFHRLVTHRSFQTPKWLEYFLVICGTLSCEGGPIDWVGMHRLHHIHSDTETDPHDSNQGFWWSHMGWMLSDLPIRVEIPRFTKDIGDDPVYKFLQAYFIPIQVVFGVLLYLLGGWSFVVWGVFMRIVVVFHCTWLVNSATHKFGYRTYESGDRSTNCWWVALLTYGEGWHNNHHAFQYSARHGLQWWEIDFTWMTIQLLQSLGLATKVKLAEK

SEQ ID NO: 52 - protein, 270 aa, Microcoleus vaginatus
MTIATSKKTSPDWPVVGFMAFLHIGALFALLPGNFSWSAVGLAVFLHWVTGGLGITLGFHRLVTHRSFQTPKWLEYFLVICGTLSCEGGPIDWVGMHRLHHIHSDTETDPHDSNQGFWWSHMGWMLSDLPIRVEIPRFTKDIGDDPVYKFLQAYFIPIQVVFGVLLYLLGGWSFVVWGVFMRIVVVFHCTWLVNSATHKFGYRTYESGDGSTNCWWVALLTYGEGWHNNHHAFQYSARHGLQWWEIDFTWMTIQLLQALGLATKVKLAEK

SEQ ID NO: 53 - protein, 271 aa, Leptolyngbya sp.
MTVATSQKLSLDWTTVIYFSLIHIVALLAFLPGNFSWQAVGVFLLFHWITGGVGITLGFHRLVSHRSFDVPKWVEYILIFCGTLACQGGPLDWIGLHRIHHKHSDTTPDPHDSNKGFWWSHIGWMLFEIPARADIDRYIKDIKDDPFYNFCQNYMIPIQVVVGLLLFAWGGWSFVTWGIFMRLAVVFHCTWFVNSATHKFGYQSHESNDKSRNCWWVALVTYGEGWHNNHHAYQYSARHGLNWWEVDMTWMTIRLLQILGLAKNIKLAPTD

SEQ ID NO: 54 - protein, 272 aa, Oscillatoria sp.
MTIATSTKLSPDWTIIGFMAFLHIGALFALVPSNFSWPAIALALFMHWVTGGLGVTLAFHRLVTHRSFQTPKWLEYFLVFCGTLSCQGGPIEWVGMHRLHHQHSDTEPDPHDSNQGFWWSHMGWMLYKLPIRDQIPRYTKDIADDPVYKFFQSYFIPIQVAFGILLYFLGGWPFVVWGVFARIVIVFHCTWLVNSATHKFGYRTYDAGDRSTNCWWVAVLTYGEGWHNNHHAFQYSARHGLKWWEIDLTWMTIKFLQTLGLATKVKLAEAEN

SEQ ID NO: 55 - protein, 272 aa, Cylindrospermum stagnale
MTIATSTKPQLNWVNTLFFAALHIGALFALLPSNFSWNAVGVALLLYWITGGLGITLGFHRLVTHRSFQTPKWLEYLLVLFGTLSCQGGPIEWVGIHRLHHLHSDTGSDPHDSNKGFWWSHMGWLVYHSPAQADVPRFTKDISEDPVYQFLQKYFILIQVALGIGLLLLGGWSFVVWGIFARIVWVYHCTWLVNSATHKFGYRTHDSGDHSTNCWWVAVLVFGEGWHNNHHAFQYSARHGLEWWEIDLTWMTIQVLQLLGLATNVKLAERKQ

SEQ ID NO: 56 - protein, 272 aa, Calothrix sp.
MTIANSTKPQINWVNTPFFIFLHLGALFALVPSNFSWNAVGVALFLYWVTGGLGITLGYHRLVTHRSFQTPKWLEYVLVFFGTLACQGGPIEWVGTHRMHHLHSDTEKDPHDSNQGFWWSHMGWLIYNSPTQPEIPRFTKDIVDDPVYQFLQKYFILIQFALAALLLALGGWSFVVWGVFVRIIWVYHCTWLVNSATHKFGYRTHESGDRSTNCWWVAVLVFGEGWHNNHHAFQYSARHGLEWWEIDLTWMTIQLLQALGLATNVKLADKKS

SEQ ID NO: 57 - protein, 277 aa, Microcystis aeruginosa
MTVATSEKLPIDWVTLIYMAFIHLVALLAFLPSNFSWGAVGVTLILYWITGGLGITLGFHRLVSHRSFKTPKWLEYFLVFCGTLACQGGAIAWIGLHRIHHKYSDTAPDPHDSNKGFWWSHMGWMLHEIPADEEIARFTQDIADDPFYKFCQNYFVPIQIVLGLILYAMGGWPFVIWGIFVRLVLVFHSTWFVNSATHKFGYVSHESHDNSKNCWWVALLTFGEGWHNNHHAYQYSARHGLQWWEVDLTWMTIRLLQILGLATNIKLPPTTATATES

SEQ ID NO: 58 - protein, 277 aa, Microcystis aeruginosa; identical to SEQ ID NO: 57

SEQ ID NO: 59 - protein, 282 aa, Thermosynechococcus elongatus
MFESFMTSVSSLPSRPLRPNWGVIFFMAIVHLGALLVFVPGTFSWSAVLLCFVLYWVSGGLGITLGWHRLVTHRSFQCPKWLEYFFVFCGTLACEGGIIEWVGLHRNHHLHSDQELDQHNSQKGFWWSHMGWMLQEVPAKAEVERLTKDINTDPVYRFLNQQFLPIQGVLGVLLYLWGGLPFVVWGIFVRLVLVYHLTWFVNSATHKFGYRTFESGDRSTNCWWVALLTFGEGWHNNHHTYPHSARHGLQWWEFDITWITIRALQGIGLAQKVRLVEAPPKQ

SEQ ID NO: 60 - protein, 272 aa, Anabaena variabilis
MTIATSTKPQINWVNTLFFLGLHIGALFAFVPGNFSWAAVGVALLLYWITGGLGITLGFHRLVTHRSFQTPKWLEYFLVLCGTLACQGGPIEWVGTHRIHHLHSDTDPDPHDSNKGFWWSHIGWLIYHSPSHADVPRFTKDIAEDPVYQFLQKYFIFIQIALGLLLLYLGGWSFVVWGIFFRIVWVYHCTWLVNSATHKFGYRTYDAGDKSTNCWWVAVLVFGEGWHNNHHAFQYSARHGLEWWEVDLTWMTVQLLQVLGLATNVKLADKKQ

SEQ ID NO: 61 - protein, 272 aa, Nostoc sp.
MTIATSTKPQINWVNTLFFLGLHIGALFAFIPSNFSWAAVGVALLLYWITGGLGITLGFHRLVTHRSFQTPKWLEYFLVLCGTLACQGGPIEWVGTHRIHHLHSDTDPDPHDSNKGFWWSHIGWLIYHSPSHADVPRFTKDIAEDPVYQFLQKYFIFIQIALGLLLLYLGGWSFVVWGVFFRIVWVYHCTWLVNSATHKFGYRTYDAGDRSTNCWWVAVLVFGEGWHNNHHAFQYSARHGLEWWEVDLTWMTVQLLQILGLATNVKLADKKQ

SEQ ID NO: 62 - protein, 272 aa, Anabaena variabilis; identical to SEQ ID NO: 61

SEQ ID NO: 63 - protein, 277 aa, Microcystis aeruginosa
MTVATSEKLPIDWVTLIYMAFIHLVALLAFLPGNFSWGAVGVTLILYWITGGLGITLGFHRLVSHRSFKTPKWLEYFLVFCGTLACQGGAIAWIGLHRIHHKYSDTAPDPHDSNKGFWWSHMGWMLHEIPADEEIARFTQDIADDPFYKFCQNYFVPIQIVLGLILYAIGGWPFVIWGIFVRLVLVFHSTWFVNSATHKFGYVSHESHDNSKNCWWVALLTFGEGWHNNHHAYQYSARHGLQWWEVDLTWMTIRLLQILGLATNIKLPPSTATATES

SEQ ID NO: 64 - protein, 273 aa, Moorea producens
MTIATSDNLRLDWVVITYMAFIHLAALFALLPSNFNWTAVGLMIFFHWVTGGLGVTLGWHRLVTHRSFQTPKWLEYFLVLCGTISCQGGVIDWVGLHRIHHLHSDTQPDPHDSHQGFWWSHMAWMLHKIPADEQISKFTKDISGDRVYQFLDKYFVLVQVVLGLLLYAMGGWPFVVWGIFVRLVLVFHCTWFVNSATHKFGYRTYESNDCSKNCWWVALLTYGEGWHNNHHAYQYSARHGIKWWEIDLTWMTIQFLQQLGLAKNVKLISPEQT

SEQ ID NO: 65 - protein, 277 aa, Microcystis aeruginosa
MTVATSEKLPIDWVTLIYMAFIHLVALLAFLPSNFSWGAVGVTLILYWITGGLGITLGFHRLVSHRSFKTPKWLEYFLVLCGTLACQGGAIAWIGLHRLHHKYSDTAPDPHDSNKGFWWSHMGWMLHEIPADEEIARFTQDIADDPFYKFCQNYFVPIQIVLGLILYAMGGWPFVIWGIFVRLVLVFHSTWFVNSATHKFGYVSHESHDNSKNCWWVALLTFGEGWHNNHHAYQYSARHGLQWWEVDLTWMTIRLLQILGLATNIKLPPTTAVTTES

SEQ ID NO: 66 - protein, 271 aa, Pleurocapsa sp.
MTVATSDKLFPAWSIIIYMATIHLVALLALFPSNFSWGALGISFVLYWLTGGIGITLGFHRLVSHRSFEVPKWLEYILVFCGTLACQGGPLYWIGGHRIHHKYSDTELDPHDSNKGFWWSHMGWMLCQNPARTEIPRYTKDIQDDPFYQFCEKYFIPIQIVLGLLLYWWGGWSFVIWGIFVRLVVVFHFTWFVNSATHKFGYQSYESNDNSRNCWWVALLTFGEGWHNNHHAYQYSARHGLEWWEIDLTWMTIRVLQILGLAKNVKLAPKQ

SEQ ID NO: 67 - protein, 277 aa, Microcystis aeruginosa
MTVATSEKLPIDWVTLIYMAFIHLVALLAFLPGNFSWGAVGVTLILYWITGGLGITLGFHRLVSHRSFKTPKWLEYFLVLCGTLACQGGAIAWIGLHRLHHKYSDTAPDPHDSNKGFWWSHMGWMLHEIPADEEIARFTQDIADDPFYKFCQNYFVPIQIVLGLILYAMGGWPFVIWGIFVRLVLVFHSTWFVNSATHKFGYVSHESHDNSKNCWWVALLTFGEGWHNNHHAYQYSARHGLQWWEVDLTWMTIRLLQILGLATNIKLPPTTATATES

SEQ ID NO: 68 - protein, 273 aa, Nostoc sp.
MTIATSTKPQINWVNTLFFIGLHIGALFAFVPGNFSWTAVGVGFLLYWVTGGLGVTLGFHRLVTHRSFQTPKWLEYLLVFFGTLSCQGGPIEWIGTHRIHHLHSDTDADPHDSTKGFWWSHMDWLIHYCPAHADVPRFTKDIAEDPVYQFLEKYFILIQVALGVLLLLLGGWPFVIWGIFVRIVWVYHCTWLVNSATHKFGYRSYDSGDQSTNCWWVAVLVFGEGWHNNHHAFQYSARHGLEWWEIDLTWMTVQLLQAVGLATNVKLAPTKSS

SEQ ID NO: 69 - protein, 277 aa, Microcystis aeruginosa
MTVATSEKLPIDWVTLIYMAFIHLVALLAFLPGNFSWGAVGVTLILYWITGGLGITLGFHRLVSHRSFKTPKWLEYFLVLCGTLACQGGAIAWIGLHRIHHKYSDTAPDPHDSNKGFWWSHMGWMLHEIPADEEIARFTQDIADDPFYKFCQNYFVPIQIVLGLILYAMGGWPFVIWGIFVRLVLVFHSTWFVNSATHKFGYVSHESHDNSKNCWWVALLTFGEGWHNNHHAYQYSARHGLQWWEVDLTWMTIRLLQILGLATNIKLPPTTATATES

SEQ ID NO: 70 - protein, 277 aa, Microcystis aeruginosa; identical to SEQ ID NO: 69

SEQ ID NO: 71 - protein, 277 aa, Microcystis aeruginosa; identical to SEQ ID NO: 69

SEQ ID NO: 72 - protein, 277 aa, Microcystis aeruginosa; identical to SEQ ID NO: 69

SEQ ID NO: 73 - protein, 277 aa, Microcystis aeruginosa
MTVATSEKLPIDWVTLIYMAFIHLVALLAFLPGNFSWGAVGVTLILYWITGGLGITLGFHRLVSHRSFKTPKWLEYFLVLCGTLACQGGAIAWIGLHRLHHKYSDTAPDPHDSNKGFWWSHMGWMLHEIPADEEIARFTQDIADDPFYKFCQNYFVPIQIVLGLILYAMGGWPFVIWGIFVRLVLVFHSTWFVNSATHKFGYVSHESHDNSKNCWWVALLTFGEGWHNNHHAYQYSARHGLQWWEVDLTWMTIRLLQILGLATNIKLPPSTATATES

SEQ ID NO: 74 - protein, 277 aa, Microcystis aeruginosa
MTVATSEKLPIDWVTLIYMAFIHLVALLAFLPGNFSWGAVGVTLILYWITGGLGITLGFHRLVSHRSFKTPKWLEYFLVLCGTLACQGGAIAWIGLHRLHHKYSDTAPDPHDSNKGFWWSHMGWMLHEIPADEEIARFTQDIADDPFYKFCQNYFVPIQIVLGLILYAMGGWPFVIWGIFVRLVLVFHSTWFVNSATHKFGYVSHESHDNSKNCWWVALLTFGEGWHNNHHAYQYSARHGLQWWEVDLTWMTIRLLQILGLATNIKLPPTTAVTTES

SEQ ID NO: 75 - protein, 277 aa, Microcystis aeruginosa; identical to SEQ ID NO: 74

SEQ ID NO: 76 - protein, 270 aa, Crinalium epipsammum
MTVATSTKLRTDWSIIIYMAAIHLAALPALLPSNFSWAAVGVAVLLHWITGGLGITLGYHRLVTHRSFQTPKWLEYFFIFCGTLSCQGGPIDWIGLHRIHHLHSDHNGDPHDSSQGFWWSHMGWMLHEIPSKAEIDRFTKDIADDPVYQFFQVAFIPMQVVLGLLLYMLGGWPFVVWGVFVRLVVVFHCTWFVNSATHKFGYSTYESNDNSKNCWWVALVTYGEGWHNNHHAYQYSARHGLQWWEIDLTWMTIQFLQLLGLATKVKVAEK

SEQ ID NO: 77 - protein, 277 aa, Microcystis aeruginosa
MTVATSEKLPIDWVTLIYMAFIHLVALLAFLPGNFSWGAVGVTLILYWITGGLGITLGFHRLVSHRSFKTPKWLEYFLVLCGTLACQGGAIAWIGLHRIHHKYSDTAPDPHDSNKGFWWSHMGWMLHEIPADEEIARFTQDIADDPFYKFCQNYFVPIQIVLGLILYAMGGWPFVIWGIFVRLVLVFHSTWFVNSATHKFGYVSHESHDNSKNCWWVALLTFGEGWHNNHHAYQYSARHGLQWWEVDLTWMTIRLLQILGLATNIKLPPTTAVTTES

SEQ ID NO: 78 - protein, 273 aa, Cyanobacterium aponinum
MTATTTTEKLAPAWSVIIYMTSIHLIALLAFLPSNFSWGAVGITAFLYWLTACIGVTLGYHRLVSHRSFETPKWLEYFLVFCGTLACEGGPISWVGLHRIHHKYSDHEGDPHDSNKGFWWSHMGWMFVKNPANNDVPKYTKDIQNDPFYVFCEKYFIPIQVVLGLLLFWWGGWSFVVWGIFLRLVLVFHVTWFVNSATHKFGYKSHESNDHSRNCWWVALLTFGEGWHNNHHAFQYSARHGLQWWEIDLTWMTIKLLAFFGLAKNIKLAPVKN

SEQ ID NO: 79 - protein, 277 aa, Microcystis sp.
MTVATSEKLPIDWVTLIYMAFIHLVALLAFLPGNFSWGAVGVTLILYWITGGLGITLGFHRLVSHRSFKTPKWLEYFLVLCGTLACQGGAIAWIGLHRLHHKYSDTAPDPHDSNKGFWWSHMGWMLHEIPADEEIARFTQDIADDPFYKFCQNYFVPIQIVLGLILYAMGGWPFVIWGIFVRLVLVFHSTWFVNSATHKFGYVSHESHDNSKNCWWVALLTFGEGWHNNHHAYQYSARHGLKWWEVDLTWMTIRLLQILGLATNIKLPPTTATATES

SEQ ID NO: 80 - protein, 274 aa, Crocosphaera watsonii
MTVAASKKLSYDWTIIIYMASIHLVALLAFLPGNFSWQAVGVTLLLYWVTGALGITLGFHRLVSHRSFKSPKWLEYLLVFFGTLACQGGPIQWVGLHRIHHKYSDTEPDPHDSNKGFWWSHMAWMLHQIPADNDVPRYTKDINEDPFYNFCQSFMIPIQIVLGLALYFLGGWSFVIWGIFVRLVMVFHFTWFVNSATHKFGYQSHESNDNSRNCWWVALLTFGEGWHNNHHAYQYSARHGLAWWEIDITWMTIQVLQFLGLAKNVKLAPVADKK

SEQ ID NO: 81 - protein, 274 aa, Crocosphaera watsonii; identical to SEQ ID NO: 80

SEQ ID NO: 82 - protein, 270 aa, Arthrospira platensis
MTIATETKLKLDWMTIVFMAFLHIGALFVLVPSNFSWAAVGLALFLHWVTGGLGITLGFHRLITHRSFETPKWLEYFLAFCGTLACQGGPIQWVGLHRMHHLYSDQTLDPHDSNKGFWWSHMGWMLREIPADADVPRFTKDINEDPVYLFLQNYFIPIQVALGVVLYLLGGWPLVVWGIFFRIVVVFHCTWFVNSATHKFGYQTYQSNDNSKNCWWVALVTYGEGWHNNHHAFQYSARHGLKWWEIDLTWMTIQLLQFLGLAKKVKLVEN

SEQ ID NO: 83 - protein, 270 aa, Spirulina platensis; identical to SEQ ID NO: 82

SEQ ID NO: 84 - protein, 270 aa, Arthrospira platensis; identical to SEQ ID NO: 82

SEQ ID NO: 85 - protein, 270 aa, Arthrospira sp.; identical to SEQ ID NO: 82

SEQ ID NO: 86 - protein, 270 aa, Arthrospira maxima; identical to SEQ ID NO: 82

SEQ ID NO: 87 - protein, 270 aa, Arthrospira platensis
MTIATETKLKLDWMTIIFMAFLHIGALFVLVPSNFSWAAIGVAVFLHWVTGGLGITLGFHRLITHRSFETPKWLEYFLAFCGALACQGGPIQWVGLHRMHHLYSDQTLDPHDSNKGFWWSHMGWMLREIPADADVPRFTKDINEDPVYLFLQNYFIPIQVALGVVLYLLGGWPLVVWGIFFRIVVVFHCTWFVNSATHKFGYQTYQSNDNSKNCWWVALVTYGEGWHNNHHAFQYSARHGLKWWEIDLTWMTIQLLQFLGLAKKVKLVEN

SEQ ID NO: 88 - protein, 270 aa, Arthrospira platensis; identical to SEQ ID NO: 87

SEQ ID NO: 89 - protein, 274 aa, Synechocystis sp.
MTLATYVKPPISWPVVGFMVLLHLSLLLVFIPGMFSWTGVGLALILHWVTAGLGITLGWHRLLTHRSFQVPKWLEYVLVFLGTLSMQGGPIWWVGLHRHHHLYSDQAVDHHDSCKGFWWSHFEWMLRDVPAEAEIPKFTKDIADDPFYRFLDENFVMIQVAFAILLYFVGGWQFVVWGVLVRLVTVYHTTWLVNSATHKFGYRTFDSADRSTNCWWVALLTFGEGWHNNHHAYQYSARHGLRWWEIDLTWMTIQVLEFLGLAKKVRLVEVPMTE

SEQ ID NO: 90 - protein, 272 aa, Nostoc sp.
MTIATSTKPQINWVNTLFFVGLHIGALFALVPSNFSWTAVGVGFLLYWITGGLGVTLGFHRLVTHRSFQAPKWLEYLLVLFGTLSCQGGPIEWVGTHRIHHLHSDTDPDPHDSNKGFWWSHMGWLIHKCPAHADVPRFTKDIAEDPVYQFLEKYFILIQVALGLLLLFLGGWPFVIWGIFVRIVWVYHCTWLVNSATHKFGYRSYESGDNSTNCWWVAVLVFGEGWHNNHHAFQYSARHGLEWWEIDLTWMTIQLLQLFGLATNVKLADKKQ

SEQ ID NO: 91 - protein, 270 aa, Halothece sp.
MTVATEKKLPFDWLTFLYITGIHLVALLAFFPNFFSWSAVGVALVMHWVTAGLGVTLGFHRLLSHRSLEVPKWLEYFLVFCGSLSAQGAVIDWVGLHRIHHKYSDRDTDPHDSNKGFWWSHIGWMLHEIPADQEIYKYTQDINSDPFYQFLSKYFILIQVAFGLLLYAIGGWSFVIWGVFVRLAVVFHCTWFVNSATHKFGYKTYESNDESRNCWWVALLTYGEGWHNNHHAYQYSARHGLQWWEIDITWMMIRVLQILGLAKNVKLIRE

SEQ ID NO: 92 - protein, 275 aa, Cyanobacterium stanieri
MTVSTSEKLPLAWNVIIYMATIHLVALFAFLPSNFSWGAVGLFFVLYWLTACIGITLGFHRLVSHRSFETPKWLEYLLVLCGSLACQGGPIQWVGLHRVHHKFSDHDGDPHDSNKGFWWSHMGWMFVQNPANQLVPKYTKDIQNDRFYQFCDKYFIPIQVALGLLLFWWGGWSFVVWGIFLRLVVVFHVTWFVNSATHKFGYVSHDSNDHSRNCWWVAVLTFGEGWHNNHHAYQYSARHGLQWWEVDLTWITIKTLSLFGLAKNIKLAPTTVPTK

SEQ ID NO: 93 - protein, 273 aa, Nostoc punctiforme
MTIATSTKPQINWVNTLFFIGLHIGALFAFVPGNFSWTAVGVGFLLYWVTGGLGVTLGFHRLVTHRSFQTPKWLEYLLVFFGTLSCQGGPIEWIGTHRIHHLNSDTDTDPHDSNKGFWWSHMGWLTHYCPAHADVPRFTKDIAEDPVYQFLEKYFILIQVALGVLLLLLGGWPFVIWGIFVRIVWVYHCTWLVNSATHKFGYQSYDSGDKSTNCWWVAVLVFGEGWHNNHHAFQYSARHGLEWWEIDLTWMTVQLLQAVGLATNVKLAPAKSS

SEQ ID NO: 94 - protein, 272 aa, Cyanothece sp.
MTVSTSAKLPLDWSVIIYMATIHLVALLAFLPSNFNWGAVTVAFILYVVTAGFGITLGFHRLVTHRSFQTPKWLEYILVFCGTIACQGGPIHWVGLHRMHHKYSDTELDPHDSNKGFWWSHLGWMLHNIPADQEIPRYTQDISDDPFYQFCQKNLIPIQVALGLLLFALGGWPFVIWGIFVRLVVVFHCTWFVNSATHKFGYQSHESKDLSKNCWWVALVTFGEGWHNNHHAYQYSARHGLEWWEIDLTWMFIRTLQILGLATNIKLAPKNN

SEQ ID NO: 95 - protein, 271 aa, Anabaena sp. 90
MTIATSKPPINWVNTLFFVSLHVGALFALIPSNFSWKAIGVALLLYWVTGGLGITLGYHRLVTHRSFQTPKWLEYVLVIFGTLSCEGGPIEWVGTHRIHHLHSDTESDPHDSNQGFWWSHMGWMIHFAPAHSQVPRFTKDIIDDPVYQFLQKNFILLQVALGIILYFLGGWPMVVWGIFARIIWVYHCTWLVNSATHKFGYRTYDSGDQSTNCWWVAILVFGEGWHNNHHAFQYSARHGLEWWEIDLTWMTIQLLQLLGLATNVKLAEKKA

SEQ ID NO: 96 - protein, 271 aa, Anabaena cylindrica
MTIATSKPQINWVNTLFFVGLHVGALFALVPSNFSWNAVGIALFLYWVTGGLGITLGYHRLVTHRSFQTPKWLEYVLVIFGTFSCEGGPIEWVGTHRIHHLHSDTEKDPHDSNKGFWWSHMGWMIHFAPAHDEVPRFTKDIIDDPVYQFLQKNFIFLQIALGLALFFLGGWSFVVWGIFFRIVWVYHCTWLVNSATHQFGYRSHESGDKSTNCWWVALLVFGEGWHNNHHAFQYSARHGLEWWEIDMTWMTIQLLQLLGLATNVKLADRKQ

SEQ ID NO: 97 - protein, 279 aa, Microcoleus sp.
MTIATSNPEKAEISSPLRPDWVTIIFMAVLHIGALFALLPSNFSWKAIGLAILLHWVTGGLGITLGFHRLVTHRSFQTPKWLEYFFVFCGTLACQGGPIRWVGLHRIHHLHSDTQPDPHDSNRGFWWSHMGWMLYNIPSDVEIPRYTKDISDDPVYQFLQNYFVPIQIVLGLIFYGLGGWPFVVWGIFARIVIVFHCTWLVNSATHMFGYQTYDANDHSRNCWWVALLTYGEGWHNNHHAYQYSARHGMKWWEIDLTWMTIQFLQLLGLAQKVKLVGKE

SEQ ID NO: 98 - protein, 273 aa, Cylindrospermopsis raciborskii
MTIATSTKHQINWVNTLFFIFLHIGALFAFIPGNFSWTAVGVALLLYWVTGGLGITLGFHRLITHRSFQSPKWLEYFLALCGTLACQGGPIEWVGTHRIHHLHSDTQEDPHDSNKGFWWSHIGWLIFHSPAHAQIPRFTKDIAEDRVYQFLQKYFIPIQLFLGLFLLMLGGWSFVVWGIFARIVWVYHCTWLVNSATHKFGYRTYPESGDRSTNCWWVAVLVFGEGWHNNHHAFQYSARHGLEWWEIDLTWMTIQLLQVLGLATNIKLAPRKA

SEQ ID NO: 99 - protein, 272 aa, Cyanothece sp.
MTVATSEKLSPAWHIIIYMATIHLVALLAFLPSNFNWGAVAVAFILYVVTAGIGVTLGFHRLATHRSFETPKWLEYILVFCGTLACQGGPIDWVGLHRMHHKYSDTELDPHDSNKGFWWSHLGWMLYNLPADKEIPRYTKDIADDPFYQFCQKNLIAIQVGLGLLLFALGGWPFVIWGIFVRLVVVFHCTWFVNSATHKFGYQSHESGDLSKNCWWVALVTFGEGWHNNHHAYQYSARHGLEWWEIDLTWMFIRTLQFLGLATNIKLAPTKE

SEQ ID NO: 100 - protein, 289 aa, Oscillatoria acuminata
MTIVSSTQETQETIGQDLSQMRPDWPVIIFMIAIHALALFAFLPSTFSWAAVGLAVLFHWVTGGLGVTLGFHRLVTHRSFTAPKWLEYFLVLCGTLSCQGSPIDWVGLHRAHHLHSDNSADPHDSNQGFWWSHMGWLFYHSPAEAEIPRLTKDIADDPFYQFCDKFLLPIQIALGISFYFLGEAISPGLGWSFVVWGIFVRLVVVFHCTWFVNSATHKFGYRTYDSEDRSTNCWWVALVTYGEGWHNNHHAYQYSARHGMQWWEIDFTWMTIQILQALGLAKNVKLVGK

SEQ ID NO: 101 - protein, 275 aa, Pleurocapsa sp.
MTVATSDKLSPAWSIIIYIATVHLIALLALFPSSFSWGAVGVAFVLYWLTACMGITLGFHRLVSHRSFEVPKWLEYVLVFCGTLACQGGPLYWIGGHRIHHKYSDTELDPHDSNKGFWWSHLGWMLWENASVKEDIPRYTKDIQDDPFYQFCEKYFIPIQIVLGLLLYWWGGWSFVIWGIFVRLVVVFHFTWFVNSATHKFGYQSYESNDNSRNCWWVALLTFGEGWHNNHHAYQYSARHGLEWWEIDLTWMTIRVLQILGLAKNVKLAPTRSSD

SEQ ID NO: 102 - protein, 280 aa, Acaryochloris marina
MTIATPPSQLKRDWTFIVVLIAIHIGALFAFVPSNFSWAAVGFAVFFHWVTGGLGITLGWHRMLTHRSFKTPKWLEYFLVFCGSLACEGGVIWWVGLHRNHHMHSDTDLDQHNSRNGFWWSHMGWMIHEIPAEADIDKLTKDINTDPFYRFLDDYFFPMQIVFGVLLYFIGGWPLVVWGIFVRLVLVYHCTWFVNSATHKFGYKTYESDDQSTNCWWVALLTYGEGWHNNHHAFPQSARHGLQWWEIDTTWMMVQLLQSLGLADKVRLPNAQQLASNDGT

SEQ ID NO: 103 - protein, 272 aa, Lyngbya sp.
MTIATSQKLNPEWKVIIFMVILHGGALFALLPSNFNWTAIGIAFLLHWVTGGLGITLGFHRLLTHRSFETPKWLEYFFVFCGTLACEGGVIDWVGSHRMHHLHSDHELDPHDSNKGFWWSHMGWMLYARPPKAEIDRLTQDIGADPVYLFFQNYFILIQVALGILLYLLGGWPFVVWGVFVRIIAVFHCTWLVNSATHKFGYRTFDCDDASTNCWWVAVLTYGEGWHNNHHAYQYSARHGLQWWEIDQTWMTIQFLQTLGLAKKVKLASKSS

SEQ ID NO: 104 - protein, 274 aa, Cyanothece sp.
MTVATSQKLPYDWTIIIYMASIHLVALLAFLPGNFSWQAVGVAFFLYWVTGALGITLGFHRLVSHHSFQAPKWLEYILVFFGTLACQGGPIQWVGLHRIHHKYSDTEPDPHDSNRGFWWSHIGWMLHHIPADNDVPRYTQDINEDPFYKFCQTFMIPIQIVLGLILYFLGGWSFVIWGIFVRLVMVFHFTWFVNSATHKFGYQSHESNDNSRNCWWVALLTFGEGWHNNHHAYQYSARHGLAWWEIDITWMTIQVLQFLGLAKNVKLAPVSDNK

SEQ ID NO: 105 - protein, 274 aa, Cyanothece sp.; identical to SEQ ID NO: 104

SEQ ID NO: 106 - protein, 274 aa, Cyanothece sp.
MTVATSQKLPLDWAIILYMATIHLVALLAFLPSNFSWGAVGVTLFLYWVTGALGITLGFHRLVTHRSFQAPKWLEYFLVFCGTLACQGGPIQWVGLHRIHHKYSDTQNDPHDSNLGFWWSHLGWMLHKIPADNDVPRYTQDIAEDPFYQFCQKYMILIQVALGLLLYFLGGWSFVIWGIFVRLVMVFHFTWFVNSATHKFGYQSHESDDHSKNCWWVAVLTFGEGWHNNHHAYQYSARHGLAWWEIDITWMTIQLLQFLGLATNVKLAPTEKTN

SEQ ID NO: 107 - protein, 273 aa, Raphidiopsis brookii
MTIATSTKPQINWVNTLFFIFLHIGALFAFIPSNFSWTAVGVALLLYWVTGGLGVTLGFHRLITHRSFQSPKWLEYFLALCGTLACQGGPIEWVGTHRIHHLHSDTPEDPHDSNKGFWWSHIGWLIFHSPAHAQIPRFTKDIVEDRVYQFLQKYFIPIQFFLGLFLLMLGGWSFVVWGIFARIVWVYHCTWLVNSATHKFGYRTYPESGDRSTNCWWVAVLVFGEGWHNNHHAFQYSARHGLEWWEVDLTWMTIQLLQFLGLATNIKLAPKKA

SEQ ID NO: 108 - protein, 274 aa, Gloeocapsa sp.
MTIATSTKPPLNWAFILFIAFVHIGAAFALLPSNFSWSAVGLALLLHWITGGLGITLGFHRLVTHRSFQTPKWLEYFFVFCGTLACQGGVLDWVGLHRLHHLYSDQTPDPHDSNQGFWWSHIGWMLFHTPIQSEVPRYIKDIADDPVYKFFDKYFIPIQFALGLLLYFIGGWSFVVWGIFVRLVLVWHCTWFVNSASHIFGYSTYKSNDNSKNCWWVALVTYGEGWHNNHHAFQYSARHGLQWWEIDLTWMTILFLQTLGLATKVKLAPKESLN

SEQ ID NO: 109 - protein, 274 aa, Cyanothece sp.
MTVATSQKLPLDWAIILYMATIHLVALLAFLPSNFSWGAVGVTLFLYWVTGALGITLGFHRLVTHRSFQAPKWLEYFLVFCGTLACQGGPIQWVGLHRIHHKYSDTQNDPHNSNLGFWWSHLGWMLHKIPADNDVPRYTQDIAEDPFYQFCQKYMILIQVALGLLLYVLGGWSFVIWGIFVRLVMVFHFTWFVNSATHKFGYQSHESDDHSKNCWWVAVLTFGEGWHNNHHAYQYSARHGLSWWEIDITWMTIQLLQFLGLATNVKLAPTEKTN

SEQ ID NO: 110 - protein, 274 aa, Cyanothece sp.
MTVATSQKLPYDWTIIIYMASIHLIALLAFLPSNFSWQALGVAFFLYWVTGALGITLGFHRLVSHRSFQAPKLLEYLLVFLGTLACQGGPIQWVGLHRIHHKYSDTEPDPHDSNRGFWWSHLGWMLHHIPADNDVPRYTQDIKDDPFYNFCQNFMVPIQLGLGLILYFLGGWPFVIWGIFVRLVMVFHFTWFVNSATHKFGYQSHESNDNSRNCWWVALLTFGEGWHNNHHAYQYSARHGLAWWEIDITWMTIKILEFVGLAKNVKLAPVSDNK

SEQ ID NO: 111 - protein, 277 aa, Stanieria cyanosphaera
MTVATSDRLPPDWLNISYFGLIHLVALLAVFPQNFSWGAVGVAFLLYWLTAGIGITLGFHRLVTHRSFETPKWVEYILVFCGTLACEGGPLDWVGAHRIHHKHSDTELDPHDSNRGFWWSHIGWMMCEHPVNQEIPRYTKDIADDPFYQFCQKYLVPIQVGLGLLLYFIGGWSYVIWGIFVRLVVVFHVTWLVNSATHKFGYKSHESNDNSTNCWWVALLTFGEGWHNNHHAYQYSARHGLRWWEIDATWMTIKLLEFLGLAKNIKLPPTQTKQTQV

SEQ ID NO: 112 - protein, 270 aa, Trichodesmium erythraeum
MTISTSKTLSLDWSIIGFMAFLHIGALFALFPSNFSWTAVGLALLLHWVTGGLGITLGFHRMLTHRSFKTPKWLEYFFIFCGTLACEGGVCDWVGLHRIHHQHSDNEQDPHDSHRGFWWSHMGWMLHKLPMKSEISRYTKDIANDPVYKFLQANFILIQFAFGILLFFIGGWSFVVWGIFVRIVAVFHCTWLVNSATHKFGYRTYDSGDSSTNCWWVAVLTYGEGWHNNHHAFQYSARHGLKWWEIDTTWMMIQLLQAFGLATNVKLATK

SEQ ID NO: 113 - protein, 284 aa, Synechococcus sp.
MTAAQELPPQNLKPAWKTIFFMALVHLGALCALIPGTFSWSGLAVGGVLFWLTGGLGITLGWHRLLTHRSFQLPKFLEYFFAFCGALACEGGVIEWVGLHRNHHLHSDHEQDQHNSRRGFWWSHMGWMLSEIPAKVEIDRLTKDINQDPVYRFLDDHFILIQVAFGLVLYALGGWSWVIWGIFVRLVAVYHVTWFVNSATHQFGYRTFETDDYSTNCWWVALLAFGEGWHNNHHTYPHSARHGLQWWEFDLTWQTIRLLKGLGLAKKVRLVTATGGMAGEPVED

SEQ ID NO: 114 - protein, 271 aa, Oscillatoria nigro-viridis
MTIATQEKLQYNWKHVISLAGLHIGALFALLPSNFNWTAVGLTVFMHWITGGLGITLGWHRLLTHRSFQTPKFVEYFLAFCGALACQGGIIDWVGLHRLHHTHSDESALDPHNAQKGFWWSHIGWMLYKGPAHAEISRCTKDISSDPFYQFLQNYFLPMQIVFGVLLYLAAGWPFVVWVIFVRLVLVYHCTWFVNSATHKFGYRTYETEDSSTNCWWVAVLTYGEGWHNNHHAFQYSARHGMEWWEIDFTWMTIQVLEALGLATKVKLPAK

SEQ ID NO: 115 - protein, 272 aa, Microcoleus vaginatus
MTIATQEKLQYNWKHVISLAGLHIGALFALLPSNFNWTAVGLTVFMHWITGGLGITLGWHRMLTHRSFQTPKFVEYFLAFCGALACQGGIIDWVGLHRLHHTHSDESALDPHNAQKGFWWSHIGWMLYKGPAHAEISRCTKDISSDPFYQFLQNYFLPVQIAFGVLLYLAAGWPFVVWVIFVRLVAVYHCTWFVNSATHKFGYRTYETEDSSTNCWWVAVLTYGEGWHNNHHAFQYSARHGMKWWEIDFTWMTIQVLEALGLATKVKLPAAK

SEQ ID NO: 116 - protein, 277 aa, Gloeocapsa sp.
MTIATSKQNPLPLDWGTIIYMFLIHGVALLALFPRFFNWQAVGLMLLFHWITAALGITLGFHRLVAHRSFQTPKLVEYFFIFCGTLACQGGPIDWIGLHRIHHKYSDTDLDPHDSNKGFWWSHLGWMFHQIPADKDILRYTKDINSDPFYQFCQKFFIPIQVVLGLVLFYFGGWSWVIWVIFVRLVVVFHCTWFVNSATHKFGYRTHESGDDSRNCWWVALLTYGEGWHNNHHAYQYSARHGLQWWEIDLTWMTIRLLGFLGLANNIKLPEPEASGS

SEQ ID NO: 117 - protein, 272 aa, Richelia intracellularis
MIITTSTKRSINWVNTLFFVIVHTGAIFAFFPQFFSWKAVGIGLLFYWITGGLGVTLGFHRLVTHRSFHSPKWLEYLLVLFGTISCQGGPIEWIGTHRIHHLYSDQEKDPHDSNQGFWWSHMGWLIYNRHDLSELPKFTKDISEDLVYQFLEKNMIQLQVLLGIGLLFWGGWSFVFWGVFARIVWVWHCTWLVNSATHKFGYTSHESSDKSTNCWWVAILVFGEGWHNNHHAYQYSARHGLEWWEVDLTWMTIQFLELLGLAKNIKLAKKYD

SEQ ID NO: 118 - protein, 271 aa, Oscillatoria sp.
MTIATEPKLELNWTHIISLAVVHIGALCALFPSNFNWAAVSLAIILHWVTGGLGITLGWHRLVSHRSFQTPKWVEYFLVFCGVLSGQGGVIHWVGLHRVHHQHSDQGALDPHDSRRGFWWSHIGWMFYHCPALDTIDRCTKDIADDPVYKFFQNYFLPIQIAFGLLLLLIGGLPFVVWAIFVRLALVYHCTWFVNSATHKFGYRTYETSDSSTNCWWVALVTYGEGWHNNHHAFQYSARHGLKWWEIDLTWMTIQLLQALGLATKVKLVQK

SEQ ID NO: 119 - protein, 318 aa, Synechocystis sp.
MLNPLNIEYLYLSKLFDNSLIVFNKRQLFRFFVRFFFMTAALPNDSKPKLTPAWTVIFFFTSIHLVALLAFLPQFFSWKAVGMAFLLYVITGGIGITLGFHRCISHRSFNVPKWLEYIFVICGTLACQGGVFEWVGLHRMHHKFSDTTPDPHDSNKGFWWSHIGWMMFEIPAKADIPRYTKDIQDDKFYQFCQNNLILIQVALGLILFALGGWPFVIWGIFVRLVFVFHFTWFVNSATHKFGYVSHESNDYSRNCWWVALLTFGEGWHNNHHAYQYSARHGLQWWEVDLTWMTIKFLSLLGLAKDIKLPPETAMANKA

SEQ ID NO: 120 - protein, 318 aa, Synechocystis sp.; identical to SEQ ID NO: 119

SEQ ID NO: 121 - protein, 318 aa, Synechocystis sp.; identical to SEQ ID NO: 119

SEQ ID NO: 122 - protein, 318 aa, Synechocystis sp.; identical to SEQ ID NO: 119

SEQ ID NO: 123 - protein, 318 aa, Synechocystis sp.; identical to SEQ ID NO: 119

SEQ ID NO: 124 - protein, 318 aa, Synechocystis sp.; identical to SEQ ID NO: 119

SEQ ID NO: 125 - protein, 318 aa, Synechocystis sp.; identical to SEQ ID NO: 119

SEQ ID NO: 126 - protein, 281 aa, Synechocystis sp.
MTAALPNDSKPKLTPAWTVIFFFTSIHLVALLAFLPQFFSWKAVGMAFLLYVITGGIGITLGFHRCISHRSFNVPKWLEYIFVICGTLACQGGVFEWVGLHRMHHKFSDTTPDPHDSNKGFWWSHIGWMMFEIPAKADIPRYTKDIQDDKFYQFCQNNLILIQVALGLILFALGGWPFVIWGIFVRLVFVFHFTWFVNSATHKFGYVSHESNDYSRNCWWVALLTFGEGWHNNHHAYQYSARHGLQWWEVDLTWMTIKFLSLLGLAKDIKLPPETAMANKA

SEQ ID NO: 127 - protein, 276 aa, Rivularia sp.
MTIATSRDKGPLNWLHITFFVGLHFGVLLAPFPAFFNWKAVGVALLLYWVTGGLGITLGFHRLITHRSFQTPKWLEYFLAFCGTLACQGGPIAWVGMHRIHHLHSDQPLDPHDSNKGFFWSHMGWMFHHSPAFDDVPRFTKDIKDDPFYIFLEKNMLLIQIALGGVLLALGGWSFVVWGVFVRLIFVWHCTWFVNSATHKFGYQTYDAKDHSTNCWWVALLTYGEGWHNNHHAFQYSARHGLQWWEIDLTWMTIQLLQFLGLATNVKLAPVKQSST

SEQ ID NO: 128 - protein, 276 aa, Calothrix sp.
MTTATTPAKPTRNWFHIGFFGPLHLGALLALFPAFFSWKAVGAALLLYWITGGLGITLGFHRLITHRSFQTPKWLEYFLATCGTLACQGGPIDWVGLHRIHHLHSDQEQDPHDSNRGFWWSHFGWMLHQSPAFAEVPRYTKDIYDDPYYKFLQKNMVLLQVALGAALLLFGGWSCVVWGIFVRLVFVWHCTWFVNSATHKFGYKTYDSKDHSTNCWWVALLTYGEGWHNNHHAFQYSARHGMKWWEIDLTWMTIQLLQLVGLATNVKLPDKKSITV

SEQ ID NO: 129 - protein, 274 aa, Xenococcus sp.
MTIATSERRPIAWGIVLYMASIHLIALLAFFPSNFSWGAVGVAFALYCITEWGITVGFHRLATHRSFEAPKFVEYFFIFLGTLACQGGPFEWIGLHRVHHKFSDTQPDPHDSNRGFWWSHMGWMLFENPADEEAKRYIKDIENDSFYQFCQKFMVPIQVVLGLILFFSGGWSFVIWGIFVRLVVVFHCTWLVNSATHKFGYQSHESNDSSRNCWWVAALTFGEGWHNNHHAYQYSARHGLAWWEIDITWMIIKTLETFGLAKNIKLPPKEAQQA

SEQ ID NO: 130 - protein, 271 aa, Fischerella sp.
MTIATSTKSRSHLVHITFLSVLHAAALLALFPAFFSWKAVGVCLFLHWVTGGLGITLGYHRLVTHRSFQTPKWLEYFLVLCGTLSCEGGPIEWVGMHRMHHLYSDKELDPHDSNKGFWWSHMGWMFFHSPLQPEVPRFTKDIGDDPVYQFFQNNLIWLQVALGVVLFLLGGWSFVIWGVFVRLVFVWHCTWLVNSATHKFGYRTYESGDRSTNCWWVALLTYGEGWHNNHHAFQYSARHGLQWWEIDMTWMTVKLLQALGLATNVKLADNK

SEQ ID NO: 131 - protein, 274 aa, Cyanobacteria
MIVTTSKKLPRDWTIIVYMILIHSFALFALSPSNFSWGAVGIAIFFYWVTGALGITLGFHRLVSHRSFQTPKWLEYLLVFCGTLACQGGVIQWVGLHRIHHKFSDTKSDPHDSKRGFWWSHMGWIIHKIPADKYISLYTQDISEDHFYKFCQIFMIPIQLFLGVFLYFLGGKAFVVWGIFVRLVVVLHFTWFVNSATHKFGYQSYKSNDNSRNCWWVALLTFGEGWHNNHHAYQYSARHGITWWEIDITWITIKFLRFLGLAKNVKLAPKSSNI

SEQ ID NO: 132 - protein, 277 aa, Rivularia sp.
MTTNLSPASLPQLNPPSPRLKVIIFTVILHLAVLLAFLPSNFSWAAVGVAVLLHWMTIGLGIAFGFHRLATHRSLQVPKWLEYFLILCGTLALQGGVFGWVGYHRVHHLHSDEQGDPHTPNEGFWWSHIGWLMHTVPARPELPRMTKDIADDQFYRFCHRYYIELQVALGVVLYLLGGWSFVVWGIFVRLFVGIHSTMFVNSVCHTIGYRNHDSGDRSTNCWWVALLTFGEGWHNNHHAFQYSARHGLQWWEIDMTWMTIRLLQFLGLATNVKLPQK

SEQ ID NO: 133 - protein, 321 aa, Prochlorococcus marinus
MYSLRSFDRSLMASRVIPTPSTISRPVASHKALSLSTKLQRPFTSKAIGPPKESRRWGTIGFMIAIHALSILALLPRFWSWQAITTLLVLYWVTACLGVTIGYHRLLSHRSFRVPRWLERFFATCGTLSCQHGPLDWVGLHRHHHKFSDTDPDHHNSHKGFWWSHMGWMFEPIPAMRTVPRLTVDLADDPYYRWLNKYFLLLQLPLAGLLFWIGSVSGAGGWTLVLWGIPLRLTIVYHITWLVNSASHCWGTVAFDSGDNSRNNPWVAALTFGEGWHNNHHAFPYSAIHGLQRGQIDLTWQHIRLMRALGLARKIRLPITS

SEQ ID NO: 134 - protein, 312 aa, Prochlorococcus marinus
MNSVIFQETAKLKKPVPAEKVIELSEKLLEPSSHSKRYPPRLHKTWGTIVFMVAIHILSLVAIQPKFWSLPAVTSLLFFYWVTACLGVTLGYHRLLSHRSFIVPRWLERFFATCGAISCQHGPIDWVGLHRHHHSFSDTEVDHHNSKKGFWWSHMGWMFKDVEALKTVPKLSADLIKDPYYRFLNKYFLFLQIPIGLSLYAIGQKLGVGGWALVLWGIPLRLVVVYHITWLVNSATHCWGKAPFESGDSSKNNAWVAALTFGEGWHNNHHAFPNSAKQGLFRGQIDITWEHIKILAKFGLAKKVKLPSRSYY

SEQ ID NO: 135 - protein, 312 aa, Prochlorococcus marinus
MNSVIFQETAKLKKPVPAEKVIELSEKLLEPSSHSKRYPPRLHKTWGTIVFMVTIHILSLIAIQPKFWSLPAVTSLLFFYWVTACLGVTLGYHRLLSHRSFVVPRWLERFFATCGAISCQHGPIDWVGLHRHHHSFSDTEVDHHNSKKGFWWSHMGWMFKDVEALKAVPKLSADLIKDPYYRFLNKYFLFLQIPIGLSLYAIGQKLGVGGWALVLWGIPLRLVVVYHVTWLVNSATHCWGKAPFESGDSSKNNAWVAALTFGEGWHNNHHAFPNSAKQGLFRGQIDITWEHIKILAKFGLAKKVKLPSRSYY

SEQ ID NO: 136 - protein, 328 aa, Prochlorococcus marinus
MIILHIGLFENSFSNIMKSVIFQETANLKKPVPAEKVIELSDKLLEPSSHSKRYPPRLHKTWGTIFFMIAIHLLSLLALQPQFWSMPAVTALFFFYWLTACLGVTLGYHRLLSHRSFVVPKWLERFFATCGAISCQHGPIDWVGLHRHHHSFSDTEVDHHNSKRGFWWSHMGWMFKDVEALKAVPKLSADLIKDPYYRFLNKYFLFLQIPIGLCLYAIGQKLGVGGWALVLWGIPLRLVVVYHITWLVNSATHCWGKAPFESGDGSKNNAWVAALTFGEGWHNNHHAFPNSARQGLFRGQIDLTWEHIKILAKLGFAKKVKLPSRSYY

SEQ ID NO: 137 - protein, 312 aa, Prochlorococcus marinus
MNSVIFQETAKLKKPVPAEKVIELSEKLVEPSRHSKKYPPRLHKTWGTIVFMVAIHILSLIALQPKFWSLPAVTSLLFFYWVTACLGVTLGYHRLLSHRSFIVPRWLERFFATCGAISCQHGPIDWVGLHRHHHSFSDTEVDHHNSKKGFWWSHMGWMFKDVEALKAVPKLSADLIKDPYYRFLNKYFLFLQIPIGLSLFAIGQKLGVGGWALVLWGIPLRLVVVYHITWLVNSATHCWGKAPFESGDSSKNNAWVAALTFGEGWHNNHHAFPNSAKQGLFRGQIDITWEHIKILAKFGLAKKVKLPSRSYY

SEQ ID NO: 138 - protein, 273 aa, Richelia intracellularis
MTRNLPQLNSVSPRPKVIISTCILHLAVLLVFLPHTFSWNSLCIAVFLHWVTIGLGISFGFHRLATHQSLQAPKWLEYFLILCGTLALQGGVLGWVGYHRAHHLYSDQEKDPHNSNQGFWWSHINWLMHSVPTRKEIPRLTKDIAKDPFYRFCHEYYIELQLFLAIILYLLGGWSFVIWGIFVRLFVGTHSTMFVNSACHMVGYRSYDSEDCSTNCWWVALLTFGEGWHNNHHAFQYSARHGLEWWEIDVTWITIRLLQSLGLVTNVKTIQRE

SEQ ID NO: 139 - protein, 314 aa, Prochlorococcus marinus
MVSSVIGPSAPLRRPVSASKALAASTKLQIQTPINNKNKLKPLGKFERRWGTIIFMFAIHALTIFALQPKFWSVQAVSALLVMYWVTACLGVTLGYHRLLSHRSFQVPRWLERFFATCGALSCQHGPIDWVGLHRHHHTFSDTDADHHNSNKGFWWSHMGWMFEDVPALGTVPRFTGDLVKDPYYLFLNQHFLLLQIPLGAFLFWIGTISEVGGWSMVLWGIPLRLVVVYHVTWLVNSATHCWGSVAYESGDRSRNNRWVAALTFGEGWHNNHHAFPSSAKQGLQKGQIDLTWEHIRFLKRLGLARKIRLPMTF

SEQ ID NO: 140 - protein, 312 aa, Prochlorococcus marinus
MNSVIFQETAKLKRPVPAEKVAELHYKFLAPSSHSKKYPPRLHKTWGTIVFMIAIHVLSLVALQPQFWSLPAVTALFFFYWLTACLGVTLGYHRLLSHRSFVVPRWLERFFATCGAISCQHGPIDWVGLHRHHHSFSDTEVDHHNSKRGFWWSHMGWMFKDVEALKAVPKLSADLIKDPYYRFLNKYFLFLQIPIGLTLYAIGQKLGVGGWAMVLWGIPLRLVVVYHITWLVNSATHCWGEAPFESGDESKNNAWVAALTFGEGWHNNHHAFPNSARQGLFKGQIDLTWEHIKILAKLRLAKKVKLPSRSYY

SEQ ID NO: 141 - protein, 312 aa, Prochlorococcus marinus; identical to SEQ ID NO: 140

SEQ ID NO: 142 - protein, 300 aa, Synechococcus sp.
MVSSQPADSRELRVRAAVTGPRGPLPARQRRLKSGTTGFMLVNHILATVALLPQFWSWQAIVAFGVLYWMTVLGVTLGLHRLVAHRSLVVPVWLERVLVIMGTLACQSGPIEWVGLHRHHHRFSDQPTDHHDAGRGLWWSHSEWMLHEIPALKELDRYAGDLQCDPFYRWLDRWFLLLQIPLGLGLYWYGEAAQVHGGGLGLVLWAIPLRLVVVYHVTWLVNSATHAFGYRNFDSPDLSRNCWWVAVLSFGEGWHNNHHAHPASARHGLRWFEFDITWQHVRLLKRFGLARRVREATYNP

SEQ ID NO: 143 - protein, 306 aa, Prochlorococcus marinus
MVSPLPVSSATNRQQRLWAAVKAHREPLPAKQRRFKWGTTSFMLAMHVAATFALLPRFWSWQGLVAFAVLYWTTVLGVTLGLHRLVAHRSFVVPVWLERILVIMATLACQSGPIEWVALHRHHHRYSDQTNDHHDAGRGLWWAHSEWMLHEIPALDHMDRFAGDLLADPFFRWLDRWFLLLQIPLGLGLYWYGEVAQVHGGGLGLVLWAIPLRLVVVYHVTWFVNSATHAFGYRNFDSPDLSRNCWWVAVLSFGEGWHNNHHAYPSSARHGLRWFEFDITWQHVKALRRLGLARRVQQARYVSGAS

SEQ ID NO: 144 - protein, 312 aa, Prochlorococcus marinus
MNSVIFQETAKLKKPVPAEKVIELSEKLLEPSSHSKRYPPRLHKTWGTIVFMVAIHILSLIALQPKFWSLPAVTSLLFFYWVTACLGVTLGYHRLLSHRSFIVPRWLERFFATCGAISCQHGPIDWVGLHRHHHSFSDTEVDHHNSKKGFWWSHMGWMFKDVEALKAVPKLSADLIKDPYYRFLNKYFLFLQIPIGLSLYVIGQKLGVGGWALVLWGIPLRLVVVYHVTWLVNSATHCWGKAPFESGDSSKNNAWVAALTFGEGWHNNHHAFPNSAKQGLFKGQIDITWEHIKILAKFGFAKKVKLPSRSYY

SEQ ID NO: 145 - protein, 328 aa, Prochlorococcus marinus
MIILHIGLFENSSSNIMKSVIFQETAKLKRPVPAEKVLELHYKFLAPSSHSKNYPPRLHKTWGTIFFMIAIHVLSFVALQPQFWSLPAVTALFFFYWLTACLGVTLGYHRLLSHRSFVVPKWLERFFATCGAISCQHGPIDWVGLHRHHHSFSDTEVDHHNSKRGFWWSHMGWMFKDVEALKAVPKLSADLIKDPYYRFLNKYFLFLQIPIGLCLFAIGQKLEVGGWAMVLWGIPLRLVVVYHITWLVNSATHCWGKAPFESGDESKNNAWVAALTFGEGWHNNHHAFPNSARQGLFKGQIDLTWEHIKLLAKLGYAKKVKLPSRTYY

SEQ ID NO: 146 - protein, 273 aa, Richelia intracellularis
MTRNLPQLNSVSPRPKVIISTCILHLAVLLVFLPHTFSWNSVCIAVFLHWITIGLGISFGFHRLATHQSLQAPKWLEYFLILCGTLALQGGVLGWVGYHRAHHLYSDQEKDPHNSNQGFWWSHINWLMHSVPTRKEIPRLTKDIAKDPFYRFCHEYYIELQLFLAIILYLLGGSSFVIWGIFVRLFVGTHSTMFVNSACHMVGYRSYDSEDCSTNCWWVALLTFGEGWHNNHHAFQYSARHGLEWWEIDVTWITIRLLQSLGLVTNVKTTQRE

SEQ ID NO: 147 - protein, 306 aa, Prochlorococcus marinus
MVSPLPVSSATNRQQRLWAAVKAHREPLPAKQRRLKWGTTSFMLAMHVAATFALLPRFWSWQGLVAFAVLYWTTVLGVTLGLHRLVAHRSFVVPVWLERVLVIMATLACQSGPIEWVALHRHHHRYSDQTNDHHDAGRGLWWAHSEWMLHEIPALDHMDRFAGDLLADPFFRWLDRWFLLLQIPLGLGLYWYGEVAQVHGGGLGLVLWAIPLRLVVVYHVTWFVNSATHAFGYRNFDSPDLSRNCWWVAVLSFGEGWHNNHHAYPSSARHGLRWFEFDITWQHVKALRRLGLARRVQQARYVSGAS

SEQ ID NO: 148 - protein, 310 aa, Synechococcus sp.
MTTSVITAPSPPSRPAASHEALKLAALLHQPRKGHMAVQPDQAKRWGTIGFMITIHILSIVALLPQFWSWQAVSALTILYWVTACLGVTIGYHRLLSHRSFRVPTWLERFFATCGALSCQHGPIDWVGLHRHHHKFSDTDADHHNSHRGFWWSHMGWMFQPIPAMQAVPRLSGDLVSDPYYRWLNTNFLLLQLPLGLLLFWIGSATGVGGWAMVLWGIPLRLVLVYHVTWLVNSATHCWGTVAYDSGDASRNNKWVAALTFGEGWHNNHHAFPHSARHGLQPGQIDLTWQHIRLMRALGLASQIRLPVAS

SEQ ID NO: 149 - protein, 310 aa, Cyanobium gracile
MRVPLQDTSHSRKKAQLLAAMERPRPPLPANQRKFKTGTTGFMLAIHVGAVFALLPRFWSFQGVIVLAVLYWATVLGVTLGLHRLVAHRGFEAPRWVERLLVLMGTLACQSGPIEWVGLHRHHHLFSDQPNDHHDAARGLWWAHSEWMLHRIPALTEIHRFTGDMEKDPFYRWLDRWFLLLQLPLGAALYWYGNWAGVHGGGLGLVLWGIPLRLVLVYHVTWLVNSATHAFGYRNFDCPDRSRNCWWVALLTFGEGWHNNHHAYPHSARHGLRWFEFDITWQHIRALRALGLARKVRIAPVFGRRADAAA

SEQ ID NO: 150 - protein, 303 aa, Synechococcus sp.
MVSSQTADTRELRQRAAVMSPRDRLPARQRKFKMGTTSFMLVMHVLATVALLPHFWSWQGVVAFGVLYWLTVLGVTLGLHRLVAHRSLVVPVWVERILVLMGTLACQSGPIEWVGLHRHHHRFSDQPTDHHDAGRGLWWAHSEWMLHDIPALKELDRYAGDLQCDPFYRWLDRWFLLLQIPLGLGLYWFGEAAQVHGGGLGLVLWAIPLRLVVVYHVTWLVNSATHAFGYRNFDCPDLSRNCWWVALLSFGEGWHNNHHAHPASARHGLRWFEFDLTWQHVRLLKRLGLASRIRTARYVPGAS

SEQ ID NO: 151 - protein, 308 aa, Prochlorococcus marinus
MVSSLIEASAPLKKPVAADKAMAASKKLQGHVPRRIQNKRARKRWGTVIFMLTIHALTIFTLQAQFWSWLAFSGFIFLYWVTACLGVTTGYHRLLSHRSFQVPKWLERFFATCGALSCQHGPIDWGLHRHHHTFSDTEADHHDSNKGFWWSHMGWMFEDVPAMKAVPRLSGDLIKDPYYRFLNKNFLLLQLPLGATLYLIGQSAGVGGWAMVLWGIPLRLVFVYHITWLVNSATHCWGTIAYESGDNSKNNKWVAALTFGEGWHNNHHAFPNSAKHGLQRKQIDLTWQHIRFLKAIGLAKKIRLPVAS

SEQ ID NO: 152 - protein, 286 aa, Synechococcus sp.
MMAPRDRLPARQRKFKMGTTSFMLVMHVLATVALLPRFWSWQGVVAFGVLYWMTVLGVTLGLHRLVAHRSLVVPVWVERILVLMGTLACQSGPIEWVGLHRHHHRFSDQPTDHHDAGRGLWWAHSEWMLHDIPALKELDRYAGDLQCDPFYRWLDRWFLLLQIPLGLGLYWFGEAAQVHGGGLGLVLWAIPLRLVVVYHVTWLVNSATHAFGYRNFDCPDLSRNCWWVALLSFGEGWHNNHHAHPASARHGLRWFEFDLTWQHVRLLKRLGLASRIRTARYVPGAS

SEQ ID NO: 153 - protein, 261 aa, Synechococcus sp.
MLVNHILATVALLPQFWSWQAIVAFGVLYWMTVLGVTLGLHRLVAHRSLVVPVWLERVLVIMGTLACQSGPIEWVGLHRHHHRFSDQPSDHHDAGRGLWWSHSEWMLHEIPALKELDRYAGDLQCDPFYRWLDRWFLLLQIPLGLGLYWYGEAAQVHGGGLGLVLWAIPLRLVVVYHVTWLVNSATHAFGYRNFDSPDLSRNCWWVAVLSFGEGWHNNHHAHPASARHGLRWFEFDITWQHVRLLKRFGLARRVREATYNP

SEQ ID NO: 154 - protein, 338 aa, Cyanobium gracile
MAVTPLSRPGTQPGAPSGDRRDAGTKARHTRRPAAINRGPDSLVRARALHEPRRGEPRGVSPKGTRWGTIGFMVVIHILAVVALLPRFWSLEAACALLVMYWVTACLGVTIGYHRLLSHRSFRVPQWLERFFATCGALSCQHGPIDWVGLHRHHHKFSDTEADHHDSNKGFWWSHMAWMFEEIPAMAAVPRLTGDLAADPYYRWLNHSFLLLQVPLGLLLFWIGTVTGDGGWALVLWGIPLRLVIVYQCTWLVNSATHMWGEAVHDSGDKSKNNPWVAALTFGEGWHNNHHAYPHSARHGFGPRQFDLTWQHIRLMRALGLATRVRLPRAAAPTRAMS

SEQ ID NO: 155 - protein, 317 aa, Synechococcus sp.
MPQIGSAGQALRGHAAEHQDAAVRASVSGRRQPLPPSQRKYKFGTTSFMLAIHVGAVFALLPRFWSWQAVVVLAALYWTTVLGVTLGLHRLLAHRSFVAPKWLERTLVLMGSLACQSGPIEWVGLHRHHHKYSDQPNDHHDAGRGLWWSHSDWMLHDIPAVRQMARFTGDLQRDPLYRWLDRWFLLLQVPLGAALWWYGERAGVPGGGLGLVLWGIPLRLAIVYHVTWLVNSATHAFGYRNFDSPDLSRNCWWVAVLSFGEGWHNNHHAHPASARHGLRWFEFDITWQHIKLLRALGLAKRIRVAHVGQPLPSATSS

SEQ ID NO: 156 - protein, 286 aa, Synechococcus sp.
MQAPRGPLPARQRKLKMGTTSFMVVMHVLATVALLPRFWSWQGLVAFGVLYWVTVLGVTLGLHRLVAHRSLVVPVWVERMLVIMGTLACQSGPIEWVGLHRHHHRFSDQPIDHHDAGRGLWWSHSEWMLHDIPALKELDRYAGDLQVDPFYRWLDRWFLLLQIPLGLGLYWIGEAAQVHGGGIGLVLWAIPLRLVIVYHVTWLVNSATHAFGYRNFDCPDLSRNCWWVALLSFGEGWHNNHHAHPASARHGLRWFEFDLTWQHVRLLKRLGLASRVRTARYVPGAS

SEQ ID NO: 157 - protein, 303 aa, Synechococcus sp.
MVTTNPVDSGSQRDLRMRAAVMAPRAPLPQRQRRLKGGTTGFMVVMHVLATVALLPRFWSWQGIATFAVLYWMTVLGVTLGLHRLVAHRSFEVPSWLEKTLVLMGTLACQSGPIEWIALHRHHHRFSDQPNDHHDAGRGLWWSHSEWMLHEIPALEHKERFAGDLLSSPFYVWLDRWFLLLQIPLGLALYWYGNAADVHGGGLGLVLWAIPLRLVVVYHVTWLVNSATHAFGYRNFNCPDLSRNCWWVALLSFGEGWHNNHHAHPASARHGLRWFELDITWMHIRLLQKLGLTRRVRQARYPG

SEQ ID NO: 158 - protein, 304 aa, Synechococcus sp.
MVVAPTTAPTAAPVADDLHERLYGRRPPLPASQRKLKVGTTLFMLAMHVGAVFALLPRFWSWQGLVAFGVLYWMTVLGVTLGLHRLMAHRSFEAPKWLERVLVVMGTLGCQGGPVEWVGLHRHHHKFSDQPNDHHDAGRGLWWSHSEWMLHSIPALKHLDRFAGDMRRDPFYNWLDRFFLLLQLPLGAALYWIGENTGVHGGGLGLVLWAIPLRLTVVYHVTWLVNSATHVWGYRNFDCPDRSTNCWWVAILSFGEGWHNNHHAFPHSARHGLRWFEFDITWQHIRALKSLGLAKRIRLAHYRP

SEQ ID NO: 159 - protein, 310 aa, Prochlorococcus marinus
MVSSVIEATAPLRRPVSADKALAASEKLQVKTKKELRYPRKFKRRWGTVGFMGAIHALTIFALLPRFWSLQNIAALLILYWVTACLGVTLGYHRLLSHRAFKVPHWLERFFATCGALSCQHGPIDWVGLHRHHHTFSDTDADHHNSKKGFWWSHMGWMFEPIPALQTVPNFSGDLIQDPYYRFLNKNFLLLQIPLGALLYWIGHGSESGGWSMVLWGIPFRLVVVYHVTWLVNSATHCWGNVAFDSGDGSKNNWWVAALTFGEGWHNNHHAYPSSARQGLFKGQIDLTWQHIRLLHALGLAKKIRLPMKP

SEQ ID NO: 160 - protein, 300 aa, Synechococcus sp.
MTVAIPAATISAPARSAVLSAPHPDKSTHCDGPGRRWGTVGFMAGIHILAVVALLPMFWSWQAVTSLAVLYWVTACLGVTIGYHRLLCHRSFRLPQWLERFFATCGALSCQHGPIDWVGLHRHHHKFSDTDADHHNSNRGFWWSHMGWMFEEIPAMGAVPRFSADLAKDPYYRWLNNNFLLLQLPLAGLLFLIGSLTGAGGWALVLWGIPLRLVLVYHVTWLVNSATHCWGEAVHESGDKSRNNPWVAALTFGEGWHNNHHAFPHSAKHGLEPGQIDLTWQHIRLMRALGLAKQVRLPAG

SEQ ID NO: 161 - protein, 325 aa, Cyanobium sp.
MTATAPTRARARLGSRGTRQPAFINGGADGLRRAAALHLPRSGEPQAAIKQGTRWGTIGFMVAIHALSLVALLPRFWSVPAVASFLILYWVTACLGVTVGYHRMLSHRAFRVPRWLERFFTTCGALSCQHGPIDWVGLHRHHHKFSDTEVDHHSSLRGFWWSHMGWMFHTIPAMAAVPRLTGDLARDPYYRWLNNWFLLLQLPLAGLLFWIGTVTGAGGWALVLWGIPLRLVVVYHCTWLVNSATHCWGSVSHDSGDQSRNNPWVAALTFGEGWHNNHHAFPHSARHGFGRQLDLTWQHIRLMRRLGLASQVRLPAAAALRSLRG

SEQ ID NO: 162 - protein, 321 aa, Synechococcus sp.
MASRFHAVPNQVRSVEISLVTTTPASTTSERELRMRAAVMAPRDPLPRKQRKLKGGTTSFMVAMHVLATVALLPRFWSWQGAVAFGVLYWATVLGVTLGLHRLVAHRSFEVPRWLERVLVVMGTLAAQSGPIDWVALHRHHHKFSDQPNDHHDAGRGLWWSHSEWMLHEIPALEHKERFGGDLLSDRFYVWLDRWFLLLQIPLGLGLYWYGNAAQVHGGGLGLVLWAIPLRLVVVYHVTWLVNSATHAFGYRNFDCPDLSRNCWWVAVLSFGEGWHNNHHAFPASARHGLRWFEFDITWMHIRLLQRLGLTRRVRQARYPA

SEQ ID NO: 163 - protein, 310 aa, Synechococcus sp.
MVSSTIRTPSPPSRPAASHLAVAQASLLRRPRKGEPATSPKNHQHSVTIGFMIVIHALAAVALLPGFWSWPAVTSLLVLYWVTACLGVTLGYHRLLTHRSFRLPLWLERFFATCGALSCQHGPIDWVGLHRHHHKFSDTDADHHNSHRGFWWSHMGWMFHPIEAMPAVPRLTGDLLSDPYYRWLNAHFLWLQLPLGLLLFWIGTATGAGGWALVMWGIPLRLVVVYHVTWLVNSATHCWGNVVYDSGDASRNNKWVAALTFGEGWHNNHHAFPHSARHGMQPGQIDLTWEHIRLLRALGLATKVRLPVAS

SEQ ID NO: 164 - protein, 259 aa, Prochlorococcus marinus
MLAIHALTIFTLQAQFWSWLAFSGFIFLYWVTACLGVTTGYHRLLSHRSFQVPKWLERFFATCGALSCQHGPIDWVGLHRHHHTFSDTEADHHDSNKGFWWSHMGWMFEDVPAMKAVPRLSGDLIKDPYYRFLNKNFLLLQLPLGATLYLIGQSAGVGGWAMVLWGIPLRLVFVYHITWLVNSATHCWGTIAYESGDNSKNNKWVAALTFGEGWHNNHHAFPNSAKHGLQRKQIDLTWQHIRFLKAIGLAKKIRLPAAS

SEQ ID NO: 165 - protein, 310 aa, Synechococcus sp.
MASSVITAPSPPSRPAASHRAQALATLLRQPRNGETPAAPAEGLHWVTIGFMIVIHSLALLALLPMFWSWQAAASLLVLYWVTACLGVTIGYHRLLSHRSFQLPRWLERFFATCGALSCQHGPIDWVGLHRHHHKFSDTDADHHNSHRGFWWSHMGWMLQPVTAMQAVPRMTGDLVQDPYYRWLNNNFLLLQLPLAGLLFWIGTATGAGGWALVLWGIPLRLVLVYHVTWLVNSATHCWGTVAYDSGDASRNNKWVAALTFGEGWHNNHHAYPHSARHGLQQGQIDLTWEHIRLMRALGLATKVRLPVAS

SEQ ID NO: 166 - protein, 259 aa, Prochlorococcus marinus
MIAIHALSILALLPRFWSWQAITTLLVLYWVTACLGVTIGYHRLLSHRSFRVPHWLERFFATCGTLSCQHGPIDWVGLHRHHHKFSDTDPDHHNSHKGFWWSHMGWMFEPIPAMRVVPRLTVDLAADPYYRWLNKYFLLLQLPLAGLLFWIGSVSGAGGLALVLWGIPLRLTIVYHITWLVNSASHCWGTVAFDSGDNSRNNPWVAALTFGEGWHNNHHAFPHSAIHGLQRGQIDLTWQHIRLMRALGLARKIRLPITS

SEQ ID NO: 167 - protein, 259 aa, Synechococcus sp.
MIVIHALAVVALLPGFWSWPAVTSLLVLYWVTACLGVTLGYHRLLTHRSFRLPTWLERFFATCGALSCQHGPIDWVGLHRHHHKFSDTDADHHNSHRGFWWSHMGWMFHPIEAMPAVPRLTGDLASDPYYRWLNAHFLWLQLPLGLLLFWLGTATGAGGWALVLWGIPLRLVVVYHVTWLVNSATHCWGNVVFDSGDASRNNKWVAALTFGEGWHNNHHAFPHSARHGMQPGQIDLTWEHIRLMRALGLATKVRLPVAS

SEQ ID NO: 168 - protein, 310 aa, Synechococcus sp.
MASSVITAPSPPSRPAAPDVALKKAAALHQPRKGEAPVSVDQGKRWGTIGFMIGIHVLAIVALLPGFWSWQAVTTLLVLYWVTACLGVTIGYHRLLSHRSFRVPHWLERFFATCGALSCQHGPIDWVGLHRHHHKFSDTDADHHNSHRGFWWSHMGWMFEPIPAMQAVPRMSGDLVSDPYYRWLNNNFLLLQLPLAGLLFWIGTVTGAGGWALVLWGIPLRLVLVYHITWLVNSATHCWGEVVYESGDASRNNKWVAALTFGEGWHNNHHAFPHSARHGLQPGQIDLTWQHIRLMRAIGLASKIRLPVAS

SEQ ID NO: 169 - protein, 264 aa, Synechococcus sp.
MTIGFMIVIHSLALLALLPMFWSWQAVTSLLVLYWVTACLGVTIGYHRLLSHRSFQLPHWLERFFATCGALSCQHGPIDWVGLHRHHHKFSDTDADHHNSHRGFWWSHMGWMFEPVPAMQAVPRMTGDLAQDPYYRWLNNNFLLLQLPLAALLFWIGTATGAGGWALVLWGIPLRLVLVYHVTWLVNSATHCWGTVAYDSGDASRNNKWVAALTFGEGWHNNHHAYPHSARHGLQPGQIDLTWEHIRLMRALGLATKIRLPVAS

SEQ ID NO: 170 - protein, 273 aa, Gloeobacter violaceus
MTAQSPATQATPISWVFVGFMAFIHLGALAALWQFSWSALALAIFLHWALGCLGITLGFHRLLSHRSFTVPKPLEYFLALLGTLCLQGGPVLWVAHHRLHHAHSDTEADPHDSNRGFWWSHWGWMFYMPEERLNFDHYSRYAKDLARDPVHRFLNRNMIPLQLAFAALLYLLGGWPFVLWGIFVRLVVVFHCTWLINSACHQWGYRSHQTDDHSTNLWWAAILAYGEGWHNNHHAMPRSARHGLAWWEIDVTWSVIWILSKLGLARNVQLPTS

SEQ ID NO: 171 - protein, 334 aa, Cyanobium sp.
MPTPITPANPRGLGPTIPPAQVLELEPPSRRHERELRHGLQASRPPLPPRQRRYKAGTTGFMLALHVGAMAALLPRFWSWQGLLALAVLYWVTVLGVTLGLHRLVAHRSFSAPLWVERVLVLMGALACQSGPIEWVGLHRHHHRHSDQPNDHHDAGRGLWWSHSDWMLHEIPALQHVESLTGDLQRDPFYRWLDRWFLLLQLPLGAALYWVGERAGVHGGGLGLVLWAIPLRLVLVYHVTWLVNSATHAWGYRNFDCPDRSRNCWWVAILSFGEGWHNNHHAFPHSARHGLRWFEFDITWQHIRLLWALGWARRVRLAPVTACTDRPVRRGWRR

SEQ ID NO: 172 - protein, 307 aa, Synechococcus sp.
MNRAPSPPSRPVTSHKALKRAGLLHVRRKGGRSAAPSQSPRRGTIVFMVVLHTSAMVALLPRFWSWQAVLTLGILYWATACLGVTIGYHRLLSHRSFRVPKWLERFFATCGALSCQQGPITWAGLHRHHHKFSDTDADHHNSHRGFWWSHMGWMFETIPAMQAIPRLSGDLSSDPYYRWLNRNFLYLQFPLAFLLFVIGSITGAGGWALVLWGIPLRLVLVYHVTWLVNSATHCWGTIAYDSGDASRNNKWVAALTFGEGWHNNHHAFPHSACHGLQKGQIDLTWEHIRLMRALGLAKNIRLPIKPA

SEQ ID NO: 173 - protein, 312 aa, Synechococcus sp.
MTATVRRPSPPSARSLSRAEALHRPRRGEPTPSAPKGTRWATLGFMAVVHALAVVALLPRFWSVPAVASMLLLYWITGCLGVTLGYHRLLAHRALRVPQWLERFFATCGALSCQHGPIDWAGLHRHHHKFSDTEADHHNSHKGFWWSHMGWMFEEIPAMAAVPRLTGDLQRDPYYRWLNTNFLLLQIPVGLLLFWIGTATGAGGWALVLWGIPLRLVVLYHCTWLVNSATHCWGSVSHESGDESRNNPWVAALTFGEGWHNNHHAFPHSARHGFGARQFDLTWQHIRLLKALGLATQVRLPSAAGKATQAVI

SEQ ID NO: 174 - protein, 307 aa, Synechococcus sp.
MVTTPLASAATDRELRMRAAVMAPRGPLPRRQRRIKWGTTSFMLILHVLATVALLPRFWSWQGVVALAVLYWMTVLGVTLGLHRLVAHRSLSVPRWLERTLVIMGTLACQSGPIEWVGLHRHHHRFSDQANDHHDAARGLWWSHSEWMLHAIPALEHIDRYAGDLQADPFYRWLDRWFLLLQIPLGLALYAYGQMADVHGGGLGLVLWAIPLRLVIVYHVTWLVNSATHACGYRNFDCPDLSRNCWWVALLSFGEGWHNNHHAHPASARHGLRWFELDITWLHIRLLRRLRLAHRIRQARYAPSGLP

SEQ ID NO: 175 - protein, 287 aa, Synechococcus sp.
MRAAVMAPRQTLPSQQRRLKGGTTSFMVVIHVLATVALLPRFWSWQGLVAFGVLYWMTVLGVTLGLHRLVAHRSFEVPGWLERVLVVMGTLACQSGPIDWVALHRHHHRFSDQPNDHHDAGRGLWWSHSEWMLHDIPALKEKHRYAGDLLSDRFYVWLDRWFLLLQIPLGLALYWYGEAAGVHGGGVGLVLWAIPLRLAVVYHVTWLVNSATHAFGYRNFNSPDLSRNCWWVAVLSFGEGWHNNHHAYPDSARHGLRWFEFDITWMHIRLLRKLGLTRKVRQARYSG

SEQ ID NO: 176 - protein, 303 aa, Synechococcus sp.
MVTTSPADTGSQRELRMRAAVMAPRAPLPRRQRRLKGGTTSFMVVMHVLATVALLPRFWSWQGLAAFAVLYWMTVLGVTLGLHRLVAHRSFEVPSWLEKILVVMGTLACQSGPIEWIALHRHHHRFSDQPNDHHDAGRGLWWSHSEWMLHEIPALEHKERYAGDLLSSPFYVWLDRWFLLLQIPLGLALYWYGNAAGVHGGGLGLVLWAIPLRLVVVYHVTWLVNSATHAFGYRNFNCPDLSRNCWWVAVLSFGEGWHNNHHAHPASARHGLRWFEVDITWLHIRLLQKLGLTRRVRQARYPG

SEQ ID NO: 177 - protein, 260 aa, Synechococcus sp.
MVVLHVSAIVALLPRFWSWQAVLTLGILYWATACLGVTIGYHRLLSHRSFRVPKWLERFFATCGALSCQQGPITWAGLHRHHHKFSDTDADHHNSHRGFWWSHMGWMFETIPAMQAIPRLSGDLSSDPYYRWLNRNFLYLQFPLAFLLFVIGSITGAGGWALVLWGIPLRLVLVYHVTWLVNSATHCWGTIAYDSGDASRNNKWVAALTFGEGWHNNHHAFPHSACHGLQKGQIDLTWEHIRLMRALGLAKNIRLPIKPA

SEQ ID NO: 178 - protein, 287 aa, Pseudanabaena biceps
MTATTEAISTELKLNWTNVAFFSAFHLAALAAPFYFSWQAVILTVFLHWLFGSIGITLGYHRLLSHRSFQVPQWLEYIIATIGALALQGGPVFWVGGHRQHHGFTEDNQKDPYSANKGFWWSHMMWIMYSKPEHFQRSNYSKFAPDLANDPYYKFLDVYFLLLQVPLAALIYIIGEQCFSGLGMSFLVYGVAVRSVALWHSTWLINSACHKWGYKNFTENPDHSTNLWWAAILTYGEGWHNNHHAFPKSARSGLKWWEIDPTWGVINLLQSVGLAKKVYLSEPWQAK

SEQ ID NO: 179 - protein, 303 aa, Synechococcus sp.
MVTSTPADASTQRELRIRAAVMAPRQMLPSHQRRLKGGTTSFMVVIHVLATVALLPRFWSWQGLVAFGVLYWTTVLGVTLGLHRLVAHRSFEVPGWLERVLVVMGTLACQSGPIDWVALHRHHHRFSDQPNDHHDAGRGLWWSHSEWMLHDIPALKEKHRYAGDLLSDRFYVWLDRWFLVLQIPLGLALYWYGETAGVHGGGVGLVLWAIPLRLAVVYHVTWLVNSATHAFGYRNFNSPDLSRNCWWVAVLSFGEGWHNNHHAYPNSARHGLRWFEFDITWMHIRLLRRLGLTRKVRQARYIG

SEQ ID NO: 180 - protein, 298 aa, Synechococcus sp.
MTTTSLNSNQKPTNQPDNQESLQLNWTTTIFYVVMHAIALLGFGYFSWQALGLTIFLHWLTGSIGICLGYHRLLSHRSFQVPKWLEYIFATLGALALQGGPVFWVGGHRQHHGFTEDNDKDPYSASKGFWWSHLAWILYSKKEHFDFKNYYSFAPDLGSDTYYRFLDKYFLSLQIPLGILLYFIGATQGQGIGFVIYGIFVRAVLVSHSTWFINSACHKWGYRNFGDSDDHSTNLWWAALVTYGEGWHNNHHAYPKSARAGLNWWEIDPTWGAIWLLRAVGLAKKVYIPAPWLPAGLK

SEQ ID NO: 181 - protein, 302 aa, Cyanothece sp.
MTSHLSPHSLAGGNLVMTKELENSRQPALKWNWTNVGFFAVFHALALLAPWFFSWSALGVVLVLHWLLGSIGICLGYHRLLSHRSFNVPRPLEYAIALLGTLALQGGPIFWVGNHRQHHAHTEDDIKDPHSSHRGFWWSHMLWIVAPQPQTLDRDHYGRYAPDLMRQPFYRWLDRYFLLLQIPLAVLLYGLGGWSFVVYGVFVRAVLLWHSTWLVNSATHQWGYRNFASKDTSRNLWWVSLCTYGEGWHNNHHAFPRSAQSGWHWWELDPTWWVIQLLQVLKLADKVKTFPRSLANPCSPQD

SEQ ID NO: 182 - protein, 284 aa, Microcoleus sp.
MTSKTLAAVPDSRSDLKFNWVSATFFVAIHALAMLAPWFFSWSALGVCLFLHWLFGSIGICLGYHRLLTHRSLQVPKWLEYIITTIGTLALQGGPIFWVAGHRQHHAHPEDVVKDPYSAKRGFWWSHMLWLVYQTPGLFDRESYKKYAPDLARDPYYCWLDRYFLLLQIPLGLLLYALGGWSFVIWGMFARAVLLWHSTWLINSATHMRGYQTYDVEDGSRNLWWAAILTYGEGWHNNHHAQPNVAPAGRQWWEVDMTWWAIQVLKTLGLAKKVVMPPQREVQA

SEQ ID NO: 183 - protein, 276 aa, Fischerella sp.
MTTHSTAAVDKQPLTLSWTSVVFFGTFHALALLAPWCFSWSALGITIFLHWLFGSIGICLGYHRLLTHRSLHVPKWLEYAIATLGALALQGGPIFWVAGHRLHHLHTEDIDKDPYSSRRGFWWSHMLWIFYPRPEFFEYEMYKKFASDLDRDPFYRWLNRYFLLLQIPVAVLLYALGGWSFVVYGVFLRAVLLWHSTWLINSASHLRGYRHFQVNDNSRNLWWAALLTYGEGWHNNHHAHPNLAKAGLSWWEVDMTWWAIKVLQTLGLAKKVNVNS

SEQ ID NO: 184 - protein, 283 aa, Chamaesiphon minutus
MTANLLEKREHSSQQQETAPLSWTNVFFFGSFHLIALAAPWYFNWSALGTAILLHWLFGSIGICLGFHRLLTHRSFQVPKPLEYVFATLGALALQGGPIFWVAGHRQHHLYTEDWDKDPYSAKRGFWWSHMLWIFYPKSNFFNLDEYKKAAPDLAKDAYYRWLDKYFLFLQIPVALLLYAIGGWSFVIYGVFVRAVILWHSTWLINSASHITGYRSHESDDNSRNLWWAALLTYGEGWHNNHHAYPNVAKGGWQWWEIDMTWWSIQLLQKLGLAKRVILPPTN

SEQ ID NO: 185 - protein, 284 aa, Nostoc sp.
MTAKYLAVAPETGNSPRLRWVNVAFFATIHVLALLAPWFFSWSALGLLVFLHWLFGSIGICLGYHRLLSHRSFQVPKWLEYAIALIGALALQGGPIFWIGGHRQHHAHTEDINLDPYSAKRGFWWSHLLWIMYPRSEFFDYEIYKKYAPDLERQPYYRWLDRYFLLLQIPLGILLYALGGWSFVVYGMFVRAVLLWHSTWFVNSASHIWGYRTFDANDEARNLWWVSLLTHGEGWHNNHHTFPHVAKAGFQWWEIDITWWSIKTLKALGLAKKVILPPAQARQR

SEQ ID NO: 186 - protein, 280 aa, Cyanothece sp.
MTSNFLEAPTVGEQSLRVNWVSVGFFGAAHAVVLLAPWFFSWSALGIAIFFHWLTGSIGICLAYHRLLTHRSFQVPKWLEYILAMIGGLALQGGPIFWVAGHRLHHAHTEDIDKDPYSAQRGFWWSHILWLFYDRKEFFDYETYKRFAPDLARDPFYRWLNRYYLLLQIPVGFVLYLLGGWSFVIYGIFVRAVLLWHSTWLINSATHMWGYRTFDSKDNSRNLWWAAIITYGEGWHNNHHAHPNVVKAGLKWWEIDMTWWAIQTLKSLGLAKKLVLPMAN

SEQ ID NO: 187 - protein, 285 aa, Nostoc sp.
MTAKNLAIASEGETPLRLRWINVVFFGTIHALALLAPWFFSWQALGLLVFLHWLFGSIGICLGYHRLLSHKSFQVPKWLEYAIAIIGALALQGGPIFWVGGHRQHHAHTEDINLDPYSAKRGFWWSHMLWILYPRYEFFDEETYRKYAPDLARQPFYRWLDRYFLLLQIPMGLLLYALGGWPFVFYGMFLRAVLLWHSTWFVNSATHMWGYRTFDADDNARNLWWVSLVTYGEGWHNNHHTYPHVAKAGFQWWEIDVTWWSIKLLQTLGLAKKVILPPPLNTTQG

SEQ ID NO: 188 - protein, 275 aa, Coleofasciculus chthonoplastes
MPPQSRTNLPFHWPSLLFFGTIHALALLAPWFFSWSALGVMILLHWLFGSIGICLSYHRLLTHRSFQVPKWLEYILTTIGAVALQGGPIFWVAGHRQHHAHPDDANKDPYSARHGFWWSHMLWLFYHKPEFFDYEVYQKYAPDMARDAYYRWLDRYFLLLQLPLGLLLYVVGGWQFVFWGVFVRAVVLWHSTWLINSATHLRGYQTYEVNDGSRNLWWAAILTYGEGWHNNHHAHPNVAPAGRKWWEIDMTWWAIQLLQGVGLAKKVVLPPQKAA

SEQ ID NO: 189 - protein, 291 aa, Leptolyngbya sp.
MISSPAGAALAPYNTQPTQQFSWISLGFFSAFHLIALVAAPFFFSWSALGVAVLLHWLFGGIGICLGYHRMLSHRSFQVPRWLEYAIAFLGALSIQGGPIFWVAGHRRHHAFTEDVAADPYSAKRGFWWSHVLWLIYLQADTFDQKRYGKYAPDLMRQPFYRWLNRYFLLLQLPLAAGLYALGGWSWVIYGIFVRAVFLWHATWFVNSATHRWGYRTFAAKDNSRNLWWVSLLTYGEGWHNNHHAHPRAAQTGWRWWEVDVTWWTIQLLRRLGLAKRVVAMTPQGTTRILR

SEQ ID NO: 190 - protein, 285 aa, Acaryochloris marina
MTTNPVVTASAGRPALSWINVGFFGAFHLLAISLAPHYFSWSALGVALFLHWLLGGIGICLGYHRLLSHRSFRVPKVLEYAIATLGSLAIQGGPIFWVSGHRQHHAHTEHVNDDPYSAQRGFWWSHILWIFYPRPEYFDAKHYRKNAPDLARQPFYRWLDRNFLMLQIPLAAVLYALGGWSFVVYGIFVRAVILWHSTWLVNSASHMWGYRTFESDDGAQNLWWVSLLTYGEGWHNNHHSYPHVAKSGYRWWEIDLTWWAICALKQMGLAKKIVTYPKSLQYVRK

SEQ ID NO: 191 - protein, 278 aa, Rivularia sp.
MTTRSAVTEGKQPLALSWTFTLFIGSIHVLALLAPWFFSWSALGVMIFLHWLFGSIGICLGYHRILSHRSLQVPKWLEYIIAIIGAMAIQGGPIFWVAGHRLHHAHTEDEDKDPYSAKRGFWWSHMLWMIYPVEEFFDYEQYKKYAPDLVRQPFYRWLNRYFILLQIPLVALLYALGGWSFVIYGVVLRAVLLWHSTWLINSATHIRGYRTFDSEDNSRNLWWAAILTYGEGWHNNHHAHPNVAKAGLRWWEIDMTWWAIKVLQSLGLAKKVVMPVNE

SEQ ID NO: 192 - protein, 284 aa, Leptolyngbya sp.
MTENSVQKNTATKAPLNWTTVIFFTLVHILALLAPWYFSWQALGITLLLHWLFGSIGICLGYHRLLSHRSFQVPKWLEYLIALVGTLALQGGPIFWVAGHRLHHAHTEDEQEDPYSAKRGFWWSHMLWMLYPEAKFFDSDLYSRYAPELARDPVYRWLNRNFILLQIPLGLLLYGLGGWSFVIYGIFLRAVLLWHSTWFINSVTHMWGYRTFTTEDNSRNLWWAAIFTYGEGWHNNHHAYPNVARAGWQWWEIDVTWWAIWVLQTLGLATKVVKIPAHVSPTLK

SEQ ID NO: 193 - protein, 283 aa, Oscillatoriales cyanobacterium
MTSNRFDTDLQAGQSQSFALNWTNILFFATFHLIALSAPWFFSWSALGVALCLHWLFGSVGICLGYHRLLTHRSFQVPKPLEYAIAVIGSLALQGGPIFWVAGHRKHHLYTEDIDKDPYSARRGFWWSHMLWLFYKRADVFDHNAYRQYAPDLDRDVFYRWLDRYFLLLQIPLGVVLYLLGGWSFVLYGVFVRSVLLWHSTWLINSATHMTGYRTYEPADNSRNLWWAALLTYGEGWHNNHHAYPNVAKAGWKWWEIDMTWWVIAALKRLGLARKVVLPPVQS

SEQ ID NO: 194 - protein, 283 aa, Oscillatoria acuminata
MQLESMPNSPENQSKLNWIITGFFIAIHCLALLAPWCFSWSALGVMLLLHWFLGSIGICLGYHRLLSHRSFQVPKWLEYTIALIGASALQGGPIFWTAGHRLHHAYTEDEIKDPYSARKGFWWSHLLWMIYPRPEFFNKELYNRFAPDLARDPFYCWLDRYSLLMQIPLVVVLYLLGGWPFIVYGVFVRIVLLWHSTWLINSVTHMWGYKTFPTSDNSRNLWWAAIFTYGEGWHNNHHAYPNVAKAGWRWWELDMTWWAIWVLKTVGLAKKVVMPPTPKANQG

SEQ ID NO: 195 - protein, 279 aa, Thermosynechococcus elongatus
MTSSLELQPQTSRLNWGFVLFLGAVHILAAVALFFFSWSALAVTIFLHWLFGSIGICLGYHRLLSHRSFQVPQWLEYVIAVVGALAMQGGPIFWVAGHRLHHAHTEDEIKDPYSARRGFWWSHMLWLVYPQSQFFNAEEYARFAPDLTRDPFYRWLDRYFLLLQLPLALLLYGLGGWSWLLWGMFMRAVFLWHSTWLINSATHKWGYRRFETEDNSRNLWWAALLTYGEGWHNNHHAYPHVAKAGWYWWEVDPTWWVIRTLQGLGLAAKVQLPPPKALS

SEQ ID NO: 196 - protein, 297 aa, Synechococcus sp.
MAVSLPTQSLDNPNRFASLAQPEPQKSFRFAWEFVLFFVVFHALALVGGIVCFSWPALGVALLLHWCFGSLGICLGYHRLLSHRSFQVPKWLEYVFATFGALAIQGGPIFWVAGHRQHHAFTEDEERDPYSARKGFWWSHMLWLIYQREEFFNREKYSAFAPDLARDPFYRWLDKYGLLLQLPLAGVLYLLGGWPFVIYGIFVRTVLLLHCTWLINSASHFWGYRTFESDDNARNLWWAALLTYGEGWHNNHHADPKCVKAGLRWWEIDMTYWVIWVLARLGLARKLHLPAQSQPTR

SEQ ID NO: 197 - protein, 282 aa, Chroococcidiopsis thermalis
MTIGSVAEGKQPLALSWINVVFFGTFHVVAMLAPWCFSWSALGVTIFLHWLFGSIGICLGYHRLLSHRSLQITPKWLEYAIAFIGALAMQGGPVFWVAGHRQHHAHTEDVDKDPYSSKRGFWWSHMLWILYPRSEFYIYDNYRKFAPDLDRDPYYRWLDRYFLLLQIPLAVLLYLLGGWSFVIYGVFLRAVLLWHSTWLINSATHLYGYRHFEVEDNSRNLWWAALLTYGEGWHNNHHAQPNVAPAGRRWWEIDMTWWAIQALQALGLAKKVVLPPTQKAKA

SEQ ID NO: 198 - protein, 282 aa, Cyanothece sp.
MTSNFTQAPVDGEQSLKLHWVSIMFFGAAHLLALLAPWFFSWSALGVMLFFHWLTGSIGICLAYHRLLSHHSFQVPKWLEYILAMIGALALQGGPIFWVAGHRLHHAHTEDIDLDPYSARRGFWWSHILWLFYDRKEFFDYETYKRFAPDLARDPFYRWLNQYYLFLQIPLGLLLYVIGGWSFVIYGIFLRAVMLWHCTWLINSATHMWGYRTFNSEDNSRNLWWAALLTYGEGWHNNHHAHPHVVKAGLKWWEIDMTWWAVQALKSLGLAKKLVLPSVKRA

SEQ ID NO: 199 - protein, 280 aa, Geitlerinema sp.
MKPQLEFDSSETLKLDWKTVLFFAAVHGLALLAIPFFSWSALGVMLGLHWLFGSIGICLGYHRLLSHRSFRVPRWLERSIALLGALALQGGPIFWVAGHRLHHAYTEDEQKDPYSARRGFWWSHVLWIFYPRSQFFDYEEYQRFAPDLARDPFYRWLNRYFFWLQFPLALLLYFVGGWSFVVYGIFVRAVFLWHTTWLINSVTHLWGSRAFDCRDNSRNLWWAALLTYGEGWHNNHHAYPQVARCGWRWWEIDVTWWAIALLQRLGLATAVQNPPTSASR

SEQ ID NO: 200 - protein, 279 aa, Thermosynechococcus vulcanus
MTSSLELQPQTSRLNWGFVFFLGAVHILAAVALFFFSWSALAVTIFLHWLFGSIGICLGYHRLLSHRSFQVPQWLEYVIAVVGALAMQGGPIFWVAGHRLHHAHTEDEIKDPYSARRGFWWSHMLWLVYPQSQFFNAEEYARFAPDLTRDPFYRWLDRYFLLLQLPLALLLYGLGGWSWLLWGMFMRAVFLWHSTWLINSATHKWGYRRFETEDNSRNLWWAALLTYGEGWHNNHHAYPHVAKAGWYWWEVDPTWWVIRTLQGLGLAAKVQLPPPKALS

SEQ ID NO: 201 - protein, 285 aa, Crinalium epipsammum
MTSNSMGTIQSSSEQLKLSWVNVGFFGTIHALALLAPWFFSWSALGVMIVLHWLFGSIGICLGYHRLLTHRSLQVPKWLEYTLATIGALALQGGPTFWVAGHRQHHLHTEDRDKDPYAASRGFWWSHMLWIMYPRAEFFERATYNKYAPDLARDPYYKWLDSNFLALQIPVAVLLYLLGGWSFVIYGTFLRAVLLWHSTWLINSASHMTGDRRFEVPDGSRNLWWTAILTYGEGWHNNHHAYPNVAKAGWKWWELDMTWWAIKALESLGLAKRIVLPPAEVIADL

SEQ ID NO: 202 - protein, 286 aa, Anabaena cylindrica
MTLNSSNLLGNGQPLRLHWVSVAFFGTIHAIALLAPWHFSWAAVGVTVFLHWLFGSIGVCLGYHRLLSHRSFRVPKWLEYAIAILGALSIQGGPIFWVAGHRLHHAYTEDEDKDPYSARKGFWWSHMLWIFYPRSEFFDYELYQRFAPDLARDPFYIWLNRYFLLLQIPVALLLYALGGWSFVIYGVFLRSVILWHTTWLINSVTHMWGYRTFASDDNSRNLWWAAILTYGEGWHNNHHAYPHVAKCGWRWWEIDLTWWTIAVLRTLRLATNINLPPDQAMKPGNV

SEQ ID NO: 203 - protein, 268 aa, Synechococcus sp.
MSWASVGFFAAVHGVALLAPWFFSWSALGMAIAFHWLFGSVGICLGYHRLLSHRSFKVPQWLEYIIATIGALAMQGGPTFWVAGHRQHHAFTEHTEKDPYSSARGFWWSHMQWMFYLKPEVFDRRVYAQYAPDIARDRYYAFLDRYFLVLQVPLAFALYAVGGWSFVIYGVFVRAVLLWHCTWLINSANHLWGYRNFESDDNSRNLWWVALLTYGEGWHNNHHAFPKMAKSGLRPWEIDTTWWLIKGLQSAGLATKVVMPPVKARVLS

SEQ ID NO: 204 - protein, 282 aa, Oscillatoria sp.
MTLNDLSISPEERPNLDWIAVAFFTSIHGLALLAPWFFSWSALGVTLFLHWFFGSIGICLGYHRLLSHRSFQVPQWLEYTIAIIGALALQGGPIFWIAGHRLHHAFTEDEIKDPYSARRGFWWSHMLWIFYPYSTFFDYEKYQRFAPDLARDPFYRWLNRNFLLLQIPLALLLYVVGGWSFIIWGMFVRAVLLWHSTWFINSVTHLWGYRSFETNDNSRNLWWAAILTYGEGWHNNHHAHPNVAKAGWRWWEIDITWWAIWLMKSLGLARKVIMPPVPVTDK

SEQ ID NO: 205 - protein, 281 aa, Chroococcidiopsis thermalis
MTTNMVETDRENGQPLVPNWTSITFFAAVHAIALLAPWFFSWSALGVAIFLHWLFGSIGICLGYHRLLTHRSLQVPKGLEYAIALIGSLALQGGPLFWVAGHRRHHAYTEDIDKDPYSAKRGFWWSHMGWLLYKRPEFFEYESNRKFAPDLAKDAFYRWLDRYYLLLQIPLGVGLYLLGGWSFVIYGLFVRAVLLWHSTWLINSASHITGYRSHDSDDNSRNLWWAAILTYGEGWHNNHHAYPNVAKAGWKWWEIDLTWWTILLLKQMGLARKIVLPPTAS

SEQ ID NO: 206 - protein, 285 aa, Leptolyngbya sp.
MTAQLTQPRLDPQPEVKRPPMDWVTLGFFAVFHGVALLAPWFFSWSALGVAILLHWLCGGVGICLGFHRLLSHRSFQVPRWLEYPIAVLGTLAMEGGPIFWVSGHRKHHAFTEDVQNDPYSAKRGFWWSHMLWILYPHRENFDKNYYGKYAPDLMRQPFYRFLNNYFLLFQVLLALLLYAIGGWSFVIYGVFVRAVCLWHATWLVNSATHTWGYRTFHTDDNARNLWWVSLVTYGEGWHNNHHTYPRAAQTGLKWWEIDVTWYTILLFERLGWAKKVKRIPATHR

SEQ ID NO: 207 - protein, 283 aa, Gloeobacter violaceus
MTANVVSGAGGVAGWRALNWPTLGFLVLMHGLALLAPWFFNWQALAVALGLHWLIGSLGICLGYHRLLTHRSFRVPRGLECVFAMLGALALQGGPIFWVAAHRLHHAHADDDARDPHSSGRGFWWSHVLWLLYRHPELSDGQTHRKYAPDLARQPFYRWLNRYHLLLQIPLAVLLFALGGWPFVVYGVFVRAVVLWHTTWLINSAAHLAGYRTSEVPDSSRNLWWAALLTYGEGWHNNHHAQPNAARAGLRWWEVDMTWWVIRALEMAGLATQVVPPRPNPVR

SEQ ID NO: 208 - protein, 293 aa, Nostoc punctiforme
MTANFGAFAKSVGEAIAPERGKSPQLSWTNVVFFTTFHALALMSPWFFSWSALGLLVFLHWLFGSIGICLGYHRLLSHKSFQVPKWLEYAIALIGALALQGGPIFWVGGHRQHHAHTEDINLDPYSAQRGFWWSHILWIFYPRPEFFDYDTYKKYAPDLARQPFYCWLDRYFLLLQIPFALLLYGLGGWPFVFYGVFLRCVLLWHSTWFVNSASHLWGYRTFDANDGARNLWWVSLVTYGEGWHNNHHTYPHMAKSGLSWWEIDVTWWSILVLQTLGLAKKVVSRPPQGATHG

SEQ ID NO: 209 - protein, 272 aa, Leptolyngbya sp.
MVNTAALPTPRLRWTVVVSTVILHLLALLALLPSNFSWRAIGIAIFLHALTQGLGISLGFHRMASHRSLRVPLWLEHLFILLGTLAFQGGVYGWVGYHRLHHKHTDQALDPHSASKGFWWSHINWLMHTVPTHSLLPRMTQDIRDDKFYQFCHNHYITLQVALGLLLYLMGGMPCLVWGIFVRLLVSFHGTCFVNSACHKFGYRSTDTDDMSTNCWWVAILTYGEGWHNNHHACQSSACFQKSWWEIDPVWQVIRLLQALGLAEKVKTASPS

SEQ ID NO: 210 - protein, 294 aa, Synechococcus sp.
MTTSFSRQSLNHPAPLIQPEPQESFRFAWEHVLFFAIFHVVALVGGIFFFSWPALGVALFLHWCFGSLGICLGYHRLLSHRSFQVPKWLEYVFTTLGALAVQGGPIFWVGGHRQHHAFTEDEEKDPYSARKGFWWSHILWLIYHREEFFNPEKYSAFAPDLARDPFYRWLDRYALLLQLPLAAILYLLGGWSFVVYGIFVRTVLLLHCTWLINSASHFWGYRTFESNDNARNLWWAAILTYGEGWHNNHHADPKCVKAGLRWWEIDMTYWAIWLLARLGLARKLHLPAQGQAAR

SEQ ID NO: 211 - protein, 285 aa, Nostoc sp.
MTANFGAIAPERGNSPQLRWINVVFFGVFHALALLSPWFFSWSALGLLVFLHWLFGSIGICLGYHRLLSHKSFQVPKWLEYAIALIGALALQGGPIFWVGGHRQHHAHTEDIDLDPYSAQKGFWWSHILWIFYPRPEFFDYDTYKKYAPDLARQPFYCWLDRYFLLLQIPFALLLYVLGGWPFVFYGVFLRCVLLWHSTWFVNSASHLWGYRTFDADDGARNLWWVSIVTYGEGWHNNHHTYPHMAKSGLFWWEIDVTWWSIQLLQTLGLAKKVVSSPPQGATHG

SEQ ID NO: 212 - protein, 277 aa, Cyanothece sp.
MTKTIKISPDGGQPLTLNWVAVGFFSAVHLVALSAPWFFSWSALGVAIFLHWFIGSIGICLGYHRLLTHRSFEVPKALEYIIAIIGALALQGSPIFWVAGHRMHHAYTEDMKKDPYSAKRGFWWSHMLWLFYDRQEFFNYDQYKKYAPDLDKQPFYRWLDRYFLLLQLPLGLFLYFLGGWSFVIYGLFVRTVLLWHSTWFINSASHLRGYRTYQSNDNSRNLWWAAILTYGEGWHNNHHAYPNIAKAGLKWWEIDVTWWTIKILSKLNLAKKIKLPV

SEQ ID NO: 213 - protein, 277 aa, Cyanothece sp.
MTNTIKISPDKRKPLTFNWIAIAFFSAVHLVALSAPWFFSWSALGVAIFLHWLIGSIGICLGYHRLLTHRSFEVPKPLEYIIAIIGALALQGSPIFWVSGHRMHHAYTEDMEKDPYSAKRGFWWSHILWLFYDRPEFFNYDQYKKYAPDLDKQPFYRWLDRYFLLLQLPLGLLLYLLGGWSFVIYGLFVRTVLLWHSTWFINSVSHLIGYRTYNSNDNSRNLWWAAILTYGEGWHNNHHAYPNIAKAGLKWWELDVTWWAIKILSKLNLAKKIKLPV

SEQ ID NO: 214 - protein, 277 aa, Cyanothece sp.; identical to SEQ ID NO: 213

SEQ ID NO: 215 - protein, 280 aa, Synechococcus sp.
MTQSVQHPTQKPTLDWVAVFFFGAVHLLALGLAPFFFSWSAIAVCLFLHWLFGSIGICLGYHRLLSHRSLKVPQWLEYTLAFIGALALQGGPIFWVAGHRLHHAHTEDEVKDPYSAKRGFWWSHMLWIFYPDQQFFDADQYSRYAQDLAKQPFYRWLNRNFLLLQIPLAIALYLLGGWSWVVYGMLVRAVLLWHSTWFINSVTHVWGYRSHASDDNSRNLWWAAILTYGEGWHNNHHAYPSVAKAGWRWWEIDVTWWAIWLLQRLGLATKVILPPAVVTE

SEQ ID NO: 216 - protein, 274 aa, Stanieria cyanosphaera
MTQTAISPERKSQLVLSWKSVALFSTIHALALLAPWFFSWSALGVMIFLHWLFGSIGICLGYHRLLTHRSFQVPQWLEYILATIGSLALQGGPIFWVAGHRIHHAYTEDEEKDPYSAKKGFWWSHMLWLLYPREEFFSYESYKKFAPDLDRDPYYRWLNKNFILLQVILGLILYGLGGWSFVIYGIFLRAVLLWHSTWLINSASHRDGYQNFPVEDNSRNLWWAAILTYGEGWHNNHHAHPRVAKAGQRWWEIDVTWWAIKALQMLGLAKKIVV

SEQ ID NO: 217 - protein, 279 aa, Calothrix sp.
MTAKFMASAPETGNSPRLSWTNVVFFTTFHALALLAPWFFSWTALGLLVFLHWLFGSIGICLGYHRLLSHRSFQVSKGVEYAIAFIGALALQGGPIFWVGGHRQHHAHTEDIDLDPYSAQRGFWWSHMLWIFYPRSQFFDYDTYQKYAPDLARQPYYRWLDRYFILLQIPLGLLLYALGGWSFVIYGIFVRAVLLWHSTWFVNSATHKWGYRSFNANDGSRNLWWVSIVTYGEGWHNNHHTYPHVAKAGFSWWEVDVTWWSIKALKTLGLAKKVVMPPS

SEQ ID NO: 218 - protein, 288 aa, Pseudanabaena biceps
MTRLFSDVIPAPSNPNPEPLKLSWTSIAFFSIFHILALLAPWYFSWSALGVTILLHWFLGSIGICLGYHRLLSHRSFHVPKWLEYAIALVGAAALQGGPIFWIAGHRLHHAHTEDMELDPYSARRGFWWSHMLWILYPKPDFFNLDKYGRYAPDLVRDPFYVWLDKYFLLLQIPLGLGLYLLGGWSYIIWGMCLRSVLLWHSTWFINSVTHMWGYRTFETNDNSRNLWWAAIVTYGEGWHNNHHAYPHVAKAGWRWWEVDVTWWAIQMLKSLGLATKVVMPPTLKNQT

SEQ ID NO: 219 - protein, 263 aa, Nodularia spumigena
MAFFAAFHALALLAPWFFSWPALGLLVFLHWLFGSIGICLGYHRLLSHKSFQVPKWLEYAIATIGALALQGGPIFWVGGHRQHHAHTEDVNLDPYSAQRGFWWSHMLWIFYPRSEFFDYQVYQKYAPDLARQPYYRWLDRYFLLLQIPLALLLYVLGGWSFVIYGTFLRSVLLWHSTWFVNSASHLWGYRTFDADDGARNLWWVSLVTYGEGWHNNHHTYPHMAKSGLSWWEIDMTWWSIKLLQTLGLAKKVVSVPPHSAAIK

SEQ ID NO: 220 - protein, 280 aa, Gloeocapsa sp.
MTTSAVNAESKQPLALSWINVAFFGTVHAVAIAAFWNFSWSALGVTLFLHWLFGSIGICLGYHRLLSHRSLQVSPKWVEYAIATCGALAIQGGPIFWVASHRLHHAHTEDNDKDPYSSRRGFWWSHMLWIFYPRREFFEFGNYKRFAPDLARDPYYNWLDRNFLLLQIPLGIVLYLCGGWSFVIYGIFVRSVILWHTTWLINSATHLRGYQNFPVDDNSRNLWWAAILTYGEGWHNNHHAYPNVAKAGQRWWEVDMTWWAIKLLQTAGLAKKVVMPVTKA

SEQ ID NO: 221 - protein, 285 aa, Cylindrospermum stagnale
MPAKFLAIAPEGGQALRLSWTNVAFFAAVHALALLAPWFFSWSALGLLLFLHWLFGSIGICLGYHRLLSHRSFQVPKGLEYAIAFIGALALQGGPISWVGAHRQHHAHTEDIHLDPYSAQRGFWWSHILWILYPRPEFFDYQIYQKFAPDLARQPFYRWLDRYFLLLQVPLAVLLYALGGWSFVIYGMFVRTVLIWHSTWFVNSASHIWGYRTFESNDGARNLWWVSLVTYGEGWHNNHHTYPHMAKSGLRWWEIDVTWWSIKALQTLGLAKKVISSPPQGAGQG

SEQ ID NO: 222 - protein, 285 aa, Anabaena variabilis
MTVKHLAIAPEGKKSLRLNWTNVAFFTTVHALALLAPWFFSWSALGLLLFLHWLFGSIGICLGYHRLLSHKSFQVPKWLEYAIATIGALAMQGGPIFWIGGHRQHHAHTEDVNLDPYSSQRGFWWSHMLWILYPRSEFFDYEIYQKYAPDLARQPFYRWLDRYFLLLQIPFGLLLYAIGGWSFVIYGVVLRAVLLWHSTWFVNSATHMWGYRTFNADDNARNLWWVSIVTYGEGWHNNHHTFPNVAKAGFQWWEVDVTWWSIKLLQTLGLAKKVVLPPSQRMNQG

SEQ ID NO: 223 - protein, 278 aa, Synechocystis sp.
MTASVITEGKPPLALSWISIGFFAAFHVLAVLAVGCFSWSALGVTLFLHWLFGSIGICLGYHRLLSHRSFQVPKLLEYAIAIIGALAIQGGPIFWVAGHRLHHAHTEDEEKDPYSSRRGFWWSHIVWLFYPRPEFFDYKQYSKFAPDLARDPFYRWLNRYHLFLQIPLAGLLYVCGGMPFVVYGIFLRSVLLWHSTWLINSATHIFGYRNFPDSKDDSRNLWWAAILTYGEGWHNNHHAYPKVAKAGLRWWEIDVTWWAIKTLQILGLAKKVTMPPTR

SEQ ID NO: 224 - protein, 285 aa, Nostoc sp.
MTVKHLAIAPEGKKSLRLSWTNVAFFTTIHALALLAPWFFSWSALGLLLFLHWLFGSIGICLGYHRLLSHKSFQVPKWLEYAIATIGALAMQGGPIFWIGGHRQHHAHTEDVYLDPYSSQRGFWWSHMLWILYPRSEFFDYEIYQKYAPDLARQPFYRWLDRYFLLLQIPFGLMLYAIGGWSFVIYGVVLRAVLLWHSTWFVNSATHMWGYRTFNADDNARNLWWVSIVTYGEGWHNNHHTYPNVAKAGFQWWEVDVTWWSIKLLQTLGLAKKVVLPPSQRMNQG

SEQ ID NO: 225 - protein, 236 aa, Synechococcus sp.
MHWFFGSIGICLGYHRLLSHRSFQVPKWLEYAIAFIGALALQGGPIFWVAGHRLHHAHTEDEQEDPYSAKRGFWWSHMLWMVYPDDKFFDYTQYHRYARDLAKDPFYRWLNRYFIFLQIPLGLVLYALGGWSFVIYGIFLRTVLLWHSTWFINSVTHMWGYRTFESKDNSRNLWWAAIVTYGEGWHNNHHAYPNVARAGWRWWEIDVTWWSIWLLKSLGLAHKVVKIPAHIKSSVK

SEQ ID NO: 226 - protein, 277 aa, Leptolyngbya sp.
MTKAISKNTQKRPLDWVSVFFFAAVHGLALLAPWFFSWSSLGIAIFLHWLFGSIGICLGYHRLLSHRSLQVPKWLEYTIAFIGTLALQGGPIFWVAGHRLHHAHTEDETQDPYSAKRGFWWSHMLWMLYPTPDFFEYKRYKKYAPDLVRQGYYRWLNRNFLLLQIPLGLLLYVLGGWSWVIYGIFVRAVILWHSTWLINSATHVWGDRRFDVDDNSRNLWWAAIFTYGEGWHNNHHAYPRVAKAGWRWYEIDMTWWAIWLLSRLGLAKKVIMPPPNL

SEQ ID NO: 227 - protein, 284 aa, Geitlerinema sp.
MSVQLSSSASTGEQSLVLSWSNVAFFSAFHILALLAPWFFSWSALGVMLFLHWLFGSIGICLGYHRLLSHRSLRVPKPLEYTFGLLGALALQGGPIFWVAGHRLHHAFTEDVHKDPYSARRGFWWSHILWILYPRPEFFDVNTYRKNAPDLARDPFYTWLDRYFLLLQIPLAGLLYLLGGWPFIVYGVFVRVVLLWHTTWLINSVTHLFGDRTFETEDGSRNLWWAALLTFGEGWHNNHAYPRVAKAGWKWWQIDITWWVIQVLITLGLAKKVIMPPLEADTTR

SEQ ID NO: 228 - protein, 277 aa, Gloeocapsa sp.
MISDSIQIAPKPQQPLKLSRSNLSLFVAIHALALLSPWFFSWSALGVMLFLHWLCGSIGICLGYHRLLTHRSFKVPKPLEYIITTIGALALQGGPIFWVSGHRLHHAHTEDIDQDPYSAKRGFWWSHMLWLIYPRQEIFNYETYKRFAPDLERDPYYRWLNQNFIILQVALGLLLFALGGWSFVIYGIFLRSVLLWHSTWLINSATHIRGYRTFDTEDNAGNLWWAALLTYGEGWHNNHHAHPRVAKAGLKWWEIDVTWWAIKVLQSLSLAKNVVMP

SEQ ID NO: 229 - protein, 340 aa, Leptolyngbya sp.
MTQIPSSAPNTRDGLVFHWPTVLFIALIHGLALGLAPFFFSWSAVALTLGLHWLFGSIGICLGYHRLLSHRSFRVPQGLEYAIALIGVLAFQGGPIFWVAGHRLHHAYTEDRDRDPYSAQRGFWWSHMLWLFYQRSTFFDPQQYYRYAPDLARDPVYRWFDSYGALLQIPLAGLLYLLGGWPFVVYGMLVRSVLLWHSTWLINSVTHLWGNRPFLCDDNSRNLWWAAILTYGEGWHNNHHAYPQVAPAGWHWWQIDITWWAIRTLQALGLARKVVMPPPERLGHDFRGWDSPLKPLRRLGSILPTYPHSPGGTQGNSHQSSPIGSNADYNADYDKESALP

SEQ ID NO: 230 - protein, 275 aa, Pleurocapsa sp.
MTKQLITPESKQELTLSWASVAFFTVIHALALLAPWFFSWSALGVAIALHWLFGSIGICLGYHRLLTHRSLQVPQWLEYVIATIGAMALQGGPIFWVSSHRLHHAYTEDEQKDPYSAKRGFWWSHMLWLLYPREAFFNYESYKKYAPDLDRHAYYRWLNRNFLWLQIPLGLLLYGLGGWSFVIYGMFLRAVLLWHSTWLINSATHVRGYRNFPVEDGSRNLWWAAILTYGEGWHNNHHAHPRIAKAGQRWWEIDPTWWVIQGLKMLGLAKNIVTS

SEQ ID NO: 231 - protein, 284 aa, Cyanothece sp.
MTANLPIALPETPEPLQLNWVAVIFFSTIHVLAVLCCWLFTWPALALTLILHWFLGSIGICLGYHRLLSHRSFQVPRWLEYTIAVIGALALQGGPIFWVAGHRLHHAFTEDELKDPYSARRGFWWSHMLWILYPRKEFFDTAVYERYAPDLARDSFYRWLDRYFLLLQLPLAILLYLLGGWSFVVYGVFLRCVLLWHSTWMINSVTHMWGYRTFETSDNSRNLWWAAIFTYGEGWHNNHHAHPNVAKAGWRWWELDVTWWAIRTLDLLGLASKVVMPPAQTCQH

SEQ ID NO: 232 - protein, 278 aa, Xenococcus sp.
MTQQLTATEGKPVLTLSRTNVAWFAAIHALALLAPWFFSWSALGVMILLHWLFGSIGICLGYHRLLTHRSFQVPQWLEYILATIGSLALQGGPIFWVSGHRLHHAHTEDEEQDPYSARRGFWWSHMLWLCYPREPFFDYESYKKFAPDLDRHPYYRWLNRNFILLQVLLGLLLYGLGGWSFVIYGIFLRAVLLWHSTWLINSATHRWGYRNFPAEDGSRNLWWAAILTYGEGWHNNHHAHPRVAKAGQRWWEIDVTWWAIQILQLLGLAKKIVMAKEK

SEQ ID NO: 233 - protein, 293 aa, Pseudanabaena sp.
MTSTSSQEPFKMRWQDLHWVSVAFFSIVHIAAAIGAPFLFTWKALGVMIILHWMLGSLGICLGYHRMLSHRSFDVPKWLERVFVTLGAMAIEGGPIFWAGTHRMHHGFTEDTERDPYSAKRGLWWSHMGWILRAREDHFGLESNRRFTQDLINDPYYLFLDQNFLLLQIPLAAILFLIGQTQGQGLEFMFYGVVVRAVFLWHSTWLINSACHKWGYRNFADADDHATNLWWAAILTYGEGWHNNHHMYPKSAKAGLNWWEIDLTWQAIRFLNFLGLAKNIHIDKPWQSKPAKS

SEQ ID NO: 234 - protein, 276 aa, Oscillatoria nigro-viridis
MTAKLLPTNPTRYPLDWTAVFFFTTIHALALLAPWCFSWSALGMTLFLHWLFGSIGICLGYHRLLSHRSFQVPKGLEYAIALIGATALQGGPIFWVAGHRLHHAYTEDVDKDPYSARRGFWWSHMLWIFYPNSEFFDYEQYQRFAPDLARDRFYCWLNRNFLLLQIPLGLLLYALGGWSFVIWGMFVRAVLLWHSTWFINSVTHLWGYRTFDTNDNSRNLWWAAVLTYGEGWHNNHHAHPNVAKAGWRWWEYDPTWWAIWLLKRLGIAQKVVMPPT

SEQ ID NO: 235 - protein, 283 aa, Calothrix sp.
MTVKSNSHATTGGDSGAIAQPLNWTYIIFFAAFHLLALLAPWFFSWQALFTALFLHWLCGSIGICLGYHRLLSHKSFQVPQWLEYAIAICGALALQGGPIFWVGGHRIHHAHTEDVDKDPYSAKRGFWWSHILWLFYPRPEFFNREIYQKSAPDLARQPFYQWLDRHFLLLQVPLALLLYALGGWSFVIYGIFVKSVLLWHSTWFVNSATHTWGYKTFAADDNARNLWWVSVVTYGEGWHNNHHTYPNVAKSGYKWWEIDPTWWSIRALQRLGLAKKVILYKS

(19) United States
(12) Patent Application Publication
(10) Pub. No.: US 2016/0289647 A1
(43) Pub. Date: Oct. 6, 2016

(54) PROKARYOTIC DESATURASES MODIFIED BY DIRECTED EVOLUTION TO REDUCE SATURATED AND INCREASE UNSATURATED FATTY ACIDS IN EUKARYOTES

(71) Applicant: WASHINGTON STATE UNIVERSITY, Pullman, WA (US)

(72) Inventors: John A. BROWSE, Palhouse, WA (US); Shuangyi BAI, Pullman, WA (US); James WALLIS, Moscow, ID (US)

(21) Appl. No.: 15/030,405

(22) PCT Filed: Oct. 29, 2014
(86) PCT No.: PCT/US2014/062863

§ 371 Date: Apr. 19, 2016

Related U.S. Application Data
(60) Provisional application No. 61/898,039, filed on Oct. 31, 2013.

(51) Int. Cl. C12N 9/02; C12N 15/82

(52) U.S. Cl. CPC C12N 9/0071; C12N 15/8247; C12Y 114/19001

(57) ABSTRACT

Transgenic eukaryotic (animal, plant and fungal) cells and organisms that contain decreased levels of saturated fatty acids are provided. The transgenic eukaryotic cells and organisms have been genetically engineered to contain and express heterologous prokaryotic enzymes with enhanced desaturase activity. Products made by or from the cells and organisms are also provided.

### BACKGROUND OF THE INVENTION

1. Field of the Invention

The invention generally relates to eukaryotic cells and organisms with decreased amounts of saturated fatty acids. In particular, the invention provides eukaryotic cells and organisms that have been genetically engineered to contain and express heterologous prokaryotic enzymes with enhanced desaturase activity, as well as products made by or from the cells and organisms.

2. Background of the Invention

Plant seed oils are important sources both of high-calorie food and of fatty acids that are essential for human nutrition. The diversity of fatty acids stored in oilseeds worldwide exceeds 200 different types, varying in acyl chain length, degree of unsaturation, and functional groups. Despite this diversity, the most nutritionally important and abundant fatty acids found in the major commercial food oils are of just a few types: palmitate, stearate, oleate, linoleate and linolenate. In seeds, the majority of these fatty acids are esterified to glycerol backbones to form triacylglycerols (TAG); these storage lipids accumulate in specialized lipid bodies with limited metabolic activity. These same few fatty acids also predominate in the membrane lipids of all plant tissues, where their essential roles in metabolism are well established.

Worldwide production of edible fats and oils is dominated by vegetable oils, accounting for nearly 85% of the total. Consumption of fats and oils has important effects on human health that depend largely on their fatty acid composition. Of the five most abundant fatty acids in plants only palmitate (16:0) and stearate (18:0) are saturated hydrocarbon chains with no double bond between carbon atoms. It is well-accepted that a diet high in saturated fats raises the risk of cardiovascular disease, and the incidence of both type 2 diabetes mellitus and insulin resistance is increased by consuming high levels of saturated fatty acids. Since cardiovascular disease and diabetes are major worldwide health problems, dietary guidelines recommend replacing saturated fatty acids with unsaturated fats.

It would be a boon to develop ways to reduce the levels of saturated fatty acids in cells, and/or to increase the levels of unsaturated fatty acids in cells, particularly those that are used as a food source e.g. by humans.

### SUMMARY OF THE INVENTION

The invention provides recombinant desaturase enzymes (herein designated as DES9*) that, when expressed in heterologous eukaryotic cells and/or organisms, cause a reduction in the proportion of saturated fatty acids to unsaturated fatty acids in the eukaryotic cells/organisms and/or an increase in the proportion of unsaturated fatty acids. Exemplary recombinant desaturases are prokaryotic enzymes with amino acid sequences that are modified to enhance their expression and/or activity in eukaryotes. According to the invention, non-human eukaryotic cells/organisms of interest (e.g. those that are used as a food source, that make a product that is used as a food source, or are used as a source of biofuel) are genetically engineered to contain and express nucleic acids that encode one or more of the recombinant desaturase enzymes described herein, with exemplary desaturases being those that desaturate 16:0 saturated fats. Accordingly, (transgenic) eukaryotic host cells and organisms that are genetically engineered in this manner, and products produced by or from such host cells and organisms, are provided, as are methods for making and using the same

It is an object of this invention to provide a method of modulating fatty acid content or composition profile in a eukaryotic host, the method comprising genetically engineering the eukaryotic host so as to contain and express a DNA molecule encoding a recombinant prokaryotic desaturase, wherein desaturase activity of the recombinant prokaryotic desaturase is at least 1.5 fold higher than that of a corresponding native prokaryotic desaturase and wherein at least one of a level of one or more saturated fatty acids is decreased or a level of one or more unsaturated fatty acids, such as mono-unsaturated fatty acids is increased in said eukaryotic host, or both. In some aspects, the recombinant prokaryotic desaturase is a genetically engineered mutant form of a cyanobacterial desaturase, and the cyanobacterial desaturase may be glycerolipid Δ9-desaturase (DSG) from Synechococcus elongatus (Anacystis nidulans, PCC 6301). In additional aspects, an amino acid sequence of the recombinant prokaryotic desaturase includes a mutation at one or more of the mutations listed in Table 2 below. One or more of the mutations may include Arg or Lys at one or both of positions 69 and 240. In other aspects, the recombinant prokaryotic desaturase may comprise one or more eukaryotic sequences, with addition of one or more eukaryotic sequences including an endoplasmic reticulum retention sequence.

Other aspects of the invention provide a nucleic acid molecule comprising nucleotide sequences which encode and express a recombinant prokaryotic desaturase, wherein desaturase activity of the recombinant prokaryotic desaturase is at least 1.5 fold higher than that of a corresponding native prokaryotic desaturase. In some aspects, the recombinant prokaryotic desaturase is a genetically engineered mutant form of a cyanobacterium desaturase. Exemplary cyanobacterium desaturases include glycerolipid Δ9-desaturase (DSG) from Synechococcus elongatus (Anacystis nidulans, PCC 6301). An amino acid sequence of the recombinant prokaryotic desaturase may include a mutation at one or more of the mutations listed in Table 2 below, with exemplary mutation including Arg or Lys at one or both of positions 69 and 240. In yet other aspects, i) the nucleic acid molecule is codon optimized for expression in a eukaryotic host; and/or ii) AT/CG ratios of the nucleic acid molecule are modified for expression in a eukaryotic host.

Additional aspects of the invention provide a transgenic eukaryotic host which is genetically engineered to contain and express a nucleic acid molecule that encodes a recombinant prokaryotic desaturase, wherein desaturase activity of the recombinant prokaryotic desaturase is at least 1.5 fold higher than that of a corresponding native prokaryotic desaturase. In further aspects, the recombinant prokaryotic desaturase is a genetically engineered mutant form of a cyanobacterium desaturase, for example, glycerolipid Δ9-desaturase (DSG) from Synechococcus elongatus (Anacystis nidulans, PCC 6301). An amino acid sequence of the recombinant prokaryotic desaturase may include a mutation at one or more of the mutations listed in Table 2 or Table 3 below, with exemplary mutations including Arg or Lys at one or both of positions 69 and 240. The nucleic acid molecule may be codon optimized for expression in a eukaryotic host. In certain aspects, the transgenic eukaryotic host is a plant or plant cell; an animal or an animal cell; or a fungal cell. In additional aspects, when the transgenic eukaryotic host is a plant or plant cell; an animal or an animal cell; or a fungal cell, the recombinant prokaryotic desaturase may further comprise an endoplasmic reticulum retention sequence.

In some aspects, the transgenic eukaryotic host is a plant and the plant is an oil seed producing plant.

Further aspects of the invention provide products produced by or from a transgenic eukaryotic host which is genetically engineered to contain an express a nucleic acid molecule that encodes a recombinant prokaryotic desaturase, wherein desaturase activity of the recombinant prokaryotic desaturase is at least 1.5 fold higher than that of a corresponding native prokaryotic desaturase. Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

FIG. 1. Simplified illustration of 16C metabolism. 1, Elongation of 16:0 to 18:0 uses a distinct condensing enzyme, ketoacyl-ACP synthase II (KASII); 2, acyl-ACP thioesterase FatB, 16:0-ACP reaction, including export from the plastid; 3, 16:0-ACP desaturase desaturates 16:0 immediately upon its synthesis in the plastid as an ACP thioester; 4, acyl-ACP thioesterase FatA, thioesterase for unsaturated fatty acids (prefers 18:1); 5, desaturation of 16:0-CoA to 16:1 CoA, for example fat-5 from Caenorhabditis elegans; 6, desaturation of 16:0-glycerolipid molecules, for example DSG from Synechococcus elongatus (Anacystis nidulans, PCC 6301).

FIG. 2. Comparative depiction of nucleotide sequences of DSG (SEQ ID NO: 2) and Des9 (SEQ ID NO: 4). Length: 855, identical sites: 654 (78.4%), pairwise % identity: 78.4%, ungapped lengths of 2 sequences: mean: 844.5, std dev: 10.5, minimum: 834, maximum: 855. HRR: Histidine Rich Repeat.

FIG. 3. SignalP prediction of DSG ER targeting potential. DSG has an N-terminal sequence similar to those of eukaryotic integral membrane proteins.

FIGS. 4A and B. Fatty acid profiles of DES9 and wild type yeast. A. Fatty acid compositions of wild type and DES9-expressing yeast after 2 days growth in liquid culture. B. Total saturated and unsaturated fatty acids and conversion efficiency of 16:0 and 18:0 in DES9 and wild type. The 16:0 and 18:0 desaturation are reported as conversion of 16:0 to 16:1 and 18:0 to 18:1, respectively. Wild type yeast are open bars, DES9 are black bars.

FIG. 5. Percent conversion of 16:0 for parental DES9 and 22 mutants selected from first round random mutagenesis. Mutation sites indicated on the x-axis. Conversion is calculated as 16:0/(16:0+16:1)×100. To distinguish mutant DES9 derivatives with increased desaturase activity, we refer to them as DES9*. The DES9*Q240R value is one representative of 7 independent clones.

FIGS. 6A and B. Fatty acid profiles (A) and composition (B) for DES9, DES9*Q240R, wild type, and OLE1::ole1 in yeast.

FIG. 7. Percent conversion of 16:0 for parental DES9 and DES9* mutants. Seven selected DES9* mutants from a second round of random mutagenesis for which Q240 and the surrounding 6 amino acids were not mutagenized. E69K is one representative of two independent clones. Yeast cultures were grown for 2 d in SD-Ura medium.

FIGS. 8A-E. 16:0 levels of T2 bulk seeds in transgenic Arabidopsis plants expressing the recombinant cyanobacterial DSG gene with an ER retention signal (A); recombinant plant codon-optimized DES9 gene including an ER retention signal (B); recombinant cyanobacterial DSG gene wherein Q at position 240 has been substituted for R (C); recombinant plant codon-optimized DES9 gene wherein Q at position 240 has been substituted for R (D); recombinant plant codon-optimized DES9 gene wherein Q at position 240 has been substituted for R and E at position 69 has been exchanged for R (E). The proportion of total fatty acids that was 16:0 () or 16:1(O) varies from equivalent to wild type (on the right) to highly modified (on the left). Each pair of points results from analysis of an independent line expressing the indicated construct.

FIG. 9. Amino acids substitutions which increase 16:0 conversion. Selected DES9* which increase conversion of 16:0 to greater than 50%. Proposed topology model of the DES9 desaturase by TMHMM Server v. 2.0. The positions of selected random mutations are indicated by black circles. See Table 1 and Table 2 for residue substitutions.

FIG. 10. The levels of 16:0 in the seed of DES9, DSG-KSKIN and DES9*Q240R T3 homozygous Arabidopsis lines. The horizontal bars represent the average of T3 homozygous seeds from individual T2 plants, ±SD. Each black dot represents 16:0 level of T3 seeds from a individual T2 plant.

FIGS. 11A and B. A, amino acid sequence of native cyanobacterial glycerolipid Δ9-desaturase (DSG, SEQ ID NO: 1); B, nucleotide sequence that encodes the native enzyme (SEQ ID NO: 2).

FIGS. 12A and B. A, amino acid sequence of codon optimized DSG (SEQ ID NO: 3) with a C-terminal GKSKIN endoplasmic reticulum retention sequence (underlined; SEQ ID NO: 5); and B, exemplary nucleotide sequence (SEQ ID NO: 4) that encodes the codon optimized DSG, which also includes a 3′ “TGA” stop codon.

FIG. 13 A-BB. Exemplary amino acid sequences of cyanobacterial desaturases listed in Table 1.

FIG. 14. Levels of 16:0 in T2 red seeds of individual lines of DES9*Q240R-transformed Camelina. Data represent the levels of 16:0 from analysis of 6 randomly chosen red transgenic Camelina seed from each T1 line.

FIG. 15. Levels of 16:0 in T2 red seeds of individual lines of DES9*26-transformed Camelina. Data represent the levels of 16:0 from analysis of 6 randomly chosen red transgenic Camelina seed from each T1 line.

### DETAILED DESCRIPTION

The invention provides recombinant desaturase enzymes that, when expressed in heterologous eukaryotic host cells, decrease the amounts of saturated fatty acids in the host cells, and/or increase the amounts of unsaturated fatty acids in the host cells, or both. Various aspects of the invention include but are not limited to: the modified (recombinant, mutant, etc.) desaturase enzymes described herein; methods of obtaining the recombinant enzymes; transgenic cells and organisms that contain active forms of the recombinant enzymes; methods of making the transgenic cells and/or organisms; and products made by or from the transgenic cells and/or organisms.

The following definitions are used throughout:

The terms “protein”, “polypeptide” and “peptide” refer to contiguous chains of amino acids that are covalently bonded (linked) to each other by peptide (amide) bonds. In general, a peptide contains up to about 50 amino acids and a polypeptide contains about 50 or more amino acids. Proteins may contain one or more than one polypeptide. Those of skill in the art will recognize that these definitions are considered somewhat arbitrary, and these terms may be used interchangeably herein. The terms encompass amino acid polymers that are synthesized (transcribed and translated) in vivo and amino acid polymers that are chemically synthesized using procedures well known to those skilled in the art.

As used herein, the terms “nucleic acid” or “polynucleotide” or “nucleic acid molecule” refer to deoxyribonucleotides or ribonucleotides and polymers thereof in either single- or double-stranded form. Exemplary nucleic acids include DNA (including cDNA), RNA (e.g. mRNA, tRNA, rRNA, etc.), and hybrids thereof.

The term “gene” means a segment of DNA that encodes a biologically active RNA, which may be further translated into a polypeptide chain. The term may or may not include regions preceding and following the coding region as well as intervening sequences (introns) between individual coding segments (exons). As used herein, a gene may be a recombinant or genetically engineered DNA sequence that encodes a polypeptide of interest from which introns have been eliminated.

The terms “similarity”, “identity” and “homology” are known in the art. Generally, “identity” refers to a sequence comparison based on identical matches between corresponding identical positions in the sequence being compared. “Similarity” refers to a comparison between amino acid sequences, and take into account not only identical amino acids in corresponding positions, but also functionally similar amino acids in corresponding positions. Strictly speaking, “homology” between protein or DNA sequences is defined in terms of shared ancestry. However, those of skill in the art will recognize that these three terms are frequently used interchangeably and that convention is adopted herein. Percentages of “similarity”, “identity” and “homology” between or among sequences may be determined by various tools that are readily available to those of skill in the art. For example, see issued U.S. Pat. No. 8,507,650 (Gabriel, et al.) and references cited therein, the complete contents of which is hereby incorporated by referenced in entirety.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. Such nucleic acid could also be inserted into the genome of a host cell and still be considered isolated in that such host cell is not part of the natural environment for the nucleic acid or polypeptide.

As used herein, the term “ER retention signal” refers to an amino acid sequence (the ER retention signal peptide) attached to a polypeptide which causes the polypeptide to be retained and accumulated in the endoplasmic reticulum (ER).

As used herein, the term “heterologous” refers to e.g. polypeptide, nucleic acid, promoter, etc. that originates from a source foreign to a particular host cell. The particular host cell in which the heterologous (non-native) polypeptide, nucleic acid, etc. is expressed may be referred to as a “heterologous” host cell. The term “heterologous” may also be used to refer to a genetic element which does not occur in nature as being operably linked to other genetic elements. For example, a promoter may be referred to a as being heterologous to a operably linked coding region when that promoter and coding region are not occurring as being operably linked in nature.

As used herein, a DNA segment is referred to as “operably linked” when it is placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, DNA sequences that are operably linked are contiguous, and in the case of a signal sequence both contiguous and in reading phase. However, enhancers need not be contiguous with the coding sequences whose transcription they control. Linking is accomplished by ligation at convenient restriction sites or at adapters or linkers inserted in lieu thereof.

As used herein, the terms “plant” and “plant tissue” refer to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath. Plants also include vegetables and fruit plants. “Lower plants” is a collective term for three main groups of plants (mosses, liverworts and lichens) which do not have roots and produce spores to reproduce, rather than flowers. “Higher plants” refers to plants that have vascular tissue (as known as tracheophytes). “Seed producing plants” is a term referring to those plants that produce seed (Spermatophytes) and includes “Flowering plants”, which refers to seed-producing plants, also known as Angiospermae or Magnoliophyta, as well as the Gymnospermae. Plants may be grown (e.g. in a field or a greenhouse) for production of food, fuel or fiber or other uses (e.g. wood, ornamentals). All such plants are encompassed by the present invention.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell. The term “transformant” refers to a cell, tissue or organism that has undergone transformation.

As used herein, the term “transgenic” refers to cells, cell cultures, organisms (e.g., plants), and progeny which comprise a modified or foreign (heterologous) gene, wherein the modified or foreign gene is not originally present in the host organism. Transgenic organisms may receive the foreign gene by one of the various methods of transformation, but may also receive the transgene via conventional breeding techniques whereby at least one of the parent organisms comprises such a transgene.

“Recombinant” refers to a product of genetic engineering, e.g. a nucleic acid such as recombinant DNA, a protein that results from the expression of recombinant DNA, and recombinant cells or organisms that are transformed with recombinant DNA.

### Recombinant Proteins

In some aspects, the invention provides mutant recombinant desaturase proteins (enzymes) with enhanced desaturase activity. A fatty acid desaturase is an enzyme that removes two hydrogen atoms from a fatty acid, thereby introducing or creating a carbon/carbon double bond in the backbone of the fatty acid and “desaturating” the fatty acid. The mutant enzymes are recombinant enzymes that have been purposefully modified and selected to have enhanced activity i.e. the enzymes do not occur in nature. Generally, the protein that is modified is prokaryotic and the heterologous host cell is eukaryotic. The enhanced activity of the desaturase is maintained when the desaturase is expressed within the eukaryotic host.

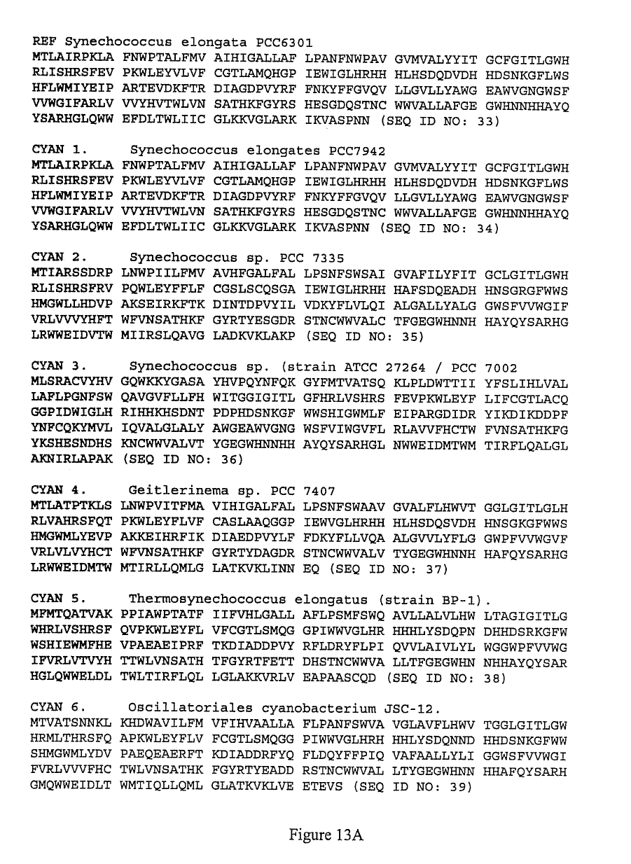

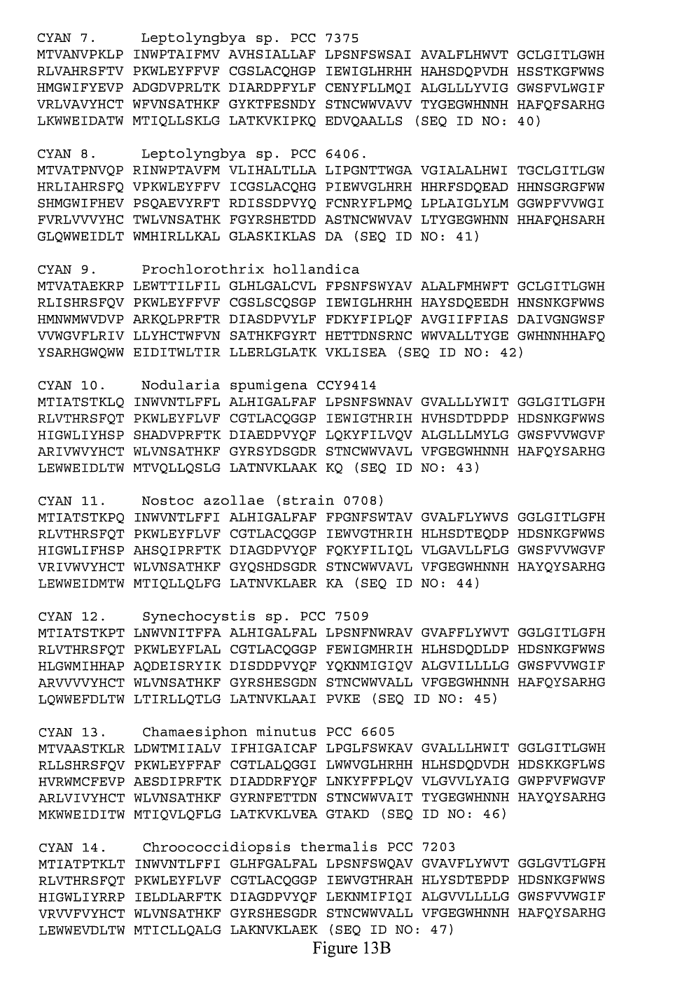

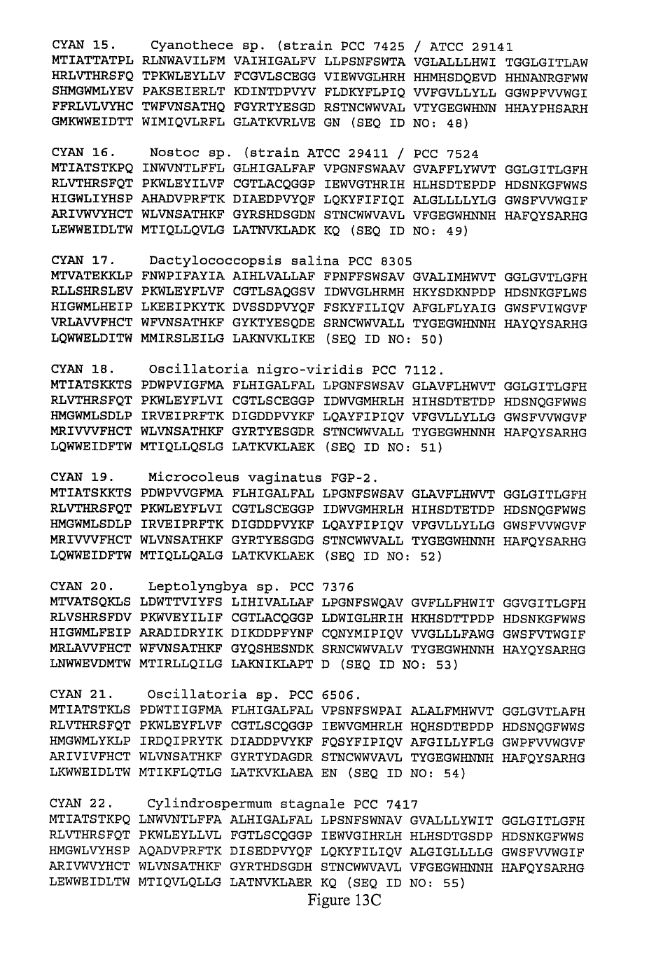

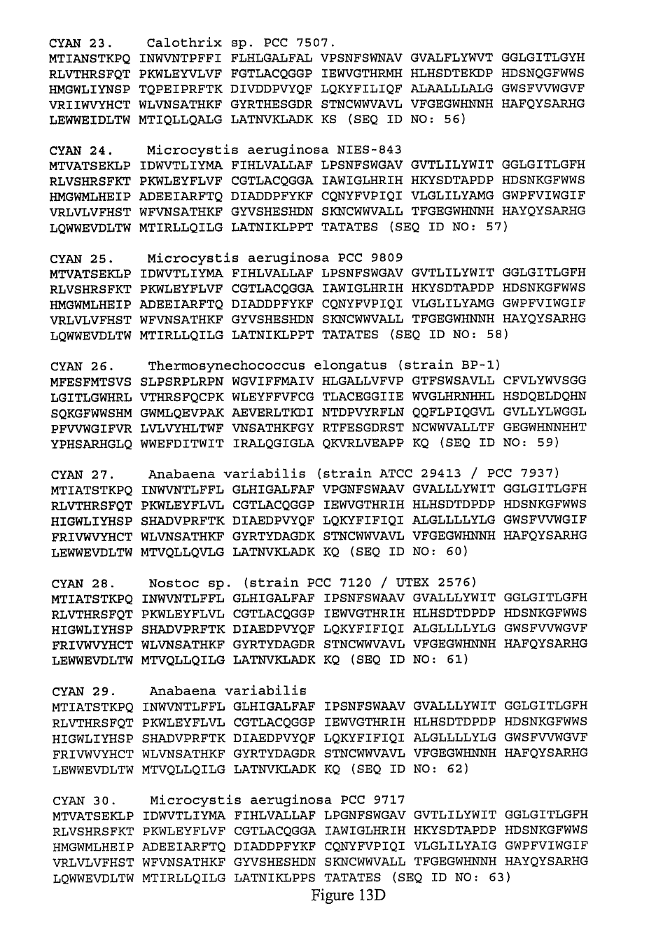

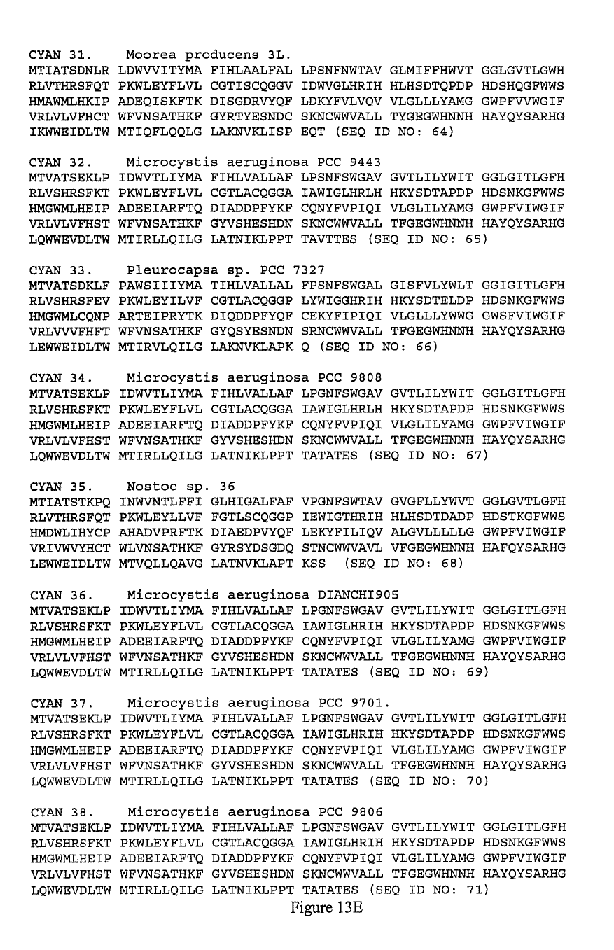

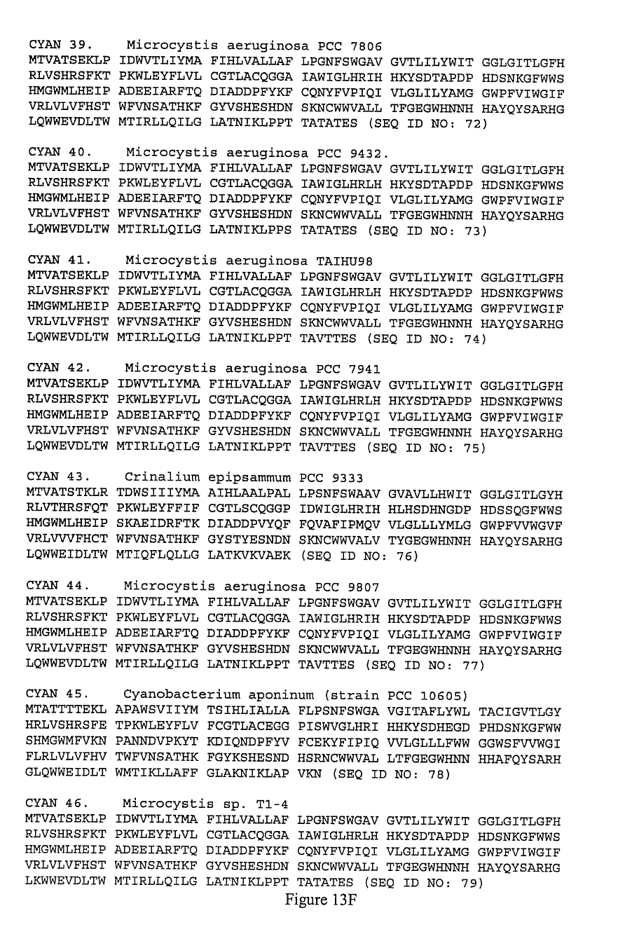

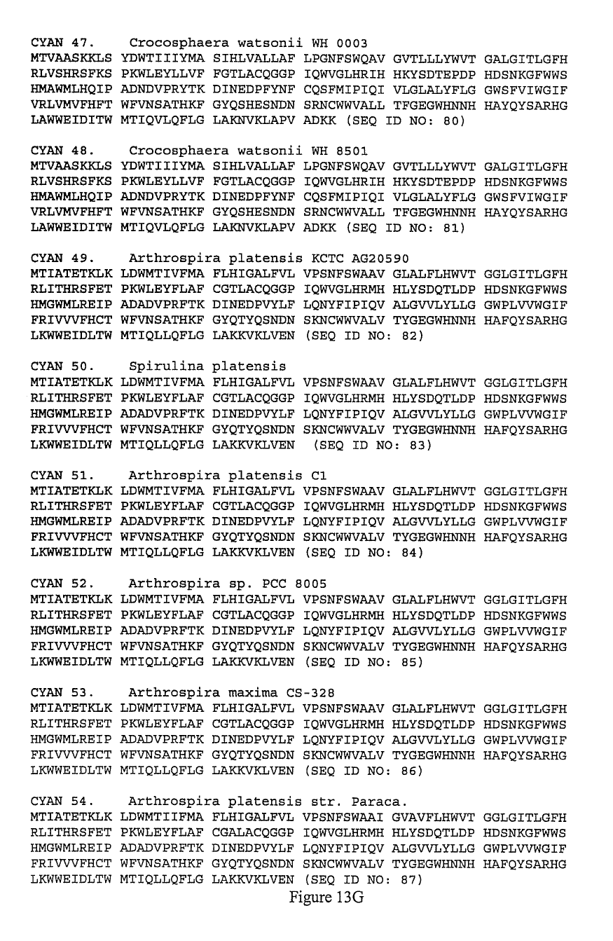

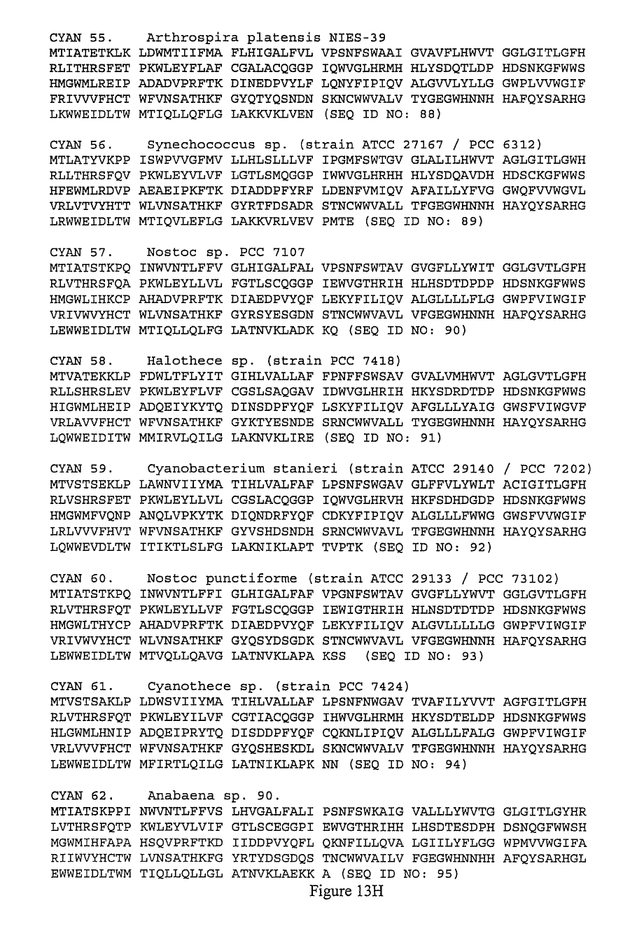

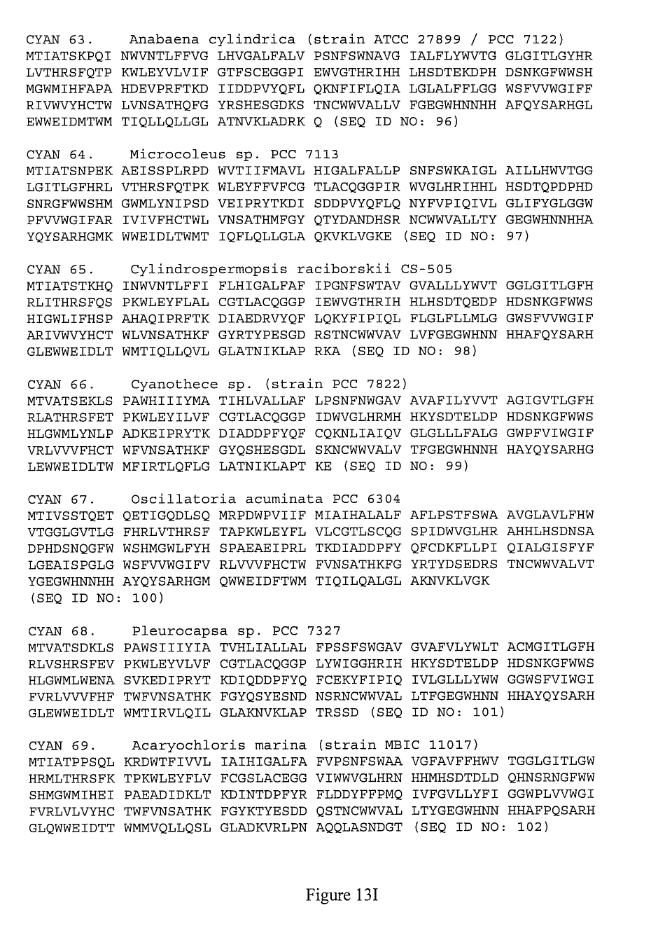

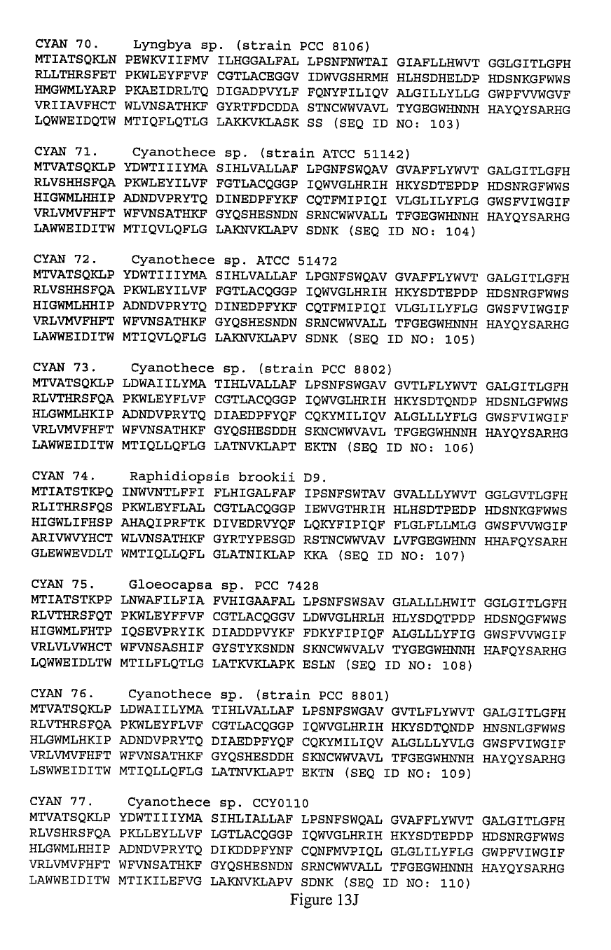

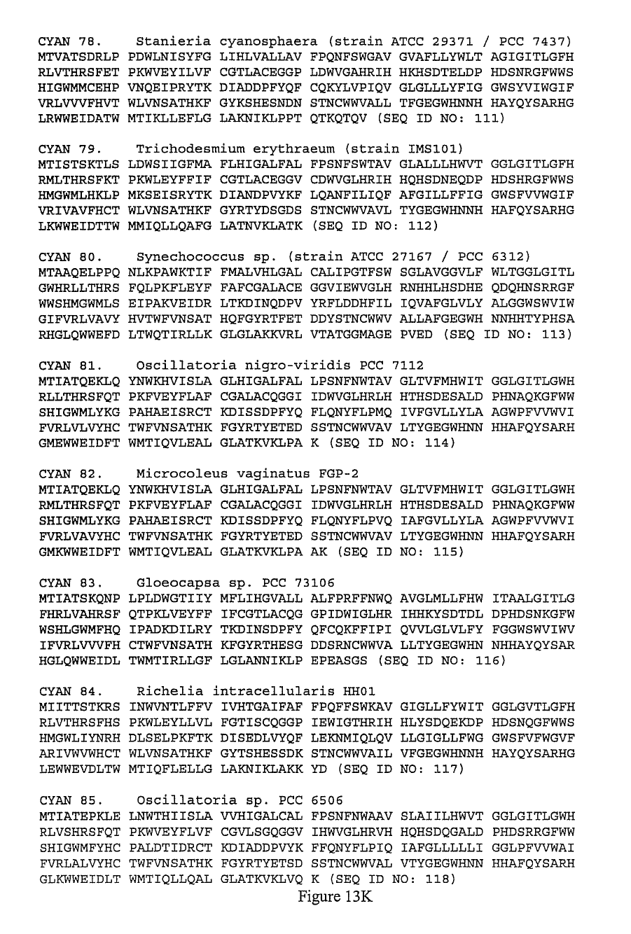

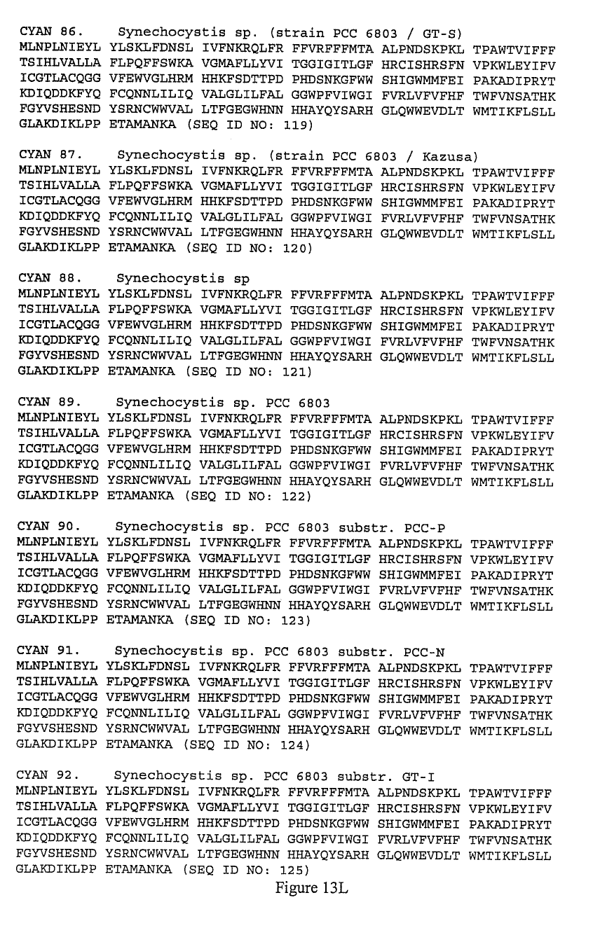

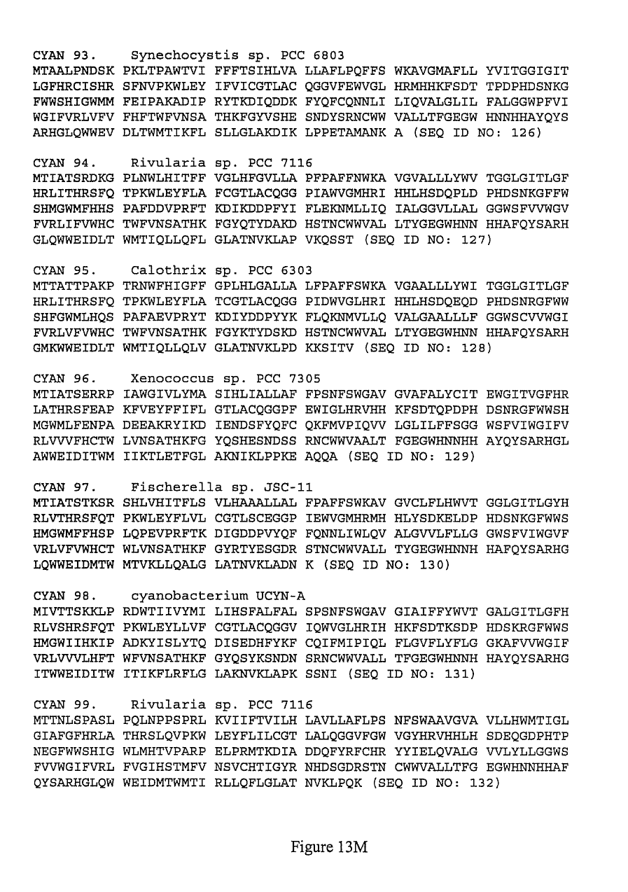

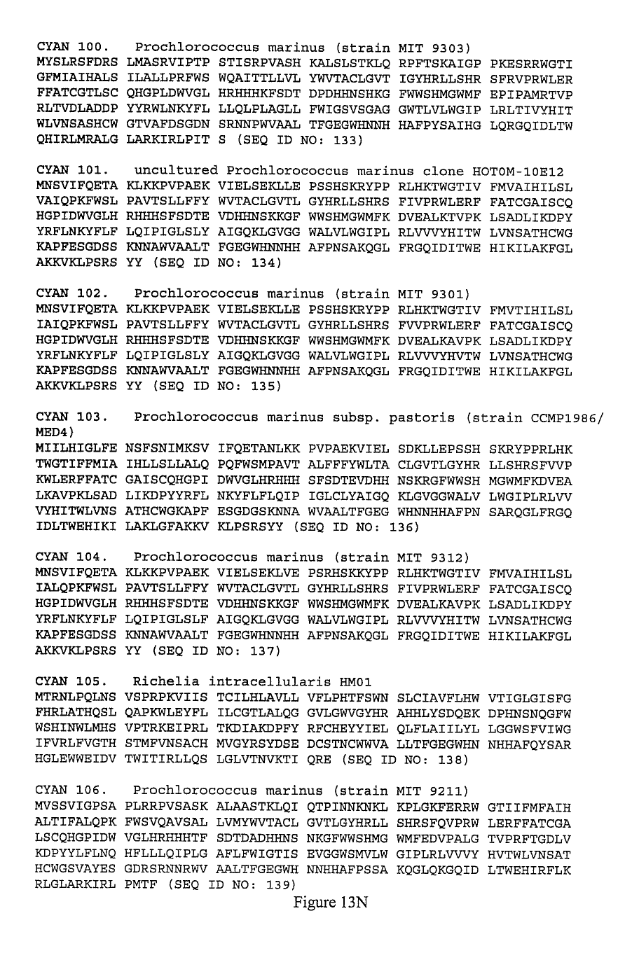

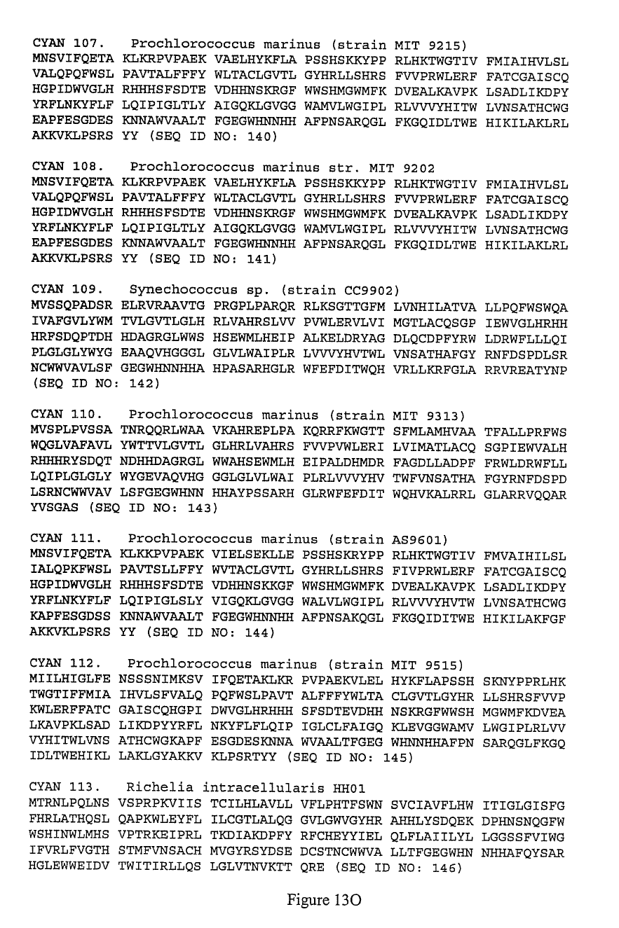

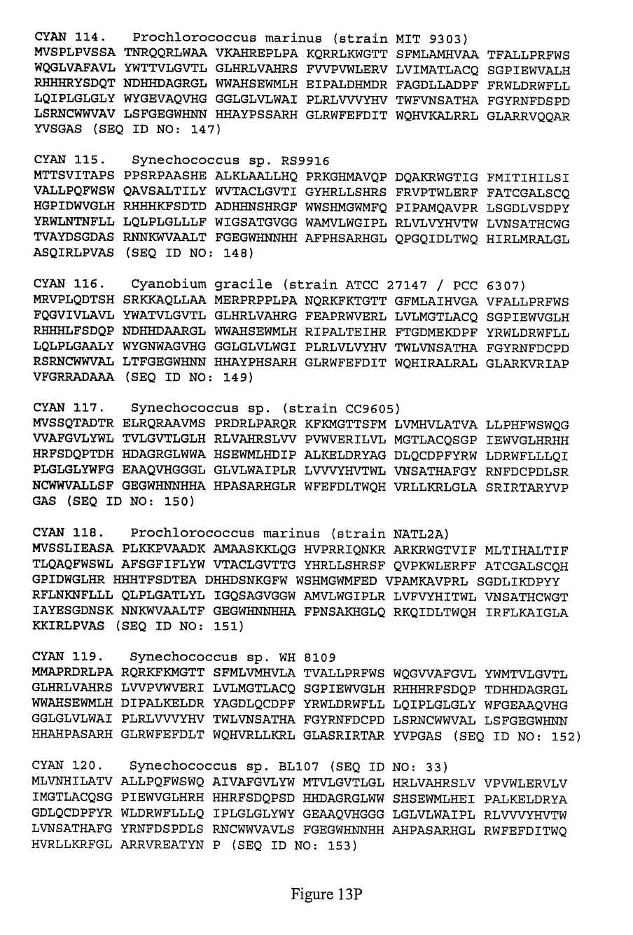

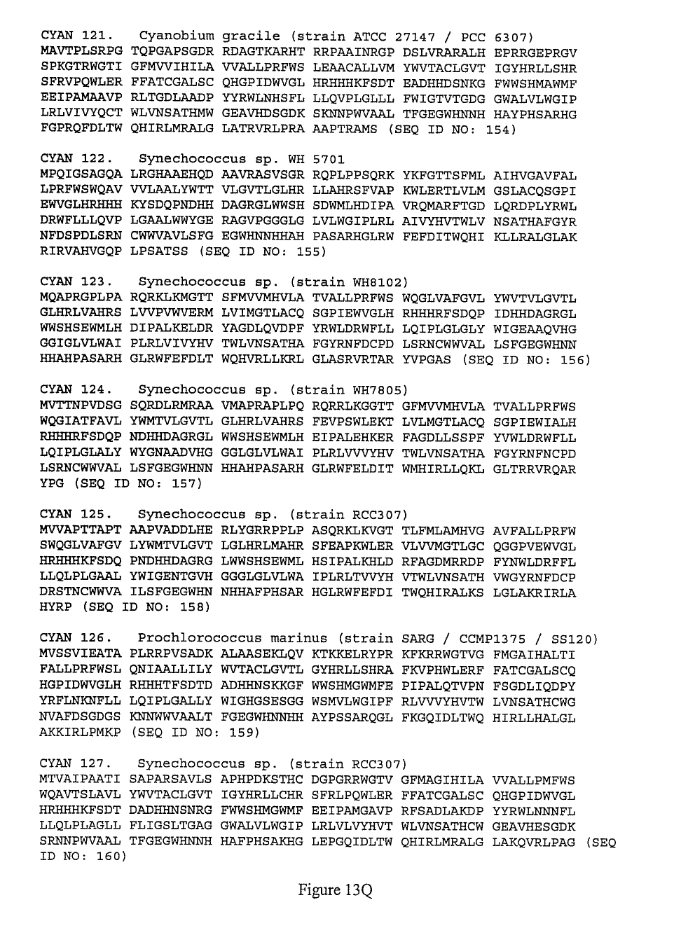

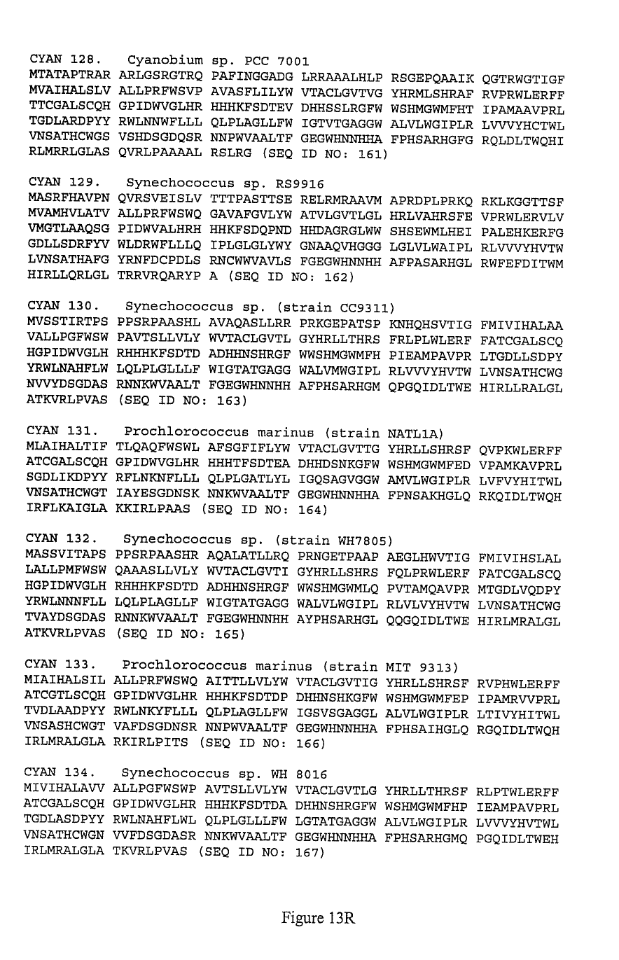

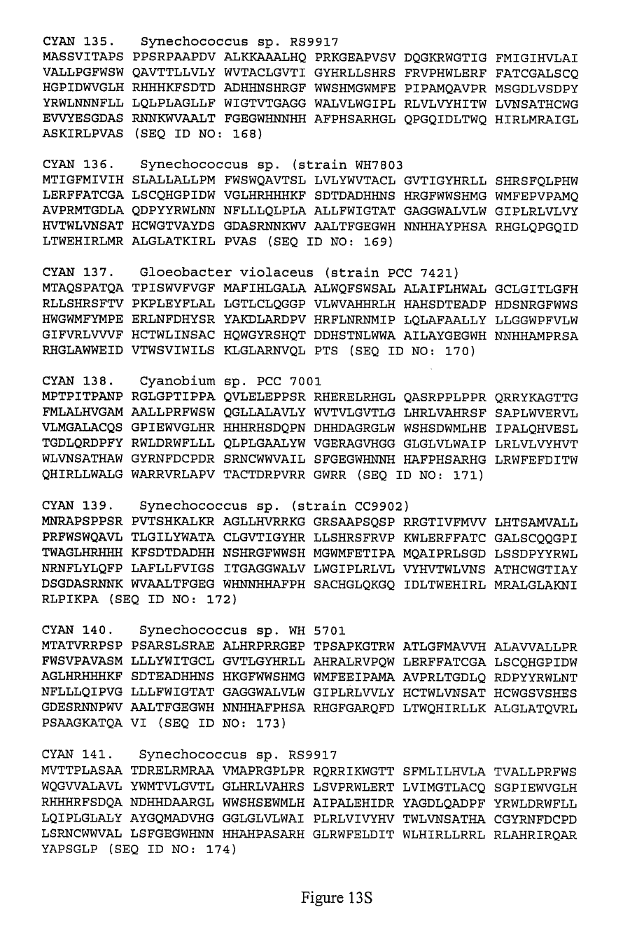

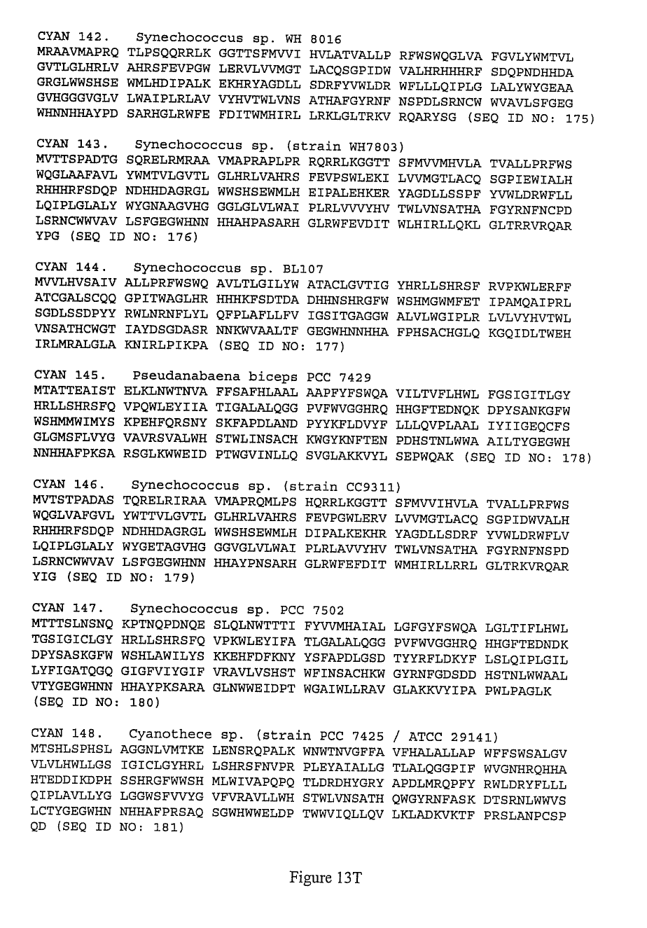

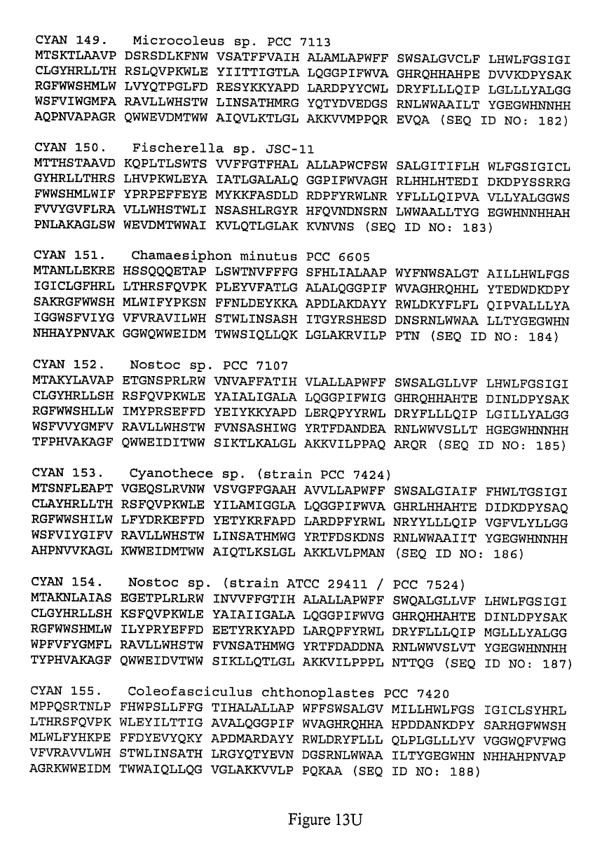

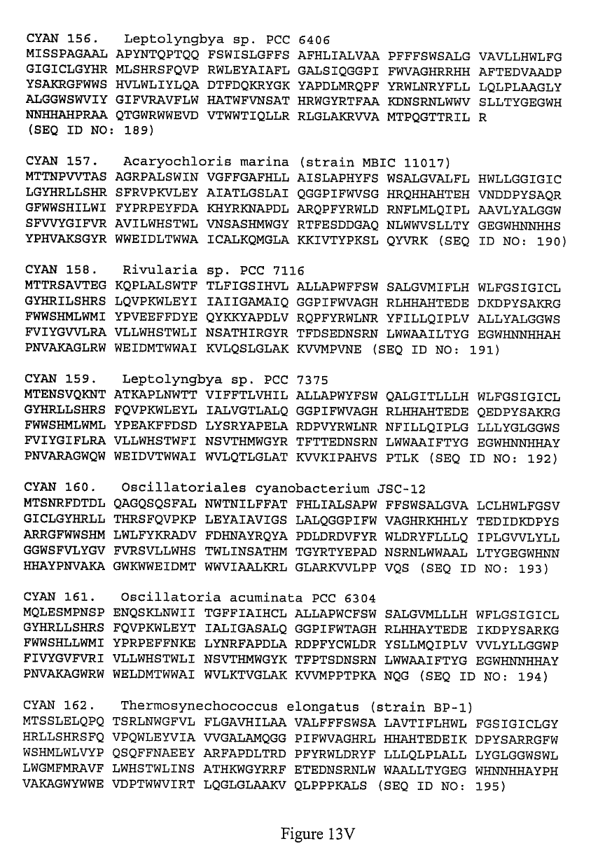

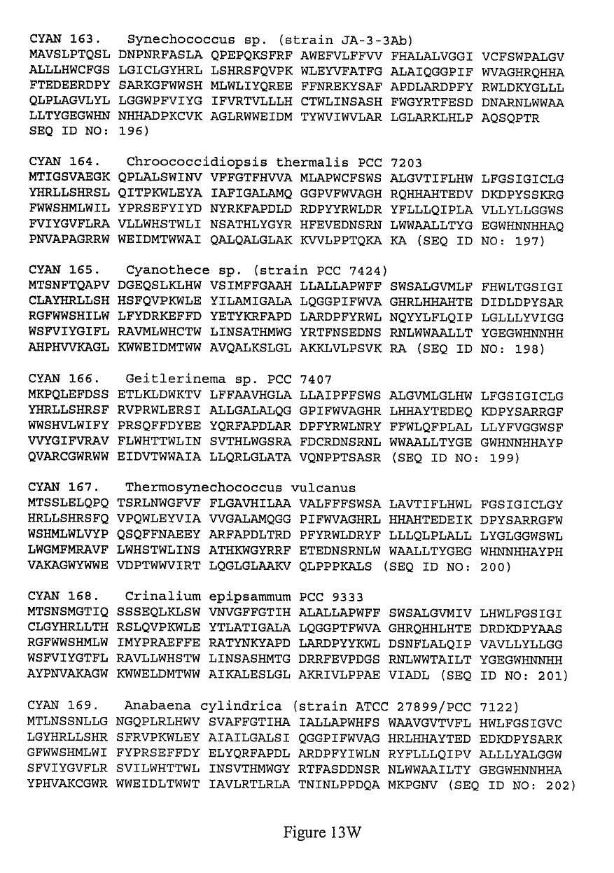

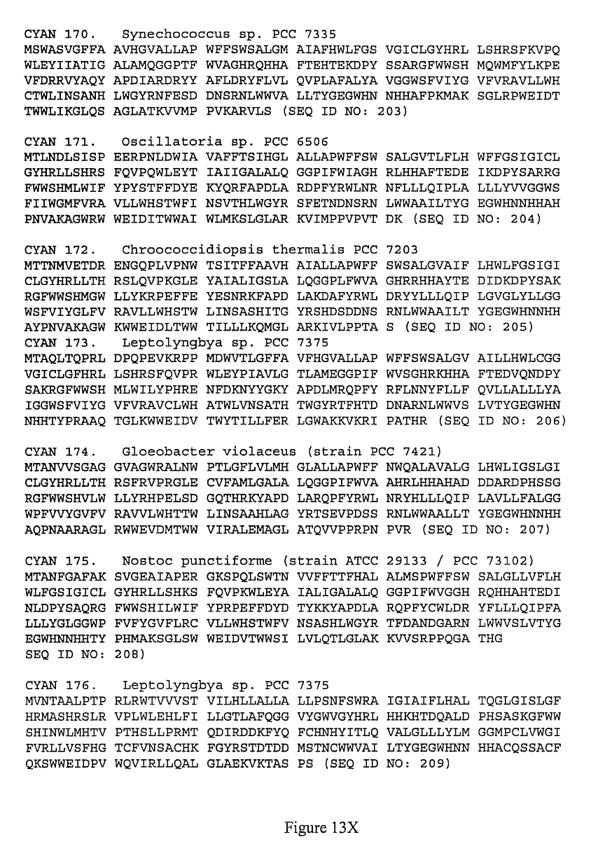

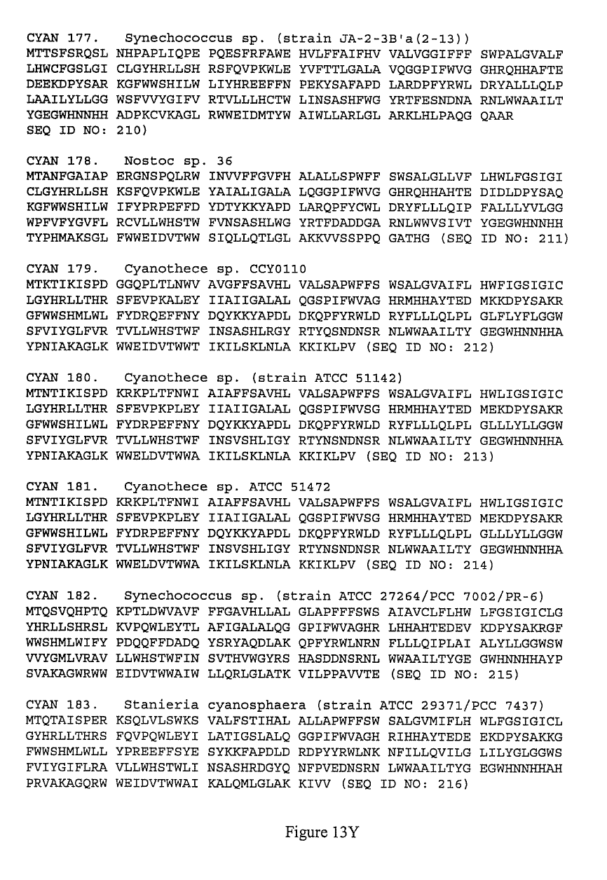

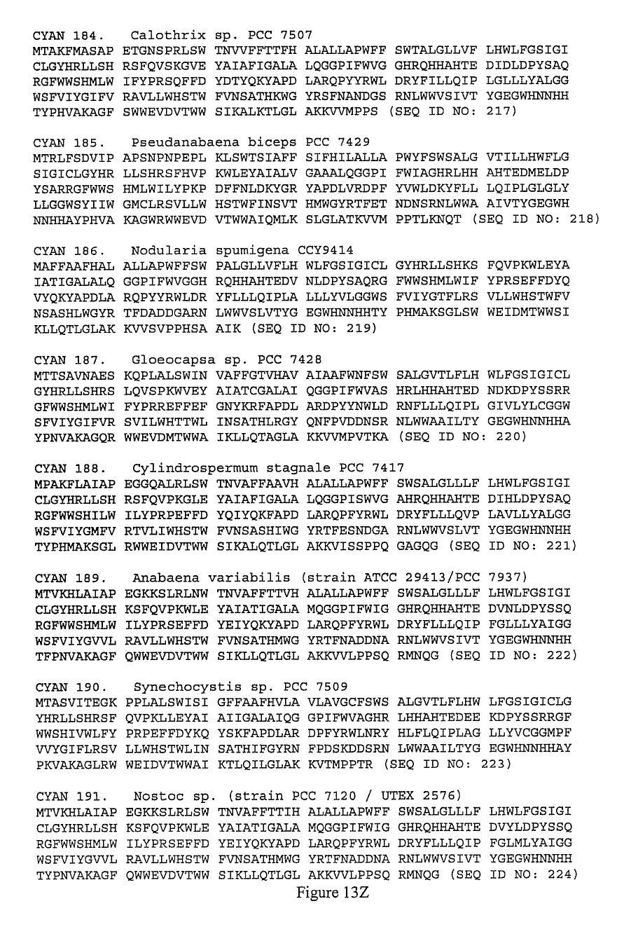

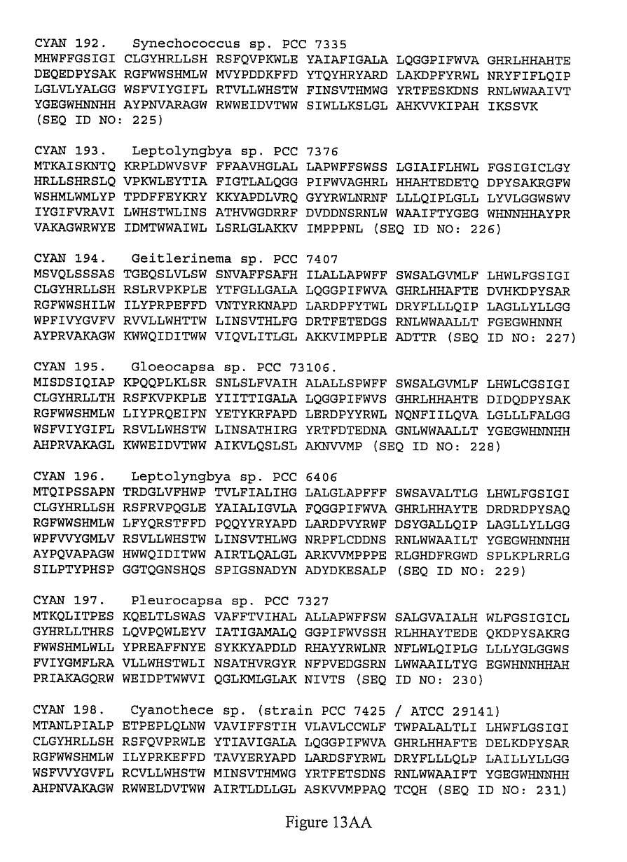

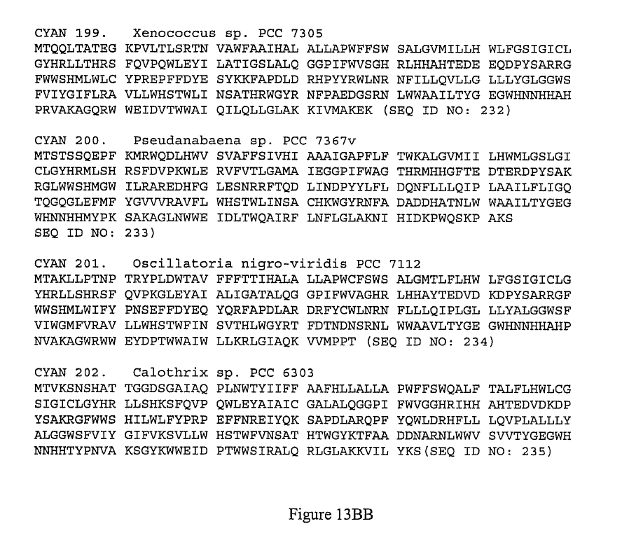

Desaturases are classified as “delta”, indicating that the double bond is created at a fixed position from the carboxyl group of a fatty acid (for example, Δ9 desaturase creates a double bond at the 9th position from the carboxyl end); and “omega” (e.g. ω3desaturase), indicating the double bond is created between the third and fourth carbon from the methyl end of the fatty acid. Exemplary categories of desaturases that may be used in the practice of the invention include but are not limited to cyanobacterial desaturases, including 49 desaturases, etc., as listed in Table 1 Amino acid sequences of the desaturases are shown in FIG. 13A-BB.

TABLE 1
Exemplary cyanobacterial desaturases

| | UniProt | | |
|---|---|---|---|
| | Reference | % Identity | Species |
| CYAN 1 | Q31K28 | 100 | Synechococcus elongatus (strain PCC 7942) (Anacystis |
| | | | nidulans R2) |
| CYAN 2 | B4WGN6 | 64 | Synechococcus sp PCC 7335 |
| CYAN 3 | B1XIS7 | 64 | Synechococcus sp (strain ATCC 27264/PCC 7002/PR-6) |
| | | | (Agmenellumquadruplicatum) |
| CYAN 4 | K9S2V5 | 63 | Geitlerinema sp PCC 7407 |
| CYAN 5 | Q8DGD8 | 64 | Thermosynechococcus elongatus (strain BP-1) |
| CYAN 6 | K8GL66 | 63 | Oscillatoriales cyanobacterium JSC-12 |
| CYAN 7 | K9FLM6 | 62 | Leptolyngbya sp PCC 7375 |
| CYAN 8 | L8L355 | 64 | Leptolyngbya sp PCC 6406 |
| CYAN 9 | Q6YPB5 | 62 | Prochlorothrix hollandica |
| CYAN 10 | A0ZMU4 | 63 | Nodularia spumigena CCY9414. |
| CYAN 11 | D7E3I2 | 62 | Nostoc azollae (strain 0708) (Anabaena azollae (strain 0708)) |
| CYAN 12 | L8KXS9 | 64 | Synechocystis sp PCC 7509 |
| CYAN 13 | K9UNL1 | 61 | Chamaesiphon minutus PCC 6605 |
| CYAN 14 | K9U4I0 | 62 | Chroococcidiopsis thermalis PCC 7203 |
| CYAN 15 | B8HMA9 | 60 | Cyanothece sp (strain PCC 7425/ATCC 29141) |
| CYAN 16 | K9QYI4 | 62 | Nostoc sp. (strain ATCC 29411/PCC 7524). |
| CYAN 17 | K9YSU6 | 60 | Dactylococcopsis salina PCC 8305 |
| CYAN 18 | K9VLQ4 | 60 | Oscillatoria nigro-viridis PCC 7112. |
| CYAN 19 | F5UI80 | 60 | Microcoleus vaginatus FGP-2 |
| CYAN 20 | K9Q0S8 | 60 | Leptolyngbya sp PCC 7376 |
| CYAN 21 | D8G8W5 | 57 | Oscillatoria sp PCC 6506 |
| CYAN 22 | K9WZC6 | 62 | Cylindrospermum stagnale PCC 7417 |
| CYAN 23 | K9PJ93 | 60 | Calothrix sp PCC 7507 |
| CYAN 24 | B0JWW8 | 64 | Microcystis aeruginosa (strain NIES-843) |
| CYAN 25 | I4HJ49 | 64 | Microcystis aeruginosa PCC 9809. |
| CYAN 26 | Q8DID7 | 60 | Thermosynechococcus elongatus (strain BP-1) |
| CYAN 27 | Q3M5C5 | 61 | Anabaena variabilis (strain ATCC 29413/PCC 7937). |
| CYAN 28 | Q44502 | 60 | Nostoc sp (strain PCC 7120/UTEX 2576) |
| CYAN 29 | Q79F73 | 60 | Anabaena variabilis. |
| CYAN 30 | I4FW83 | 64 | Microcystis aeruginosa PCC 9717. |
| CYAN 31 | F4XTT0 | 58 | Moorea producens 3L |
| CYAN 32 | I4G6K1 | 63 | Microcystis aeruginosa PCC 9443. |
| CYAN 33 | K9T0H7 | 61 | Pleurocapsa sp. PCC 7327. |
| CYAN 34 | I4HL03 | 63 | Microcystis aeruginosa PCC 9808. |
| CYAN 35 | Q704F2 | 60 | Nostoc sp. 36. |
| CYAN 36 | L8NN03 | 63 | Microcystis aeruginosa DIANCHI905. |
| CYAN 37 | I4IKL6 | 63 | Microcystis aeruginosa PCC 9701. |
| CYAN 38 | I4GWT3 | 63 | Microcystis aeruginosa PCC 9806. |
| CYAN 39 | A8YEP3 | 63 | Microcystis aeruginosa PCC 7806. |
| CYAN 40 | I4FDA9 | 63 | Microcystis aeruginosa PCC 9432. |
| CYAN 41 | L7EDK5 | 63 | Microcystis aeruginosa TAIHU98. |
| CYAN 42 | I4GP43 | 63 | Microcystis aeruginosa PCC 7941. |
| CYAN 43 | K9VUR7 | 61 | Crinalium epipsammum PCC 9333 |
| CYAN 44 | I4HCE5 | 63 | Microcystis aeruginosa PCC 9807. |
| CYAN 45 | K9Z0U8 | 62 | Cyanobacterium aponinum (strain PCC 10605) |
| CYAN 46 | I4IJI7 | 63 | Microcystis sp. T1-4. |
| CYAN 47 | G5JBB4 | 60 | Crocosphaera watsonii WH 0003 |
| CYAN 48 | Q4BXX0 | 60 | Crocosphaera watsonii WH 8501. |
| CYAN 49 | B2M1X7 | 59 | Arthrospira platensis KCTC AG20590 |
| CYAN 50 | O33722 | 59 | Spirulina platensis. |
| CYAN 51 | K1X365 | 59 | Arthrospira platensis C1. |
| CYAN 52 | H1WCT5 | 59 | Arthrospira sp. PCC 8005. |
| CYAN 53 | B5W728 | 59 | Arthrospira maxima CS-328. |
| CYAN 54 | K6E4V5 | 59 | Arthrospira platensis str. Paraca. |
| CYAN 55 | D4ZYK1 | 59 | Arthrospira platensis NIES-39. |
| CYAN 56 | K9RTW5 | 61 | Synechococcus sp (strain ATCC 27167/PCC 6312) |
| CYAN 57 | K9Q6J3 | 60 | Nostoc sp. PCC 7107. |
| CYAN 58 | K9YBE6 | 59 | Halothece sp (strain PCC 7418) (Synechococcus sp (strain |
| | | | PCC 7418)) |
| CYAN 59 | K9YJB2 | 61 | Cyanobacterium stanieri (strain ATCC 29140/PCC 7202) |
| CYAN 60 | B2IW55 | 59 | Nostoc punctiforme (strain ATCC 29133/PCC 73102) |
| CYAN 61 | B7KE48 | 59 | Cyanothece sp (strain PCC 7424) (Synechococcus sp |
| | | | (strain ATCC29155)) |
| CYAN 62 | K7W6Q5 | 60 | Anabaena sp 90 |
| CYAN 63 | K9ZLP7 | 61 | Anabaena cylindrica (strain ATCC 27899/PCC 7122) |
| CYAN 64 | K9WDB9 | 58 | Microcoleus sp PCC 7113 |
| CYAN 65 | D4TCG3 | 62 | Cylindrospermopsis raciborskii CS-505 |
| CYAN 66 | E0UBA3 | 60 | Cyanothece sp. (strain PCC 7822). |
| CYAN 67 | K9TSW5 | 59 | Oscillatoria acuminata PCC 6304 |
| CYAN 68 | K9SZC6 | 59 | Pleurocapsa sp PCC 7327 |
| CYAN 69 | B0C917 | 57 | Acaryochloris marina (strain MBIC 11017) |
| CYAN 70 | A0Z057 | 58 | Lyngbya sp (strain PCC 8106) (Lyngbya aestuarii (strain |
| | | | CCY9616)) |
| CYAN 71 | B1WWX9 | 58 | Cyanothece sp (strain ATCC 51142) |
| CYAN 72 | G6H0M8 | 58 | Cyanothece sp. ATCC 51472. |
| CYAN 73 | C7QWM4 | 60 | Cyanothece sp. (strain PCC 8802) (Synechococcus sp. |
| | | | (strain RF-2)). |
| CYAN 74 | D4TVH8 | 61 | Raphidiopsis brookii D9. |
| CYAN 75 | K9X7Q6 | 57 | Gloeocapsa sp PCC 7428 |
| CYAN 76 | B7JXH1 | 60 | Cyanothece sp (strain PCC 8801) (Synechococcus sp |
| | | | (strain PCC 8801/RF-1)) |
| CYAN 77 | A3IQ76 | 58 | Cyanothece sp. CCY0110. |
| CYAN 78 | K9XVB6 | 59 | Stanieria cyanosphaera (strain ATCC 29371/PCC 7437) |
| CYAN 79 | Q115U2 | 54 | Trichodesmium erythraeum (strain IMS101) |
| CYAN 80 | K9RQV6 | 58 | Synechococcus sp (strain ATCC 27167/PCC 6312) |
| CYAN 81 | K9VJM8 | 55 | Oscillatoria nigro-viridis PCC 7112. |
| CYAN 82 | F5UI79 | 55 | Microcoleus vaginatus FGP-2 |
| CYAN 83 | L8LIF8 | 57 | Gloeocapsa sp PCC 73106 |
| CYAN 84 | M1X4J8 | 57 | Richelia intracellularis HH01 |
| CYAN 85 | D8G8W6 | 57 | Oscillatoria sp PCC 6506 |
| CYAN 86 | F7UT40 | 60 | Synechocystis sp (strain PCC 6803/GT-S) |
| CYAN 87 | Q55406 | 60 | Synechocystis sp. (strain PCC 6803/Kazusa). |
| CYAN 88 | Q79F68 | 60 | Synechocystis sp. |
| CYAN 89 | M1MM94 | 60 | Synechocystis sp. PCC 6803. |
| CYAN 90 | H0PGV4 | 60 | Synechocystis sp. PCC 6803 substr. PCC-P. |
| CYAN 91 | H0PC87 | 60 | Synechocystis sp. PCC 6803 substr. PCC-N. |
| CYAN 92 | H0NZG9 | 60 | Synechocystis sp. PCC 6803 substr. GT-I. |
| CYAN 93 | L8API5 | 60 | Synechocystis sp. PCC 6803. |
| CYAN 94 | K9RHM8 | 59 | Rivularia sp PCC 7116 |
| CYAN 95 | K9UYF3 | 58 | Calothrix sp PCC 6303 |
| CYAN 96 | L8M138 | 58 | Xenococcus sp PCC 7305 |
| CYAN 97 | G6FW79 | 57 | Fischerella sp JSC-11 |
| CYAN 98 | D3EP80 | 54 | cyanobacterium UCYN-A |
| CYAN 99 | K9RL54 | 55 | Rivularia sp PCC 7116 |
| CYAN 100 | A2CDR5 | 52 | Prochlorococcus marinus (strain MIT 9303) |
| CYAN 101 | Q1PJN0 | 53 | uncultured Prochlorococcus marinus clone HOT0M-10E12. |
| CYAN 102 | A3PFG0 | 53 | Prochlorococcus marinus (strain MIT 9301). |
| CYAN 103 | Q7UZJ2 | 53 | Prochlorococcus marinus subsp. pastoris (strain |
| | | | CCMP1986/MED4). |
| CYAN 104 | Q317X0 | 52 | Prochlorococcus marinus (strain MIT 9312). |
| CYAN 105 | M1WMZ5 | 50 | Richelia intracellularis HM01 |
| CYAN 106 | A9BDB7 | 53 | Prochlorococcus marinus (strain MIT 9211) |
| CYAN 107 | A8G7H7 | 52 | Prochlorococcus marinus (strain MIT 9215). |
| CYAN 108 | B9NZ15 | 52 | Prochlorococcus marinus str. MIT 9202. |
| CYAN 109 | Q3AUL7 | 54 | Synechococcus sp (strain CC9902) |
| CYAN 110 | Q7V409 | 52 | Prochlorococcus marinus (strain MIT 9313). |
| CYAN 111 | A2BTQ3 | 52 | Prochlorococcus marinus (strain AS9601) |
| CYAN 112 | A2BZ58 | 52 | Prochlorococcus marinus (strain MIT 9515) |
| CYAN 113 | M1X519 | 50 | Richelia intracellularis HH01. |
| CYAN 114 | A2CDR3 | 52 | Prochlorococcus marinus (strain MIT 9303) |
| CYAN 115 | Q05VA8 | 50 | Synechococcus sp RS9916 |
| CYAN 116 | K9PA26 | 52 | Cyanobium gracile (strain ATCC 27147/PCC 6307) |
| CYAN 117 | Q3AGL3 | 53 | Synechococcus sp (strain CC9605) |
| CYAN 118 | Q46IB7 | 51 | Prochlorococcus marinus (strain NATL2A) |
| CYAN 119 | D0CMZ5 | 53 | Synechococcus sp. WH 8109. |
| CYAN 120 | Q061T5 | 54 | Synechococcus sp. BL107. |
| CYAN 121 | K9PAN7 | 51 | Cyanobium gracile (strain ATCC 27147/PCC 6307) |
| CYAN 122 | A3YXQ3 | 54 | Synechococcus sp WH 5701 |
| CYAN 123 | Q7U3Q3 | 53 | Synechococcus sp. (strain WH8102). |
| CYAN 124 | A4CX93 | 53 | Synechococcus sp. (strain WH7805). |
| CYAN 125 | A5GWN7 | 53 | Synechococcus sp (strain RCC307) |
| CYAN 126 | Q7V9K0 | 53 | Prochlorococcus marinus (strain SARG/CCMP1375/SS120) |
| CYAN 127 | A5GWN9 | 51 | Synechococcus sp (strain RCC307) |
| CYAN 128 | B5II65 | 51 | Cyanobium sp PCC 7001 |
| CYAN 129 | Q05VB0 | 51 | Synechococcus sp RS9916 |
| CYAN 130 | Q0I6E1 | 51 | Synechococcus sp (strain CC9311) |
| CYAN 131 | A2C5D8 | 51 | Prochlorococcus marinus (strain NATL1A). |
| CYAN 132 | A4CX91 | 50 | Synechococcus sp (strain WH7805) |
| CYAN 133 | Q7V407 | 52 | Prochlorococcus marinus (strain MIT 9313). |
| CYAN 134 | J4IQ78 | 52 | Synechococcus sp. WH 8016. |
| CYAN 135 | A3Z3I3 | 51 | Synechococcus sp. RS9917. |
| CYAN 136 | A5GPH8 | 53 | Synechococcus sp (strain WH7803) |
| CYAN 137 | Q7NL91 | 52 | Gloeobacter violaceus (strain PCC 7421) |
| CYAN 138 | B5II63 | 51 | Cyanobium sp PCC 7001 |
| CYAN 139 | Q3AUL6 | 50 | Synechococcus sp (strain CC9902) |
| CYAN 140 | A3YXN0 | 53 | Synechococcus sp WH 5701 |
| CYAN 141 | A3Z3I1 | 51 | Synechococcus sp RS9917 |
| CYAN 142 | J4IQE0 | 53 | Synechococcus sp. WH 8016. |
| CYAN 143 | A5GPH6 | 52 | Synechococcus sp (strain WH7803) |
| CYAN 144 | Q061T6 | 50 | Synechococcus sp. BL107. |
| CYAN 145 | L8N2H5 | 47 | Pseudanabaena biceps PCC 7429 |
| CYAN 146 | Q0I6E3 | 53 | Synechococcus sp (strain CC9311) |
| CYAN 147 | K9SYK7 | 47 | Synechococcus sp PCC 7502 |
| CYAN 148 | B8HKQ9 | 47 | Cyanothece sp (strain PCC 7425/ATCC 29141) |
| CYAN 149 | K9W807 | 48 | Microcoleus sp PCC 7113 |
| CYAN 150 | G6FT45 | 47 | Fischerella sp JSC-11 |
| CYAN 151 | K9UMP5 | 46 | Chamaesiphon minutus PCC 6605 |
| CYAN 152 | K9QG50 | 45 | Nostoc sp PCC 7107 |
| CYAN 153 | B7KMV9 | 45 | Cyanothece sp (strain PCC 7424) (Synechococcus sp |
| | | | (strain ATCC29155)) |
| CYAN 154 | K9QUE8 | 45 | Nostoc sp (strain ATCC 29411/PCC 7524) |
| CYAN 155 | B4VQZ8 | 46 | Coleofasciculus chthonoplastes PCC 7420 |
| CYAN 156 | L8LG99 | 44 | Leptolyngbya sp PCC 6406 |
| CYAN 157 | B0BYI0 | 46 | Acaryochloris marina (strain MBIC 11017) |
| CYAN 158 | K9RDP9 | 46 | Rivularia sp PCC 7116 |
| CYAN 159 | K9EQQ3 | 47 | Leptolyngbya sp PCC 7375 |
| CYAN 160 | K8GPD2 | 45 | Oscillatoriales cyanobacterium JSC-12 |
| CYAN 161 | K9TSH1 | 46 | Oscillatoria acuminata PCC 6304 |
| CYAN 162 | Q8DI71 | 44 | Thermosynechococcus elongatus (strain BP-1) |
| CYAN 163 | Q2JSA6 | 46 | Synechococcus sp (strain JA-3-3Ab) (Cyanobacteria |
| | | | bacteriumYellowstone A-Prime) |
| CYAN 164 | K9TZ86 | 46 | Chroococcidiopsis thermalis PCC 7203 |
| CYAN 165 | B7KIY1 | 47 | Cyanothece sp (strain PCC 7424) (Synechococcus sp |
| | | | (strain ATCC29155)) |
| CYAN 166 | K9S923 | 47 | Geitlerinema sp PCC 7407 |
| CYAN 167 | Q9ZAP7 | 44 | Thermosynechococcus vulcanus (Synechococcus |
| | | | vulcanus). |
| CYAN 168 | K9W3K6 | 45 | Crinalium epipsammum PCC 9333 |
| CYAN 169 | K9ZKI8 | 46 | Anabaena cylindrica (strain ATCC 27899/PCC 7122) |
| CYAN 170 | B4WNN0 | 46 | Synechococcus sp PCC 7335 |
| CYAN 171 | D8FTS6 | 45 | Oscillatoria sp PCC 6506 |
| CYAN 172 | K9TYS3 | 46 | Chroococcidiopsis thermalis PCC 7203 |
| CYAN 173 | K9EZU4 | 46 | Leptolyngbya sp PCC 7375 |
| CYAN 174 | Q7NCW0 | 47 | Gloeobacter violaceus (strain PCC 7421) |
| CYAN 175 | B2J6G0 | 43 | Nostoc punctiforme (strain ATCC 29133/PCC 73102) |
| CYAN 176 | K9ESA5 | 45 | Leptolyngbya sp PCC 7375 |
| CYAN 177 | Q2JN33 | 45 | Synechococcus sp (strain JA-2-3B a(2-13)) (Cyanobacteria |
| | | | bacteriumYellowstone B-Prime) |
| CYAN 178 | Q704E9 | 43 | Nostoc sp. 36. |
| CYAN 179 | A3IH72 | 43 | Cyanothece sp. CCY0110. |
| CYAN 180 | B1WTD9 | 44 | Cyanothece sp (strain ATCC 51142) |
| CYAN 181 | G6GRI4 | 44 | Cyanothece sp. ATCC 51472. |
| CYAN 182 | B1XHQ7 | 44 | Synechococcus sp (strain ATCC 27264/PCC 7002/PR-6) |
| | | | (Agmenellumquadruplicatum) |
| CYAN 183 | K9XY27 | 45 | Stanieria cyanosphaera (strain ATCC 29371/PCC 7437) |
| CYAN 184 | K9PRG7 | 46 | Calothrix sp PCC 7507 |
| CYAN 185 | L8MZ47 | 45 | Pseudanabaena biceps PCC 7429 |
| CYAN 186 | A0ZKJ6 | 45 | Nodularia spumigena CCY9414 |
| CYAN 187 | K9X8S7 | 45 | Gloeocapsa sp PCC 7428 |
| CYAN 188 | K9WX42 | 46 | Cylindrospermum stagnale PCC 7417 |
| CYAN 189 | Q3MAU1 | 45 | Anabaena variabilis (strain ATCC 29413/PCC 7937). |
| CYAN 190 | L8KSX1 | 46 | Synechocystis sp PCC 7509 |
| CYAN 191 | Q8YME3 | 45 | Nostoc sp (strain PCC 7120/UTEX 2576) |
| CYAN 192 | B4WUQ7 | 49 | Synechococcus sp PCC 7335 |
| CYAN 193 | K9PY01 | 43 | Leptolyngbya sp PCC 7376 |
| CYAN 194 | K9S8V1 | 45 | Geitlerinema sp PCC 7407 |
| CYAN 195 | L8LP31 | 44 | Gloeocapsa sp PCC 73106 |
| CYAN 196 | L8LB04 | 43 | Leptolyngbya sp PCC 6406 |
| CYAN 197 | K9T2Y0 | 43 | Pleurocapsa sp PCC 7327 |
| CYAN 198 | B8HM75 | 42 | Cyanothece sp (strain PCC 7425/ATCC 29141) |
| CYAN 199 | L8M4W5 | 45 | Xenococcus sp PCC 7305 |
| CYAN 200 | K9SK24 | 41 | Pseudanabaena sp PCC 7367 |
| CYAN 201 | K9VEM0 | 42 | Oscillatoria nigro-viridis PCC 7112 |
| CYAN 202 | K9V2Y2 | 44 | Calothrix sp PCC 6303 |

Exemplary prokaryotic sources of desaturase enzymes include but are not limited to: cyanobacteria, such as Stigonematales including species from the genera Capsosira, Chlorogloeopsis, Fischerella, Hapalosiphon, Mastigocladopsis, Mastigocladus, Nostochopsis, Stigonema, Symphyonema, Symphyonemopsis, Umezakia, Westiellopsis; Chroococcales including species from the genera Aphanocapsa, Aphanothece, Chamaesiphon, Chondrocystis, Chroococcus, Chroogloeocystis, Coelosphaerium, Crocosphaera, Cyanobacterium, Cyanobium, Cyanodictyon, Cyanosarcina, Cyanothece, Dactylococcopsis, Geminocystis, Gloeocapsa, Gloeothece, Euhalothece, Halothece, Johannesbaptistia, Merismopedia, Microcystis, Radiocystis, Rhabdoderma, Rubidibacter, Snowella, Sphaerocavum, Synechococcus, Synechocystis, Thermosynechococcus, Woronichinia; Gloeobacterales including species from the genus Gloeobacter; Nostocales including species from the genera Coleodesmium, Fremyella, Hassallia, Microchaete, Petalonema, Rexia, Spirirestis, Tolypothrix, Anabaena, Anabaenopsis, Aphanizomenon, Aulosira, Cyanospira, Cylindrospermopsis, Cylindrospermum, Mojavia, Nodularia, Nostoc, Raphidiopsis, Richelia, Trichormus, Calothrix, Gloeotrichia, Rivularia, Brasilonema, Scytonema, Scytonematopsis; Oscillatoriales including species from the genera Arthronema, Arthrospira, Blennothrix, Crinalium, Geitlerinema, Halomicronema, Halospirulina, Hydrocoleum, Jaaginema, Katagnymene, Komvophoron, Leptolyngbya, Limnothrix, Lyngbya, Microcoleus, Oscillatoria, Phormidium, Planktolyngbya, Planktothricoides, Planktothrix, Plectonema, Pseudanabaena, Pseudophormidium, Schizothrix, Spirulina, Starria, Symploca, Trichocoleus, Trichodesmium, Tychonema; Pleurocapsales including species from the genera Chroococcidiopsis, Dermocarpa, Dermocarpella, Myxosarcina, Pleurocapsa, Solentia, Stanieria, Xenococcus; Prochlorophytes including species from the genera Prochloron, Prochlorococcus, and Prochlorothrix.

Some aspects of the invention involve the use of prokaryotic desaturases from cyanobacteria, and an exemplary cyanobacterial enzyme that may be used in the practice of the invention is glycerolipid Δ9-desaturase (DSG) originally obtained from the cyanobacterium Synechococcus elongatus (Anacystis nidulans, PCC 6301), the amino acid sequence of which is shown in FIG. 11A (SEQ ID NO: 1). The nucleic acid sequence of the gene encoding this enzyme is shown in FIG. 11B (SEQ ID NO: 2). As a prokaryotic Δ9-desaturase, DSG uses ferredoxin as electron donor for desaturation, and introduces a cis-double bond at the Δ⁹ position of both 16- and 18-carbon saturated fatty acids linked to membrane lipids. According to aspects the invention, mutant DSG enzymes with enhanced desaturase activity have been developed by modifying the original, native (wild type) sequence.

The exemplary mutant DSGs of the invention each comprise at least one alteration/substitution in the primary amino acid sequence of the native sequence, and may contain a plurality of such changes. These substitutions, in combination with other modifications described herein, increase (augment, optimize, maximize, etc.) the desaturase activity of the DSG recombinant protein, and increased activity is maintained when the modified DSGs (DES9*) are expressed within eukaryotic host cells.

Any DSG residue substitution or combinations thereof that are identified by the methods described herein are encompassed by the present invention. Positions within the amino acid sequence which have been found to be of particular significance with respect to increasing desaturase activity, or in contributing to an increase in desaturase activity, are listed in Table 2 below. Exemplary amino acid substitutions for those positions are also listed. All possible combinations of these possible mutations, including mutations at a single (one) residue, and mutations at a plurality (more than one) of any of the indicated residues, are encompassed by the invention, with or without any additional modifications as described below. It will be understood that positions of amino acids, mutations or substitutions are numbered with reference to the amino acid sequence of DSG from Synechococcus elongatus (Anacystis nidulans, PCC 6301; SEQ ID NO:1) and that actual corresponding positions in DSG enzymes of other organisms may differ. Thus whenever reference is made to position X, it should be read as “a position corresponding to the amino acid at position X in SEQ ID No. 1”

TABLE 2

| Residue | Amino | Exemplary amino acid substitutions that |
|---|---|---|
| position # | acid present | increase or contribute to an increase in |
| in wild | in wild | desaturase activity when present at this |
| type DSG | type DSG | position |
| 8 | K | R |
| 13 | W | R |
| 16 | A | P |
| 30 | F | L |
| 32 | P | T |
| 69 | E | G, R, S, K, A, Y, T, L, N |
| 88 | H | Q |
| 96 | L | I |
| 104 | S | P |
| 129 | I | T, P, F, A, S, H, C, Q, V |
| 131 | A | V, S |
| 132 | R | K |
| 198 | L | F |
| 213 | S | R, P, H, Y, T |
| 214 | G | R, S, P, L, T, E |
| 225 | L | I |
| 240 | Q | R, A, K |

Exemplary amino acid changes which result in increased desaturase activity are shown in Table 3, together with the percentage increase in desaturase activity caused by each.

TABLE 3

| | | | | |
|---|---|---|---|---|
| | | 16:0 | 16:0 | |
| | | conversion | conversion | Fraction of |
| | | of DES9* | of DES9 | control |
| Strain name | Amino acid changes | (%) | control (%) | conversion |

Strains with single mutation

| | | | | |
|---|---|---|---|---|
| DES9*E69G | E69G | 67.2 | 21.4 | 3.1 |
| DES9*E69R | E69R | 69.3 | 32.0 | 2.2 |
| DES9*E69S | E69S | 39.2 | 20.5 | 1.9 |
| DES9*E69K | E69K | 42.9 | 20.5 | 2.1 |
| DES9*E69A | E69A | 46.6 | 20.5 | 2.3 |
| DES9*E69Y | E69Y | 56.7 | 32.0 | 1.8 |
| DES9*L96L | L96I | 53.4 | 21.4 | 2.5 |
| DES9*I129T | I129T | 62.5 | 30.2 | 2.1 |
| DES9*I129P | I129P | 51.3 | 20.5 | 2.5 |
| DES9*I129F | I129F | 69.2 | 21.4 | 3.2 |
| DES9*I129A | I129A | 57.5 | 32.0 | 1.8 |
| DES9*I129S | I129S | 65.5 | 32.0 | 2.0 |
| DES9*I129H | I129H | 44.3 | 20.5 | 2.2 |
| DES9*A131V | A131V | 55.0 | 21.4 | 2.6 |
| DES9*S213R | S213R | 54.8 | 20.5 | 2.7 |
| DES9*S213P | S213P | 27.6 | 20.5 | 1.3 |
| DES9*S213H | S213H | 53.8 | 32.0 | 1.7 |
| DES9*G214R | G214R | 64.9 | 30.2 | 2.1 |
| DES9*Q240R | Q240R | 70.1 | 21.4 | 3.3 |
| DES9*Q240A | Q240A | 63.0 | 32.0 | 2.0 |

Strains with more than one mutation

| | | | | |
|---|---|---|---|---|
| DES9*1 | W13R E69K | 55.2 | 21.4 | 2.6 |
| DES9*2 | F30L Q240K | 64.5 | 21.4 | 3.0 |
| DES9*3 | P32T I129T Q240R | 66.5 | 21.4 | 3.1 |
| DES9*4 | E69G V191I | 62.8 | 21.4 | 2.9 |
| DES9*5 | E69K A274P | 65.9 | 21.4 | 3.1 |
| DES9*6 | G175D S213P G214R I283K | 59.0 | 21.4 | 2.8 |
| DES9*7 | A16P L198F | 50.2 | 21.4 | 2.3 |
| DES9*8 | S213P K280R | 53.5 | 21.4 | 2.5 |
| DES9*9 | K8R S104P | 60.4 | 30.2 | 2.0 |
| DES9*10 | R132K S213Y | 61.4 | 30.2 | 2.0 |
| DES9*11 | E69R I129C A131S K159E G214S | 71.9 | 26.3 | 2.7 |
| DES9*12 | E69A I129C A131V S213P G214P | 78.7 | 26.3 | 3.0 |
| DES9*13 | E69T S213T G214L Q240R | 76.1 | 26.3 | 2.9 |
| DES9*14 | E69L I129Q A131S S213T G214T | 67.3 | 26.3 | 2.6 |
| | Q240A | | | |
| DES9*15 | E69R I129A G214R | 77.3 | 26.3 | 2.9 |
| DES9*21 | E69K R132K G214E | 79.2 | 26.3 | 3.0 |
| DES9*22 | E69K I129V A131V | 80.9 | 26.3 | 3.1 |
| DES9*23 | R132K G214R Q240R | 82.0 | 26.3 | 3.1 |
| DES9*24 | K8R H88Q R132K Q240K | 82.3 | 26.3 | 3.1 |
| DES9*25 | E69G S213Y | 72.4 | 26.3 | 2.8 |
| DES9*26 | E69N I129T L225I Q240R | 82.0 | 26.3 | 3.1 |

In some aspects of the invention, the mutant enzyme includes at least one mutation at the following positions: E69, S123, G124, I129, A131, S213, G214, and Q240. Exemplary changes at these residues include E69 to R or K or G; 1129 to T; S123 to R; G124 to R; A131 to V; and Q240 to R or K.

Exemplary single mutations include: Q240R and E69R.

Exemplary combinations of mutations include: E69R/Q240R; K8R/S104P; R132K/S213Y; E69/I129A/G214R; E69A/I129C/A131V/S213P/G124P; R132K/G214R/Q240R; and K8R/H88Q/R132K/Q240K.

DSG encoding sequences particularly suitable as starting material for modifications as described herein to increase enzymatic activity are any of the following (which may have one or more substitutions as described in Tables 2 and 3):
1) a nucleotide sequence encoding a polypeptide with an amino acid sequence having at least 50% sequence identity to the amino acid sequence of DSG, such as the sequences referred to in the above Table 1 as CYAN 2, CYAN 3, CYAN 4, CYAN 5, CYAN 6, CYAN 7, CYAN 8, CYAN 9, CYAN 10, CYAN 11, CYAN 12, CYAN 13, CYAN 14, CYAN 15, CYAN 16, CYAN 17, CYAN 18, CYAN 19, CYAN 20, CYAN 21, CYAN 22, CYAN 23, CYAN 24, CYAN 25, CYAN 26, CYAN 27, CYAN 28, CYAN 29, CYAN 30, CYAN 31, CYAN 32, CYAN 33, CYAN 34, CYAN 35, CYAN 36, CYAN 37, CYAN 38, CYAN 39, CYAN 40, CYAN 41, CYAN 42, CYAN 43, CYAN 44, CYAN 45, CYAN 46, CYAN 47, CYAN 48, CYAN 49, CYAN 50, CYAN 51, CYAN 52, CYAN 53, CYAN 54, CYAN 55, CYAN 56, CYAN 57, CYAN 58, CYAN 59, CYAN 60, CYAN 61, CYAN 62, CYAN 63, CYAN 64, CYAN 65, CYAN 66, CYAN 67, CYAN 68, CYAN 69, CYAN 70, CYAN 71, CYAN 72, CYAN 73, CYAN 74, CYAN 75, CYAN 76, CYAN 77, CYAN 78, CYAN 79, CYAN 80, CYAN 81, CYAN 82, CYAN 83, CYAN 84, CYAN 85, CYAN 86, CYAN 87, CYAN 88, CYAN 89, CYAN 90, CYAN 91, CYAN 92, CYAN 93, CYAN 94, CYAN 95, CYAN 96, CYAN 97, CYAN 98, CYAN 99, CYAN 100, CYAN 101, CYAN 102, CYAN 103, CYAN 104, CYAN 105, CYAN 106, CYAN 107, CYAN 108, CYAN 109, CYAN 110, CYAN 111, CYAN 112, CYAN 113, CYAN 114, CYAN 115, CYAN 116, CYAN 117, CYAN 118, CYAN 119, CYAN 120, CYAN 121, CYAN 122, CYAN 123, CYAN 124, CYAN 125, CYAN 126, CYAN 127, CYAN 128, CYAN 129, CYAN 130, CYAN 131, CYAN 132, CYAN 133, CYAN 134, CYAN 135, CYAN 136, CYAN 137, CYAN 138, CYAN 139, CYAN 140, CYAN 141, CYAN 142, CYAN 143, CYAN 144 or CYAN 146;
2) a nucleotide sequence encoding a polypeptide having Q at a position corresponding to DSG position 240 such as the sequences referred to in the above Table 1 as CYAN 2, CYAN 3, CYAN 4, CYAN 5, CYAN 6, CYAN 7, CYAN 8, CYAN 9, CYAN 10, CYAN 11, CYAN 12, CYAN 14, CYAN 16, CYAN 17, CYAN 18, CYAN 19, CYAN 20, CYAN 21, CYAN 22, CYAN 23, CYAN 24, CYAN 25, CYAN 27, CYAN 28, CYAN 29, CYAN 30, CYAN 31, CYAN 32, CYAN 33, CYAN 34, CYAN 35, CYAN 36, CYAN 37, CYAN 38, CYAN 39, CYAN 40, CYAN 41, CYAN 42, CYAN 43, CYAN 44, CYAN 45, CYAN 46, CYAN 47, CYAN 48, CYAN 49, CYAN 50, CYAN 51, CYAN 52, CYAN 53, CYAN 54, CYAN 55, CYAN 56, CYAN 57, CYAN 58, CYAN 59, CYAN 60, CYAN 61, CYAN 62, CYAN 63, CYAN 64, CYAN 65, CYAN 66, CYAN 67, CYAN 68, CYAN 70, CYAN 71, CYAN 72, CYAN 73, CYAN 74, CYAN 75, CYAN 76, CYAN 77, CYAN 78, CYAN 79, CYAN 81, CYAN 82, CYAN 83, CYAN 84, CYAN 85, CYAN 86, CYAN 87, CYAN 88, CYAN 89, CYAN 90, CYAN 91, CYAN 92, CYAN 93, CYAN 94, CYAN 95, CYAN 96, CYAN 97, CYAN 98, CYAN 99, CYAN 105, CYAN 113 or CYAN 176;
3) a nucleotide sequence encoding a polypeptide having E at a position corresponding to DSG position 69 such as the sequences referred to in the above Table 1 as CYAN 3, CYAN 17, CYAN 33, CYAN 45, CYAN 49, CYAN 50, CYAN 51, CYAN 52, CYAN 53, CYAN 54, CYAN 55, CYAN 58, CYAN 59, CYAN 66, CYAN 68, CYAN 70, CYAN 78, CYAN 96, CYAN 116, CYAN 124, CYAN 125, CYAN 129, CYAN 142, CYAN 143, CYAN 146, CYAN 179, CYAN 180 or CYAN 181;
4) a nucleotide sequence encoding a polypeptide having I at a position corresponding to DSG position 129 such as the sequences referred to in the above Table 1 as CYAN 3, CYAN 17, CYAN 20, CYAN 24, CYAN 25, CYAN 30, CYAN 32, CYAN 34, CYAN 36, CYAN 37, CYAN 38, CYAN 39, CYAN 40, CYAN 41, CYAN 42, CYAN 44, CYAN 46, CYAN 47, CYAN 48, CYAN 54, CYAN 55, CYAN 58, CYAN 61, CYAN 71, CYAN 72, CYAN 73, CYAN 76, CYAN 77, CYAN 83, CYAN 86, CYAN 87, CYAN 88, CYAN 89, CYAN 90, CYAN 91, CYAN 92, CYAN 93, CYAN 98, CYAN 100, CYAN 109, CYAN 110, CYAN 114, CYAN 115, CYAN 116, CYAN 117, CYAN 119, CYAN 120, CYAN 121, CYAN 122, CYAN 123, CYAN 124, CYAN 125, CYAN 126, CYAN 127, CYAN 128, CYAN 129, CYAN 130, CYAN 133, CYAN 134, CYAN 135, CYAN 138, CYAN 139, CYAN 140, CYAN 141, CYAN 142, CYAN 143, CYAN 144 or CYAN 146;
5) a nucleotide sequence encoding a polypeptide having A at a position corresponding to DSG position 131 such as the sequences referred to in the above Table 1 as CYAN 2, CYAN 3, CYAN 4, CYAN 5, CYAN 6, CYAN 7, CYAN 9, CYAN 11, CYAN 12, CYAN 13, CYAN 15, CYAN 16, CYAN 20, CYAN 22, CYAN 24, CYAN 25, CYAN 26, CYAN 30, CYAN 31, CYAN 32, CYAN 34, CYAN 35, CYAN 36, CYAN 37, CYAN 38, CYAN 39, CYAN 40, CYAN 41, CYAN 42, CYAN 44, CYAN 45, CYAN 46, CYAN 47, CYAN 48, CYAN 49, CYAN 50, CYAN 51, CYAN 52, CYAN 53, CYAN 54, CYAN 55, CYAN 56, CYAN 57, CYAN 58, CYAN 59, CYAN 60, CYAN 61, CYAN 62, CYAN 63, CYAN 65, CYAN 66, CYAN 67, CYAN 69, CYAN 71, CYAN 72, CYAN 73, CYAN 74, CYAN 76, CYAN 77, CYAN 80, CYAN 81, CYAN 82, CYAN 83, CYAN 85, CYAN 86, CYAN 87, CYAN 88, CYAN 89, CYAN 90, CYAN 91, CYAN 92, CYAN 93, CYAN 94, CYAN 95, CYAN 96, CYAN 98, CYAN 99, CYAN 100, CYAN 101, CYAN 102, CYAN 103, CYAN 104, CYAN 106, CYAN 107, CYAN 108, CYAN 109, CYAN 110, CYAN 111, CYAN 112, CYAN 114, CYAN 115, CYAN 116, CYAN 117, CYAN 118, CYAN 119, CYAN 120, CYAN 121, CYAN 122, CYAN 123, CYAN 124, CYAN 125, CYAN 126, CYAN 127, CYAN 128, CYAN 129, CYAN 130, CYAN 131, CYAN 132, CYAN 133, CYAN 134, CYAN 135, CYAN 136, CYAN 138, CYAN 139, CYAN 140, CYAN 141, CYAN 142, CYAN 143, CYAN 144, CYAN 146, CYAN 156 or CYAN 197;
6) a nucleotide sequence encoding a polypeptide having S at a position corresponding to DSG position 213 such as the sequences referred to in the above Table 1 as CYAN 2, CYAN 3, CYAN 7, CYAN 10, CYAN 11, CYAN 14, CYAN 15, CYAN 16, CYAN 17, CYAN 18, CYAN 19, CYAN 20, CYAN 22, CYAN 23, CYAN 24, CYAN 25, CYAN 26, CYAN 30, CYAN 31, CYAN 32, CYAN 33, CYAN 34, CYAN 35, CYAN 36, CYAN 37, CYAN 38, CYAN 39, CYAN 40, CYAN 41, CYAN 42, CYAN 43, CYAN 44, CYAN 45, CYAN 46, CYAN 47, CYAN 48, CYAN 49, CYAN 50, CYAN 51, CYAN 52, CYAN 53, CYAN 54, CYAN 55, CYAN 56, CYAN 57, CYAN 58, CYAN 59, CYAN 60, CYAN 61, CYAN 62, CYAN 63, CYAN 65, CYAN 66, CYAN 67, CYAN 68, CYAN 69, CYAN 71, CYAN 72, CYAN 73, CYAN 74, CYAN 75, CYAN 76, CYAN 77, CYAN 78, CYAN 79, CYAN 83, CYAN 84, CYAN 86, CYAN 87, CYAN 88, CYAN 89, CYAN 90, CYAN 91, CYAN 92, CYAN 93, CYAN 95, CYAN 96, CYAN 97, CYAN 98, CYAN 99, CYAN 100, CYAN 101, CYAN 102, CYAN 103, CYAN 104, CYAN 105, CYAN 106, CYAN 107, CYAN 108, CYAN 109, CYAN 110, CYAN 111, CYAN 112, CYAN 113, CYAN 114, CYAN 115, CYAN 118, CYAN 120, CYAN 121, CYAN 122, CYAN 126, CYAN 127, CYAN 128, CYAN 130, CYAN 131, CYAN 132, CYAN 133, CYAN 134, CYAN 135, CYAN 136, CYAN 139, CYAN 140, CYAN 142, CYAN 144, CYAN 146, CYAN 147, CYAN 148, CYAN 151, CYAN 153, CYAN 157, CYAN 158, CYAN 163, CYAN 165, CYAN 169, CYAN 170, CYAN 172, CYAN 177, CYAN 179, CYAN 180, CYAN 181, CYAN 182, CYAN 188, CYAN 190 or CYAN 192;
7) a nucleotide sequence encoding a polypeptide having G at a position corresponding to DSG position 214 such as the sequences referred to in the above Table 1 as CYAN 2, CYAN 4, CYAN 10, CYAN 11, CYAN 12, CYAN 14, CYAN 15, CYAN 16, CYAN 18, CYAN 19, CYAN 21, CYAN 22, CYAN 23, CYAN 26, CYAN 27, CYAN 28, CYAN 29, CYAN 35, CYAN 57, CYAN 60, CYAN 62, CYAN 63, CYAN 65, CYAN 66, CYAN 74, CYAN 79, CYAN 83, CYAN 97, CYAN 99, CYAN 100, CYAN 101, CYAN 102, CYAN 103, CYAN 104, CYAN 106, CYAN 107, CYAN 108, CYAN 111, CYAN 112, CYAN 115, CYAN 118, CYAN 121, CYAN 126, CYAN 127, CYAN 128, CYAN 130, CYAN 131, CYAN 132, CYAN 133, CYAN 134, CYAN 135, CYAN 136, CYAN 139, CYAN 140 or CYAN 144;
8) a nucleotide sequence encoding a polypeptide having E at a position corresponding to DSG position 69 and Q at a position corresponding to DSG position 240 such as the sequences referred to in the above Table 1 as CYAN 3, CYAN 17, CYAN 33, CYAN 45, CYAN 49, CYAN 50, CYAN 51, CYAN 52, CYAN 53, CYAN 54, CYAN 55, CYAN 58, CYAN 59, CYAN 66, CYAN 68, CYAN 70, CYAN 78 or CYAN 96;
9) a nucleotide sequence encoding a polypeptide having I at a position corresponding to DSG position 129 and Q at a position corresponding to DSG position 240 such as the sequences referred to in the above Table 1 as CYAN 3, CYAN 17, CYAN 20, CYAN 24, CYAN 25, CYAN 30, CYAN 32, CYAN 34, CYAN 36, CYAN 37, CYAN 38, CYAN 39, CYAN 40, CYAN 41, CYAN 42, CYAN 44, CYAN 46, CYAN 47, CYAN 48, CYAN 54, CYAN 55, CYAN 58, CYAN 61, CYAN 71, CYAN 72, CYAN 73, CYAN 76, CYAN 77, CYAN 83, CYAN 86, CYAN 87, CYAN 88, CYAN 89, CYAN 90, CYAN 91, CYAN 92, CYAN 93 or CYAN 98;
10) a nucleotide sequence encoding a polypeptide having A at a position corresponding to DSG position 131 and Q at a position corresponding to DSG position 240 such as the sequences referred to in the above Table 1 as CYAN 2, CYAN 3, CYAN 4, CYAN 5, CYAN 6, CYAN 7, CYAN 9, CYAN 11, CYAN 12, CYAN 16, CYAN 20, CYAN 22, CYAN 24, CYAN 25, CYAN 30, CYAN 31, CYAN 32, CYAN 34, CYAN 35, CYAN 36, CYAN 37, CYAN 38, CYAN 39, CYAN 40, CYAN 41, CYAN 42, CYAN 44, CYAN 45, CYAN 46, CYAN 47, CYAN 48, CYAN 49, CYAN 50, CYAN 51, CYAN 52, CYAN 53, CYAN 54, CYAN 55, CYAN 56, CYAN 57, CYAN 58, CYAN 59, CYAN 60, CYAN 61, CYAN 62, CYAN 63, CYAN 65, CYAN 66, CYAN 67, CYAN 71, CYAN 72, CYAN 73, CYAN 74, CYAN 76, CYAN 77, CYAN 81, CYAN 82, CYAN 83, CYAN 85, CYAN 86, CYAN 87, CYAN 88, CYAN 89, CYAN 90, CYAN 91, CYAN 92, CYAN 93, CYAN 94, CYAN 95, CYAN 96, CYAN 98 or CYAN 99;
11) a nucleotide sequence encoding a polypeptide having S at a position corresponding to DSG position 213 and Q at a position corresponding to DSG position 240 such as the sequences referred to in the above Table 1 as CYAN 2, CYAN 3, CYAN 7, CYAN 10, CYAN 11, CYAN 14, CYAN 16, CYAN 17, CYAN 18, CYAN 19, CYAN 20, CYAN 22, CYAN 23, CYAN 24, CYAN 25, CYAN 30, CYAN 31, CYAN 32, CYAN 33, CYAN 34, CYAN 35, CYAN 36, CYAN 37, CYAN 38, CYAN 39, CYAN 40, CYAN 41, CYAN 42, CYAN 43, CYAN 44, CYAN 45, CYAN 46, CYAN 47, CYAN 48, CYAN 49, CYAN 50, CYAN 51, CYAN 52, CYAN 53, CYAN 54, CYAN 55, CYAN 56, CYAN 57, CYAN 58, CYAN 59, CYAN 60, CYAN 61, CYAN 62, CYAN 63, CYAN 65, CYAN 66, CYAN 67, CYAN 68, CYAN 71, CYAN 72, CYAN 73, CYAN 74, CYAN 75, CYAN 76, CYAN 77, CYAN 78, CYAN 79, CYAN 83, CYAN 84, CYAN 86, CYAN 87, CYAN 88, CYAN 89, CYAN 90, CYAN 91, CYAN 92, CYAN 93, CYAN 95, CYAN 96, CYAN 97, CYAN 98, CYAN 99, CYAN 105 or CYAN 113;
12) a nucleotide sequence encoding a polypeptide having G at a position corresponding to DSG position 214 and Q at a position corresponding to DSG position 240 such as the sequences referred to in the above Table 1 as CYAN 2, CYAN 4, CYAN 10, CYAN 11, CYAN 12, CYAN 14, CYAN 16, CYAN 18, CYAN 19, CYAN 21, CYAN 22, CYAN 23, CYAN 27, CYAN 28, CYAN 29, CYAN 35, CYAN 57, CYAN 60, CYAN 62, CYAN 63, CYAN 65, CYAN 66, CYAN 74, CYAN 79, CYAN 83, CYAN 97 or CYAN 99;
13) a nucleotide sequence encoding a polypeptide having E at a position corresponding to DSG position 69, I at a position corresponding to DSG position 129 and Q at a position corresponding to DSG position 240 such as the sequences referred to in the above Table 1 as CYAN 3, CYAN 17, CYAN 54, CYAN 55 or CYAN 58;
14) a nucleotide sequence encoding a polypeptide having E at a position corresponding to DSG position 69, A at a position corresponding to DSG position 131 and Q at a position corresponding to DSG position 240 such as the sequences referred to in the above Table as CYAN 3, CYAN 45, CYAN 49, CYAN 50, CYAN 51, CYAN 52, CYAN 53, CYAN 54, CYAN 55, CYAN 58, CYAN 59, CYAN 66 or CYAN 96;
15) a nucleotide sequence encoding a polypeptide having E at a position corresponding to DSG position 69, S at a position corresponding to DSG position 213 and Q at a position corresponding to DSG position 240 such as the sequences referred to in the above Table as CYAN 3, CYAN 17, CYAN 33, CYAN 45, CYAN 49, CYAN 50, CYAN 51, CYAN 52, CYAN 53, CYAN 54, CYAN 55, CYAN 58, CYAN 59, CYAN 66, CYAN 68, CYAN 78 or CYAN 96;
16) a nucleotide sequence encoding a polypeptide having E at a position corresponding to DSG position 69, G at a position corresponding to DSG position 214 and Q at a position corresponding to DSG position 240 such as the sequences referred to in the above Table 1 as CYAN 66;
17) a nucleotide sequence encoding a polypeptide having I at a position corresponding to DSG position 129, A at a position corresponding to DSG position 131 and Q at a position corresponding to DSG position 240 such as the sequences referred to in the above Table 1 as CYAN 3, CYAN 20, CYAN 24, CYAN 25, CYAN 30, CYAN 32, CYAN 34, CYAN 36, CYAN 37, CYAN 38, CYAN 39, CYAN 40, CYAN 41, CYAN 42, CYAN 44, CYAN 46, CYAN 47, CYAN 48, CYAN 54, CYAN 55, CYAN 58, CYAN 61, CYAN 71, CYAN 72, CYAN 73, CYAN 76, CYAN 77, CYAN 83, CYAN 86, CYAN 87, CYAN 88, CYAN 89, CYAN 90, CYAN 91, CYAN 92, CYAN 93 or CYAN 98;
18) a nucleotide sequence encoding a polypeptide having I at a position corresponding to DSG position 129, S at a position corresponding to DSG position 213 and Q at a position corresponding to DSG position 240 such as the sequences referred to in the above Table 1 as AS CYAN 3, CYAN 17, CYAN 20, CYAN 24, CYAN 25, CYAN 30, CYAN 32, CYAN 34, CYAN 36, CYAN 37, CYAN 38, CYAN 39, CYAN 40, CYAN 41, CYAN 42, CYAN 44, CYAN 46, CYAN 47, CYAN 48, CYAN 54, CYAN 55, CYAN 58, CYAN 61, CYAN 71, CYAN 72, CYAN 73, CYAN 76, CYAN 77, CYAN 83, CYAN 86, CYAN 87, CYAN 88, CYAN 89, CYAN 90, CYAN 91, CYAN 92, CYAN 93 or CYAN 98;
19) a nucleotide sequence encoding a polypeptide having I at a position corresponding to DSG position 129, G at a position corresponding to DSG position 214 and Q at a position corresponding to DSG position 240 such as the sequences referred to in the above Table 1 as CYAN 83;
20) a nucleotide sequence encoding a polypeptide having A at a position corresponding to DSG position 131,S at a position corresponding to DSG position 213 and Q at a position corresponding to DSG position 240 such as the sequences referred to in the above Table 1 as CYAN 2, CYAN 3, CYAN 7, CYAN 11, CYAN 16, CYAN 20, CYAN 22, CYAN 24, CYAN 25, CYAN 30, CYAN 31, CYAN 32, CYAN 34, CYAN 35, CYAN 36, CYAN 37, CYAN 38, CYAN 39, CYAN 40, CYAN 41, CYAN 42, CYAN 44, CYAN 45, CYAN 46, CYAN 47, CYAN 48, CYAN 49, CYAN 50, CYAN 51, CYAN 52, CYAN 53, CYAN 54, CYAN 55, CYAN 56, CYAN 57, CYAN 58, CYAN 59, CYAN 60, CYAN 61, CYAN 62, CYAN 63, CYAN 65, CYAN 66, CYAN 67, CYAN 71, CYAN 72, CYAN 73, CYAN 74, CYAN 76, CYAN 77, CYAN 83, CYAN 86, CYAN 87, CYAN 88, CYAN 89, CYAN 90, CYAN 91, CYAN 92, CYAN 93, CYAN 95, CYAN 96, CYAN 98 or CYAN 99;
21) a nucleotide sequence encoding a polypeptide having A at a position corresponding to DSG position 131, G at a position corresponding to DSG position 214 and Q at a position corresponding to DSG position 240 such as the sequences referred to in the above Table 1 as CYAN 2, CYAN 4, CYAN 11, CYAN 12, CYAN 16, CYAN 22, CYAN 35, CYAN 57, CYAN 60, CYAN 62, CYAN 63, CYAN 65, CYAN 66, CYAN 74, CYAN 83 or CYAN 99;
22) a nucleotide sequence encoding a polypeptide having S at a position corresponding to DSG position 213, G at a position corresponding to DSG position 214 and Q at a position corresponding to DSG position 240 such as the sequences referred to in the above Table 1 as CYAN 2, CYAN 10, CYAN 11, CYAN 14, CYAN 16, CYAN 18, CYAN 19, CYAN 22, CYAN 23, CYAN 35, CYAN 57, CYAN 60, CYAN 62, CYAN 63, CYAN 65, CYAN 66, CYAN 74, CYAN 79, CYAN 83, CYAN 97 or CYAN 99;
23) a nucleotide sequence encoding a polypeptide having E at a position corresponding to DSG position 69, I at a position corresponding to DSG position 129, A at a position corresponding to DSG position 131 and Q at a position corresponding to DSG position 240 such as the sequences referred to in the above Table 1 as CYAN 3, CYAN 54, CYAN 55 or CYAN 58;
24) a nucleotide sequence encoding a polypeptide having E at a position corresponding to DSG position 69, I at a position corresponding to DSG position 129, S at a position corresponding to DSG position 213 and Q at a position corresponding to DSG position 240 such as the sequences referred to in the above Table 1 as CYAN 3, CYAN 17, CYAN 54, CYAN 55 or CYAN 58;
25) a nucleotide sequence encoding a polypeptide having E at a position corresponding to DSG position 69, I at a position corresponding to DSG position 129, G at a position corresponding to DSG position 214 and Q at a position corresponding to DSG position 240 such as the sequence referred to in the above Table 1 as CYAN 1;
26) a nucleotide sequence encoding a polypeptide having E at a position corresponding to DSG position 69, A at a position corresponding to DSG position 131, S at a position corresponding to DSG position 213 and Q at a position corresponding to DSG position 240 such as the sequences referred to in the above Table 1 as CYAN 66;
27) a nucleotide sequence encoding a polypeptide having E at a position corresponding to DSG position 69, A at a position corresponding to DSG position 131, G at a position corresponding to DSG position 214 and Q at a position corresponding to DSG position 240 such as the sequences referred to in the above Table 1 CYAN 66;
28) a nucleotide sequence encoding a polypeptide having E at a position corresponding to DSG position 69, S at a position corresponding to DSG position 213, G at a position corresponding to DSG position 214 and Q at a position corresponding to DSG position 240 such as the sequences referred to in the above Table 1 as CYAN 66;
29) a nucleotide sequence encoding a polypeptide having I at a position corresponding to DSG position 129, A at a position corresponding to DSG position 131, S at a position corresponding to DSG position 213 and Q at a position corresponding to DSG position 240 such as the sequences referred to in the above Table 1 as CYAN 83;
30) a nucleotide sequence encoding a polypeptide having I at a position corresponding to DSG position 129, A at a position corresponding to DSG position 131, G at a position corresponding to DSG position 214 and Q at a position corresponding to DSG position 240 such as the sequences referred to in the above Table 1 as CYAN 83;
31) a nucleotide sequence encoding a polypeptide having I at a position corresponding to DSG position 129, S at a position corresponding to DSG position 213, G at a position corresponding to DSG position 214 and Q at a position corresponding to DSG position 240 such as the sequences referred to in the above Table 1 as CYAN 83;
32) a nucleotide sequence encoding a polypeptide having A at a position corresponding to DSG position 131, S at a position corresponding to DSG position 213, G at a position corresponding to DSG position 214 and Q at a position corresponding to DSG position 240 such as the sequences referred to in the above Table 1 as CYAN 2, CYAN 11, CYAN 16, CYAN 22, CYAN 35, CYAN 57, CYAN 60, CYAN 62, CYAN 63, CYAN 65, CYAN 66, CYAN 74, CYAN 83 or CYAN 99;
33) a nucleotide sequence encoding a polypeptide having E at a position corresponding to DSG position 69, I at a position corresponding to DSG position 129, A at a position corresponding to DSG position 131, S at a position corresponding to DSG position 214 and Q at a position corresponding to DSG position 240 such as the sequences referred to in the above Table 1 as CYAN 3, CYAN 17, CYAN 54, CYAN 55, CYAN 58; or
34) a nucleotide sequence encoding a polypeptide having E at a position corresponding to DSG position 69, A at a position corresponding to DSG position 131, S at a position corresponding to DSG position 213, G at a position corresponding to DSG position 214 and Q at a position corresponding to DSG position 240 such as the sequences referred to in the above Table 1 as CYAN 66.
   The current invention also provides novel DSG proteins:
1) The natural variation of amino acids at a position corresponding to E69 of DSG does not include a G, A, Y or L at that position and accordingly novel DSG-like proteins are provided which have a E69G, E69A, E69Y or E69L amino acid substitution.
2) The natural variation of amino acids at a position corresponding to 1129 of DSG does not include a F at that position and accordingly novel DSG-like proteins are provided which have a 1129F amino acid substitution.
3) The natural variation of amino acids at a position corresponding to 5213 of DSG does not include a H or Y at that position and accordingly novel DSG-like proteins are provided which have a S213H or S213Y amino acid substitution.
4) The natural variation of amino acids at a position corresponding to G214 of DSG does not include a L at that position and accordingly novel DSG-like proteins are provided which have a E69G, E69A, E69Y or E69L amino acid substitution.
5) The natural variation of amino acids at a position corresponding to Q240 of DSG does not include a R, A or K at that position and accordingly novel DSG-like proteins are provided which have a Q240R, Q240A or Q240K amino acid substitution.

Also provided are natural, unprocessed oils, particularly non-dehydrogenated oils, especially oil obtained from oilseed rape plants, such as Brassica napus or Brassica juncea, particularly canola-quality oil, which contain less than 3% saturated fatty acids.

The invention further provides a method for producing food, feed, or an industrial product comprising obtaining a plant or a part thereof, as herein described, including plants comprising a foreign recombinant gene encoding a cyanobacterial derived DSG like or DES9 like mutant and preparing the food, feed or industrial product from the plant or part thereof. The food or fees may be oil, meal, grain, starch, flour or protein; or the industrial product may be biofuel, fiber, industrial chemicals, a pharmaceutical or a nutraceutical.

In addition, the DSG mutants of the invention may be further modified, for example, by the addition (or optimization, enhancement, etc.) of sequences which target or direct the DSG to a particular location or locations within the host cell in which they are expressed. Exemplary targeting modifications include but are not limited to the incorporation of one or more signal peptides (signal sequences, leader sequences, leader peptides, etc.) at the N-terminus of a DSG. The signal sequences may be from heterologous “Type I” proteins. Generally, a signal peptide is a short (e.g. about 5-30 amino acids) peptide present at the N-terminus of the majority of newly synthesized proteins that are destined towards a secretory pathway. These sequences direct a protein, for example, to reside inside certain organelles (the endoplasmic reticulum, golgi or endosomes), to be secreted from the cell, or to be inserted into one or more cellular membranes. The signal peptide may be cleaved from the protein after it reaches its destination. However, in some instances (e.g. for “Type II” proteins), the “targeting sequence” is a heterologous first transmembrane domain, which biochemically resembles a signal sequence but is not cleaved from the protein. These modifications may be added to a polypeptide sequence of interest, or may replace sequences of interest in the polypeptide (e.g. may replace “native” sequences”) to provide enhanced performance compared to the native sequence.

Thus, it may be desirable for the mutant prokaryotic DSG of the invention to be located in the membrane of a eukaryotic host cell, or in a particular subcellular organelle of a eukaryotic host cell. In one aspect, when the host cell is a cell of an oil seed plant, it may be desirable for the DSG to be present and active in the endoplasmic reticulum (ER) of the host cell.

Other sequences may also be appended to the initial, prototype DSG sequence that is modified. For example, various “retention” sequences are known in the art and may be added to facilitate or enable retention of the protein at a location of interest. Exemplary sequences include but are not limited to: so-called “classical” amino terminal sequences KDEL (SEQ ID NO: 6) and HDEL (SEQ ID NO: 7), and variants thereof, and -GKSKIN (SEQ ID NO: 5), which function in ER retention; cytoplasmic retention sequences; membrane retention sequences; cell surface retention sequences (e.g. clusters of 6-7 basic amino acids); etc.

Those of skill in the art will recognize that various other modified forms (variants or derivatives) of the amino acid sequences disclosed herein may be made, and the invention encompasses all such variants/derivatives, as long as the resulting molecule retains a desired level of desaturase activity as described herein. For example, the recombinant DSGs may also contain, in addition to those disclosed herein, other suitable mutations or alterations such as various additional amino acid substitutions, which may be conservative or non-conservative amino acid substitutions, and/or additions to or deletions from the sequence, may be included in and tolerated by the recombinant enzymes, while still allowing the further mutated recombinants to retain a desired or useful level of desaturase activity. Generally, such further mutated enzymes will retain at least about 50, 55, 60, 65, 70, 75, 80, 85, 90, 95 or 100% (or more) of the desaturase activity displayed by the recombinant enzyme from which they are derived (i.e. the recombinant that serves as the basis or prototype for further mutation). In some cases, the desaturase activity of the further mutated DSG may be greater than that of the recombinant enzyme from which it is derived. Exemplary additional mutations include but are not limited to: changes which introduce or eliminate sequences susceptible to proteolysis; various “tagging” sequences which may be used to identify and/or to isolate the recombinants, e.g. His tags, HA tag etc changes which increase or decrease solubility, e.g. water solubility, lipid and/or membrane solubility, etc.; incorporation of one or more so-called “non-natural” amino acids; etc. All such possible variants of derivatives of the DSG recombinants disclosed herein are encompassed by the present invention.

The enzymes may be pre- or post-translationally modified, either non-enzymatically or enzymatically by enzymes in the cell, and this may occur naturally within the cell or may be introduced intentionally after translation of the protein. Exemplary post-translational modifications include but are not limited to: attachment of various biochemical functional groups such as acetate, phosphate, various lipids and carbohydrates, etc.; changes to the chemical nature of one or more amino acids (e.g. citrullination); structural changes (e.g. formation of disulfide bridges); non-enzymatic deamidation of susceptible Asn and/or Gln residues; amidation; removal of leader sequences; and the like. Post translational modifications (PTMs) involving addition of smaller chemical groups include but are not limited to: acylation, e.g. O-acylation (esters); N-acylation (amides); S-acylation (thioesters); acetylation, the addition of an acetyl group, either at the N-terminus or at lysine residues; alkylation, the addition of an alkyl group, e.g. methyl, ethyl; methylation, the addition of a methyl group, usually at lysine or arginine residues; amide bond formation; amidation at the C-terminus; amino acid addition (e.g. arginylation, polyglutamylation, polyglycylation, etc.); butyrylation; gamma-carboxylation; glycosylation, the addition of a glycosyl group to e.g. arginine, asparagine, cysteine, hydroxylysine, serine, threonine, tyrosine, or tryptophan; malonylation; hydroxylation; oxidation; phosphate ester (O-linked) or phosphoramidate (N-linked) formation phosphorylation, the addition of a phosphate group, usually to serine, threonine, and/or tyrosine (O-linked), or histidine (N-linked); adenylylation; propionylation; pyroglutamate formation; S-glutathionylation; S-nitrosylation; succinylation addition of a succinyl group to lysine; sulfation, the addition of a sulfate group to a tyrosine; glycation, the addition of a sugar molecule to a protein without the controlling action of an enzyme; biotinylation, acylation of conserved lysine residues with a biotin appendage; pegylation, etc.

The enzymes may also include various labels which are known in the art, for example: radioactive isotopes may be incorporated; biotin may be added; the mutants may be conjugated to enzymes such as horseradish peroxidase (HRP), alkaline phosphatase (AP), glucose oxidase and β-galactosidase; various fluorescent, chemiluminescent or phosphorescent labels may be attached (e.g. organic dyes such as flurorisothiocyanate (FITC), tetramethylrho-damine isothiocyanate (TRITC) and various rhodamine dyes, DyLight Fluors for labeling amine or sulfhydryl groups, etc.); biological fluorophore may be used (e.g. Green fluorescent protein (GFP), R-Phycoerythrin; nanoscale-sized (2-50 nm) semiconductors known as “Quantum dots”; various Expressed Sequence Tags (ESTs); etc.

Fragments of the recombinant protein sequences described herein are also encompassed. Such fragments generally retain at least at least about 50, 55, 60, 65, 70, 75, 80, 85, 90, 95 or 100% (or more) of the desaturase activity displayed by the recombinant enzyme from which they are derived. Fragments may be generated by cleavage of amino acid residues from either or both of the amino- and carboxy termini after translation. Alternatively, “fragments” may be generated by deleting nucleotides that correspond to the amino acid sequence(s) which are to be eliminated so that the protein is translated as a “fragment”. “Fragments” may also refer to polypeptides with internal deletions.

In general, the amino acid sequences of such variants are at least about 50, 55, 60, 65, 70, 75, 80, 85, 90, 95, 96, 97, 98, or 99% identical to the recombinant sequence from which it is derived, or, for fragments, to a contiguous portion of a sequence of the same. In addition a “further modified” enzyme will generally retain at least about 50, 55, 60, 65, 70, 75, 80, 85, 90, 95 or 100% (or more) of the desaturase activity displayed by the recombinant enzyme from which it is derived. However, in some cases the modified forms of the mutants need not possess any particular level of desaturase activity since the modification (e.g. labeling or tagging) may be done for purposes other than to obtain an optimally active enzyme, e.g. to purify and sequence an enzyme, to locate an enzyme within a cell; etc.

The recombinant enzymes of the invention display increased or enhanced activity, compared to the wild type enzyme from which they are derived. By “enhanced” or “increased” activity, we mean that the activity of the enzyme with respect to catalyzing a chemical reaction (such as conversion of substrate such as a saturated fatty acid to product such as an unsaturated fatty acid) increases at least about 10, 15, 20, 25, 30, 35, 40, 45, 50, 55, 60, 65, 70, 75, 80, 85, 90, 95 or even 100%, compared to a suitable control, e.g. a wildtype enzyme measured under substantially the same reaction conditions. Alternatively, the increase may be expressed as a fold increase, e.g. the activity may be increased 2, 3, 4, 5, 6, 7, 8, 9 or 10-fold or more (e.g. 20, 25, 30, 35, 40, 45, 50, 55, 60, 65, 70, 75, 80, 85, 90, 95 or even 100 fold), compared to the activity of the native, unmodified enzyme. Generally, (e.g. see Table 1), the fold increase is at least about 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In the present invention, this “increase” may be due to any of several factors, e.g. to increased affinity for the substrate, decreased affinity for the product, increased turnover number, increased stability of the enzyme, etc.

### Nucleic Acids and Vectors

The present invention further provides isolated nucleic acid molecules and their complements that contain genetic sequences (genes) which encode an amino acid sequence with at least about 65, 70, 75, 80, 85, 90, 95, 96, 97, 98 or 99% sequence identity, with the protein/polypeptide sequences disclosed herein. Exemplary encoding nucleotide sequences are provided, but those of skill in the art will recognize that, due to the redundancy of the genetic code, other nucleotide sequences may also encode the same protein/polypeptide.

The gene sequences that encode the recombinant prokaryotic enzymes are destined to be expressed in transgenic eukaryotic host cells. Thus, the prokaryotic genes are operably linked to a promoter that is suitable for expression of the gene within a eukaryote. If the eukaryotic host is an animal or animal cell, suitable promoters include but are not limited to: bovine beta-lactoglobulin, chicken β-actin promoter, etc. if the eukaryotic host is a plant or plant cell, suitable promoters include but are not limited to: phaseolin, napin, 2S2 promoters the oilseed rape napin promoter (U.S. Pat. No. 5,608,152), the Vicia faba USP promoter (Baeumlein et al., MoI Gen Genet, 1991, 225 (3):459-67), the Arabidopsis oleosin promoter (WO 98/45461), the Phaseolus vulgaris phaseolin promoter (U.S. Pat. No. 5,504,200), the Brassica Bce4 promoter (WO 91/13980) or the legumine B4 promoter (LeB4; Baeumlein et al., 1992, Plant Journal, 2 (2):233-9), and promoters which bring about the seed-specific expression in monocotyledonous plants such as maize, barley, wheat, rye, rice and the like. Suitable noteworthy promoters are the barley Ipt2 or Ipt1 gene promoter (WO 95/15389 and WO 95/23230) or the promoters from the barley hordein gene, the rice glutelin gene, the rice oryzin gene, the rice prolamine gene, the wheat gliadine gene, the wheat glutelin gene, the maize zeine gene, the oat glutelin gene, the sorghum kasirin gene or the rye secalin gene, which are described in WO 99/16890. Also suitable promoters are those described in WO 2010/00708 or in WO 2010/057620 or WO2010/060609. Other useful promoters include the nopaline synthase, mannopine synthase, and octopine synthase promoters, which are carried on tumor-inducing plasmids of Agrobacterium tumefaciens; the cauliflower mosaic virus (CaMV) 19S and 35S promoters; the enhanced CaMV 35S promoter; the Figwort Mosaic Virus 35S15 promoter; the light-inducible promoter from the small subunit of ribulose-1,5-bisphosphate carboxylase (ssRUBISCO); the EIF-4A promoter from tobacco (Mandelet al. (1995) Plant Mol. Biol. 29:995-1004); corn sucrose synthetase; corn alcohol dehydrogenase I; corn light harvesting complex; corn heat shock protein; the chitinase promoter from Arabidopsis; the LTP (Lipid Transfer Protein) promoters; petunia 20 chalcone isomerase; bean glycine rich protein 1; potato patatin; the ubiquitin promoter; and the actin promoter. Useful promoters are preferably seed-selective, tissue selective, or inducible.

Other elements which enhance, control or optimize transcription and/or translation of the recombinant enzyme within the transgenic host include but are not limited to: various enhancer elements, e.g. various cis-acting elements within the regulatory regions of the DNA, trans-acting factors that include transcription factors, etc. One of more of these may also be included in the nucleic acid that contains the recombinant gene that is to be expressed in the host.

The nucleic acid molecules described herein may be modified, for example, by codon optimization to facilitate expression in heterologous cells. This type of modification changes or alters the nucleotide sequence that encodes a protein of interest to use, throughout the sequence, codons that are more-commonly used in the transgenic expression host cell. In addition, changes may be made to the nucleotide sequence that encodes the protein to adjust the relative concentration of A/T and G/C base pairs to ratios that are more similar to those of the expression host.

In addition, nucleotide sequences encoding the mutants of the invention may be further modified to encode other sequences such as those described above as being beneficial or desirable for inclusion in the modified proteins of the invention, e.g. sequences which target or direct the protein to a particular location or locations within the expression host cell, etc.

The invention also encompasses vectors that comprise the nucleic acid sequences described herein. “Vector” refers broadly to any plasmid or virus encoding an exogenous nucleic acid. (However, the term may also be construed to include non-plasmid and non-viral compounds which facilitate transfer of nucleic acid into virions or cells, such as, for example, polylysine compounds and the like.) The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art. Examples of viral vectors include, but are not limited to recombinant vaccinia, adeno-, retro-, adeno-associated, avian pox and other viral vectors. Examples of non-viral vectors include, but are not limited to, liposomes, polyamine derivatives of DNA, and the like.

### Transgenic Host Cells and Organisms, and Products Produced by the Same

The invention further encompasses transgenic host cells and transgenic host organisms. The transgenic hosts have been genetically engineered using molecular biology techniques to contain nucleic acid molecules which encode at least one of the recombinant proteins described herein. Within the transgenic host, the nucleic acid molecule is transcribed into mRNA which is then translated into a recombinant protein with the desired optimized activity. As noted above, the recombinant protein is derived from a heterologous protein that is not expressed in the transgenic host in nature. The transgenic hosts are generally eukaryotic and may include animal, plant and fungal hosts.

The invention provides methods of decreasing (reducing, lowering, etc.) the amounts (levels, concentrations, etc.) of saturated fatty acids in such transgenic hosts, and in products made by of from such transgenic hosts. The methods involve genetically engineering host cells to contain and express one or more heterologous recombinant desaturase enzymes as described herein. In some embodiments the levels of saturated fatty acids are reduced by a value that is in the range of at least about 25 to about 75%, and usually from at least about 35% to about 65%, or from at least about 45% to about 55%, compared to a suitable control cell, and the reduction may be at least about 50%. For example, the level of 16:0 saturated fats in a transgenic cell/organism/product of the invention will generally be less than about 7%, or less than about 4%, or even less than about 3% in seed. Those of skill in the art are familiar with the concept of suitable controls, which in this case would generally be a comparable cell (or product) that had not been genetically engineered to contain and express a mutant DSG as described herein. A comparable cell would generally be a cell of a similar type, e.g. a eukaryotic cell from the same genus and species (and/or from the same sub-species or strain, as appropriate), that is tested under the same or similar, or substantially the same or similar) conditions. The cell may be a “wild type” cell, or the cell may be a cell that has been cultured in vivo or in vitro. Further, to carry out a comparison, those of skill in the art will recognize that a sufficient number of data points must be obtained so that the results of the comparison are statistically significant. Methods of designing and carrying out such experiments, and analyzing the results are known.

### Animal Hosts

The invention encompasses transgenic animal cells or organisms that are genetically engineered to contain and express nucleic acids encoding at least one recombinant protein described herein.

Those of skill in the art are familiar with methods of genetically modifying animal cells and animals. For example: by injecting DNA into embryos then implanting the embryos in females; by DNA microinjection, e.g. by injection of a transgene of interest into the pronucleus of a reproductive cell (such as an egg), growth of the embryo in vitro until suitable to transfer into a suitable female animal; via retrovirus-mediated gene transfer to transfer genetic material into the host cell, resulting in a chimera (an organism comprising tissues or parts of diverse genetic constitution) and inbreeding the chimera until homozygous transgenic offspring are born; or by embryonic stem cell-mediated gene transfer, involving insertion of the gene of interest into totipotent stem cells, growth embryo, resulting in a chimeric animal

Animals and animal products that may be genetically modified to contain and express the desaturase enzymes of the invention include but are not limited to: turkey, chicken, fish, pig, cow, goat etc.

### Fungal Hosts

The invention also encompasses transgenic fungal cells that are genetically engineered to contain and express nucleic acids encoding at least one recombinant protein described herein.

### Plant Hosts

One aspect of the invention involves the generation of transgenic plants and/or transgenic plant cells which contain and express at least one nucleic acid as described herein. Methods of producing transgenic plants are well known to those of ordinary skill in the art. Transgenic plants can be produced by a variety of different transformation methods including, but not limited to, electroporation; microinjection; microprojectile bombardment, also known as particle acceleration or biolistic bombardment, e.g. using needle-like crystals (“whiskers”) of silicon carbide; viral-mediated transformation; Agrobacterium-, Rhizobium-, Mesorhizobium- and Sinorffizobium-mediated transformation. See, for example, U.S. Pat. Nos. 5,405,765; 5,472,869; 5,538,877; 5,538,880; 5,550,318; 5,641,664; 5,736,369; 5,736369; and US patent applications 2005/0289672 and 2005/0289667; each of which is expressly incorporated herein by reference in entirety.

Exemplary plants or plant cells that may be utilized in the practice of the invention include but are not limited to: oil seed plants, canola, safflower, camelina, soybean, corn, sunflower, peanut, sesame, cotton rice, wheat, Brassica oilseed plants including Brassica juncea, Brassica napus, Brassica carinata, Brassica nigra, Brassica rapa or Brassica campestris, Camelina spp., etc.

In some instances, the plants are “oil seed” plants. Generally, oil seed plants (which may be trees) are cultivated so that oil, especially edible oil, can be produced from the seeds, nuts, tubers, etc. of the plants. Exemplary oil seed plants include but are not limited to: coconut, corn, cotton, olive, palm, peanut (ground nut), various rapeseed plants including canola, safflower, sesame, flax, soybean, sunflower, and the like. Various plant species that produce nuts from which oils are extracted may also be employed, including those that produce hazelnuts (e.g. from the common hazel), almond, beech (e.g. which produce Fagus sylvatica nuts), cashew macadamia, mongongo (or manketti, seeds of the Schinziophyton rautanenii tree), pecan, pine, pistachio, walnut, etc. Various citrus plants and trees produce seeds which are used to prepare edible oils, e.g. lemon, orange oil, grapefruit, sea-buckthorn, etc. Various melons and gourds may be utilized, e.g. watermelon (e.g. Citrullus vulgaris), members of the Cucurbitaceae family including gourds, melons, pumpkins, and squashes; the bitter gourd (Momordica charantia), bottle gourd (e.g. Lagenaria siceraria), buffalo gourd (Cucurbita foetidissima), butternut squash (e.g. Cucurbita moschata), egusi (Cucumeropsis mannii naudin, pumpkin, etc. Other plants and/or trees that may be utilized include borage (e.g. Borago officinalis), blackcurrant, evening primrose (e.g. Oenothera biennis), açai (e.g. any of several species of the Açai palm (Euterpe), black seed (e.g. from Nigella sativa), blackcurrant (e.g. Ribes nigrum), flax (linseed, e.g. Linum usitatissimwn), carob, amaranth (e.g. from Amaranthus emeritus and Amaranthus hypochondriacus), apricot, apple, argan (e.g. from Argania spinosa), avocado, babassu r.g. Attalea speciosa), the seeds of Moringa oleifera, from which “ben” oil is extracted, species of genus Shorea, cape chestnut, the cacao plant, cocklebur (e.g. species of genus Xanthium), poppy, the Attalea cohune (cohune palm), coriander, date, Irvingia gabonensis, Camelina sativa, grape, hemp, Ceiba pentandra, Hibiscus cannabinus, Lallemantia iberica, Trichilia emetica, Sclerocarya birrea, meadowfoam, mustard, nutmeg (e.g. from cogeners of genus Myristica), okra (e.g. Abelmoschus esculentus), papaya, perilla, persimmon (e.g. Diospyros virginiana), Caryocar brasiliense, pili nut (e.g. Canarium ovatum), pomegranate (e.g. Punica granatum), prune quinoa, ramtil (e.g. several species of genus Guizotia abyssinica (Niger pea), rice, Prinsepia utilis, shea, Sacha inchi, sapote (e.g. Jessenia bataua), arugula (e.g. Eruca sativa), tea (Camellia), thistle (e.g. Silybum marianum), Cyperus esculentus, tobacco (e.g. Nicotiana tabacum and other Nicotiana species), tomato, and wheat, among others.

The plants of the invention thus include at least one recombinant desaturase as described herein, expressed in at least one location or tissue of the plant. As a result, at least one portion of the plant (i.e. at least one tissue or type of tissue, or at least one part of the plant) contains a lower amount or percentage of saturated fatty acids and/or a higher amount or percentage of unsaturated fatty acids than a native, control non-transgenic plants (non-transgenic plants meaning plants that have not been genetically modified as described herein; they may have been otherwise genetically modified). As used herein “plant” or “plant parts” or “plant tissue” includes any part of a plant, e.g. stems, leaves, roots, blossoms, seeds, fruit, nuts, berries, reproductive organs, embryonic tissue, individual cells, plants cells cultured in vitro, etc. Progeny of the transgenic plants of the invention are also encompassed.

In some aspects, the invention provides products produced by plants or from plants or parts of plants, for example, oils produced from the seeds or nuts of the transgenic plants. Exemplary oils of the invention include but are not limited to: Coconut oil, Corn oil, Cottonseed oil, Olive oil, Palm oil, Peanut oil (Ground nut oil), Rapeseed oil (including Canola oil) Safflower oil, Sesame oil, Soybean oil, and Sunflower oil. Various nut oils are also contemplated, including but not limited to: Almond oil, Beech nut oil, Cashew oil, Hazelnut oil, Macadamia oil, Mongongo nut oil (or manketti oil), Pecan oil, Pine nut oil, Pistachio oil, and Walnut oil. Various Cctrus oils are also contemplated, including but not limited to: Grapefruit seed oil, Lemon oil, Orange oil, and sea-buckthorn oil. Oils from melon and gourd seeds are also contemplated, including but not limited to: Cucurbitaceae oils from e.g. gourds, melons, pumpkins, and squashes such as Watermelon seed oil, Bitter gourd oil, Bottle gourd oil, Buffalo gourd oil, Butternut squash seed oil, Egusi seed oil, and Pumpkin seed oil, Various other plant-derived oils are also encompassed by the invention, including but not limited to: Açai oil, Arabidopsis oil, Black seed oil, Blackcurrant seed oil, Borage seed oil, Evening primrose oil, Flaxseed oil (linseed oil), Carob seed pods, Apricot oil, Apple seed oil, Argan oil, Avocado oil, Babassu oil, Ben oil, Borneo tallow nut oil, Cape chestnut oil, Carob pod oil (Algaroba oil), Cocoa butter, Cocklebur oil, Cohune oil, Coriander seed oil Date seed oil, Dika oil, False flax oil Grape seed oil, Hemp oil, Kapok seed oil, Kenaf seed oil, Lallemantia oil, Mafura oil, Manila oil, Meadowfoam seed oil, Mustard oil (pressed), Poppyseed oil, Nutmeg butter, Okra seed oil, Papaya seed oil, Perilla seed oil, Persimmon seed oil, Pequi oil, Pili nut oil, Pomegranate seed oil, Prune kernel oil, Quinoa oil, Ramtil oil, Rice bran oil Royle oil, Sacha inchi oil, Sapote oil, Seje oil, Shea butter, Taramira oil, Tea seed oil (Camellia oil), Thistle oil, Tigernut oil (or nut-sedge oil) Tobacco seed oil, Tomato seed oil, and Wheat germ oil, etc.

Also provided are natural, unprocessed oils, particularly non-hydrogenated oils, especially oil obtained from oilseed rape plants, such as Brassica napus or Brassica juncea, particularly canola-quality oil, which contain less than 3% saturated fatty acids.

The invention further provides a method for producing food, feed, or an industrial product comprising obtaining a plant or a part thereof, as herein described, including plants comprising a foreign recombinant gene encoding a cyanobacterial derived DSG like or DES9-like mutant and preparing the food, feed or industrial product from the plant or part thereof. The food or fees may be oil, meal, grain, starch, flour or protein; or the industrial product may be biofuel, fiber, industrial chemicals, a pharmaceutical or a nutraceutical.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Examples

### Example 1

Directed evolution of a cyanobacterial desaturase for increased activity in yeast, and application of the new enzymes to reduce saturated fatty acids in Arabidopsis seed.

### Summary

Plant oilseeds are a major source of nutritional oils. Their fatty acid composition, especially the proportion of saturated and unsaturated fatty acids, has important effects on human health. Because intake of saturated fats is correlated with the incidence of cardiovascular disease and diabetes, a goal of metabolic engineering is to develop oils low in saturated fatty acids. Palmitic acid (16:0) is the most abundant saturated fatty acid in the seeds of many oilseed crops. Fatty acid desaturases from prokaryotes are a largely untapped resource for reducing this 16:0 content in seed, in part because there are many obstacles to successfully expressing a prokaryotic gene in eukaryotes.

We have surmounted these obstacles by modification of DSG, a membrane-bound, fatty acid Δ9 desaturase cloned from Synechococcus elongatus (Anacystis nidulans, PCC 6301), for successful expression both in yeast and in Arabidopsis seed. Expression of DSG converts 16:0 to 16:1Δ9, and 18:0 to 18:1Δ9. Desaturase activity was initially low in both model systems, so we used a commercial service to optimize the open reading frame of the desaturase for eukaryotic expression, and we added a C-terminal endoplasmic reticulum retention signal at the same time: activity only modestly increased for these constructs, which we termed DES9. We therefore developed a directed evolution strategy based on the observation that DES9 expression only very poorly complements the unsaturated-fatty-acid auxotrophy of the yeast strain mutant in ole1, the only fatty acid desaturase of Saccharomyces cerevisiae. After mutagenesis, a number of DES9 variants (DES9*) were isolated and characterized, which were much more active in desaturating the yeast fatty acids than the parent enzyme.

We expressed selected DES9* in Arabidopsis and Camelina using a seed-specific promoter, and have identified plants with greatly reduced levels of saturated fatty acids. DES9* were much more effective in reducing the levels of 16:0 and 18:0 in Arabidopsis and Camelina seed than the original DES9 gene. In Arabidopsis, expression of some DES9* reduced the 16:0 content of the seed from the 8% found in wild-type seed to about 3%; the 18:0 was reduced from 2.5% to less than 1.5%. The products of desaturation were evident in these seed as well: 16:1 (about 0.5% in wild-type Arabidopsis) increased to as much as 10%, and 18:1 increased from 15% to 20% in some transgenic lines. In Camelina, expression of some DES9* reduced the 16:0 content of the seed from the 6.7% found in wild-type seed to about 0.6%, a 90% reduction. The effect on 18:0 in Camelina was also pronounced; the 2.4% level of 18:0 found in wild-type was reduced to one-sixth of that value, 0.4% for a DES9*transformed line. Similar to the Arabidopsis results, in Camelina 16:1 increased from 0.2% to 7.7% and 18:1 increased from 10% to 17%. These experiments demonstrate that directed evolution using a novel strategy based on yeast complementation can produce proteins that are much more active desaturases in oilseeds, with utility for modifying plant oils. Expression of these DES9* can reduce the level of saturated fatty acids in the oil, significantly improving its nutritional value and increase the level of unsaturated fatty acids in the oil.

### Introduction

### Palmitate Metabolism

Palmitate (16:0) is the most abundant saturated fatty acid in the seeds of canola (4% of total fatty acids), soybean (11%), and sunflower, and also in the model oilseed plant Arabidopsis, where 16:0, 18:0, and 20:0 occur in a ratio of 6:2:1. Both 16:0 and 18:0 fatty acids are synthesized as ACP thioesters in the Arabidopsis plastid. Whereas 18:0 is efficiently desaturated to 18:1 before export from the plastid and can undergo further desaturation in the endoplasmic reticulum (ER), saturated fatty acids exported from the chloroplast as CoA esters are largely incorporated into lipids without undergoing desaturation.

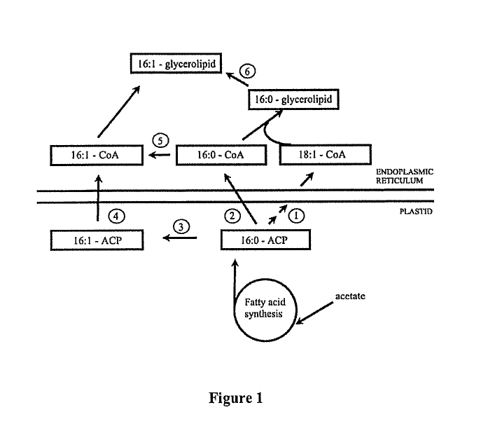

The current understanding of oilseed metabolism, e.g. the processes of synthesis, transport, and incorporation of 16:0 into triacylglycerols, where most of the oil in mature seeds occurs, is illustrated in FIG. 1. In plants fatty acids can be desaturated when bound to acyl carrier protein (ACP), to coenzyme A (CoA), or to a glycerol molecule as an acyl lipid. Plant enzymes that specifically desaturate ACP-bound fatty acids are found in plastids, where they introduce the first double bond into saturated fatty acids. In plants and cyanobacteria, many desaturation reactions use glycerolipid molecules as substrates, producing double bonds in fatty acids that are already incorporated into lipids. The final desaturase class, acyl-CoA desaturases, is found in animal and fungal cells, where they desaturate fatty acids bound to CoA. Plant desaturases may also act on acyl-CoA substrates.

An important consideration in efforts to reduce levels of 16:0 in plant tissues is that the fatty acid is a precursor to multiple pathways of plant metabolism, including being a significant component of sphingolipids, of waxes, and of cuticle structure. The fatty acid 16:0 is also required for regulatory palmitoylation of proteins. It is known that very low 16:0 levels are harmful to plant growth and reproduction.

Metabolic processes associated with oil production, including elongation and desaturation of fatty acids, TAG synthesis, and oil body formation are localized to the endoplasmic reticulum (ER). Because TAG in oil bodies is largely sequestered from the other metabolic activities, desaturation of 16:0 glycerolipids in the ER may limit disruption of the 16:0-CoA pool and thus minimize potential negative effects of lowered 16:0. In addition, heterologous expression using a robust seed-specific promoter should confine changes in fatty acid composition to the seed.

### Challenges to DSG Activity

In choosing a desaturase for these experiments, we considered which substrates are used by various desaturases. We chose to express the glycerolipid desaturase, DSG, from the cyanobacterium Synechococcus elongatus PCC 6301 (in the literature, also called Anacystis nidulans PCC 6301) in Arabidopsis seed. There are a number of potential obstacles to the use of DSG in oilseed modification, having to do with the nucleotides encoding the protein sequence, targeting of the protein within the cell, availability of the desaturase substrate in eukaryotes, and finally the source of electrons that the desaturase requires to introduce the double bond.

The genetic code that cells use to translate DNA sequences into protein is degenerate—there are more codons than amino acids, and different organisms use different proportions of the available codon options when synthesizing amino acid chains. If a gene comes from an organism that has codon usage differing from the organism where the gene is to be expressed, proteins may be poorly produced. This is often a problem when, as here, prokaryotic genes are to be expressed in eukaryotic systems. The correction is to modify the nucleotide sequence to use more-common codons throughout the sequence. At the same time, changes are often made to the nucleotide sequence to adjust the relative concentration of A/T and G/C base pairs to ratios more like those of the expression host.

Another complication is the targeting of proteins to the compartments of the eukaryotic cell to achieve the desired activity. DSG is active in the cyanobacterial thylakoid membrane, but its ability to insert into the endoplasmic reticulum, which we believe is optimal for its activity in modifying seed fatty acids, is unknown. DSG has domains that computer analysis indicates can span membranes, in a fashion similar to eukaryotic desaturases, so it is likely able to function as an integral membrane protein. For eukaryotic membrane proteins, two additional features of protein structure are widely recognized as important to their activity, an N-terminal endoplasmic reticulum signal sequence, and a C-terminal ER retention (and/or retrieval) signal. These are features of many, but not all, proteins that are active in the ER.

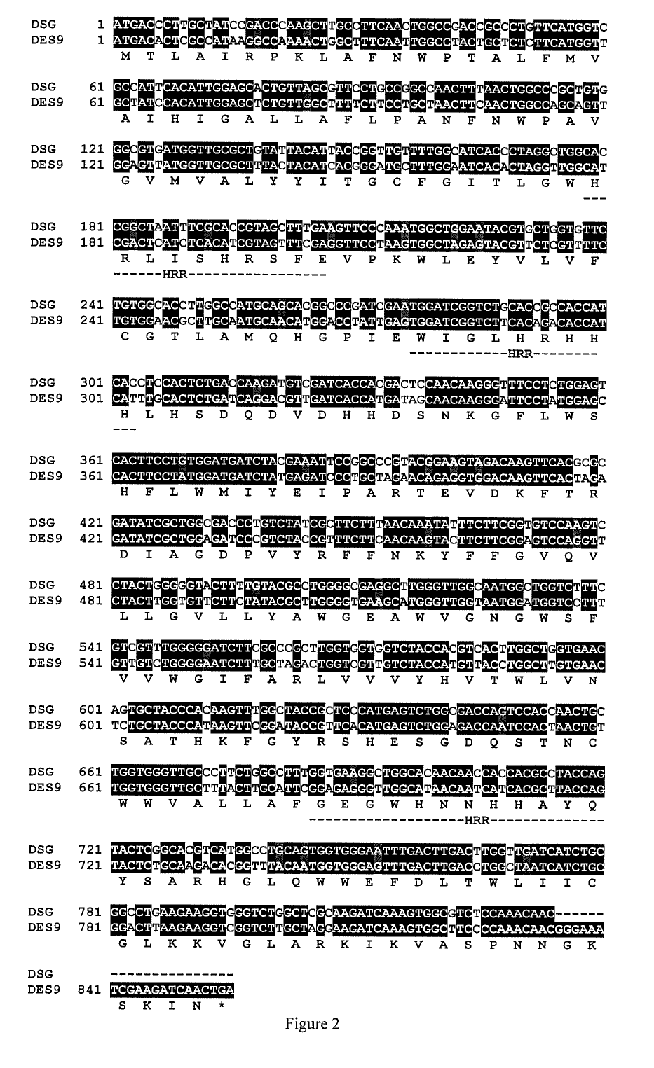

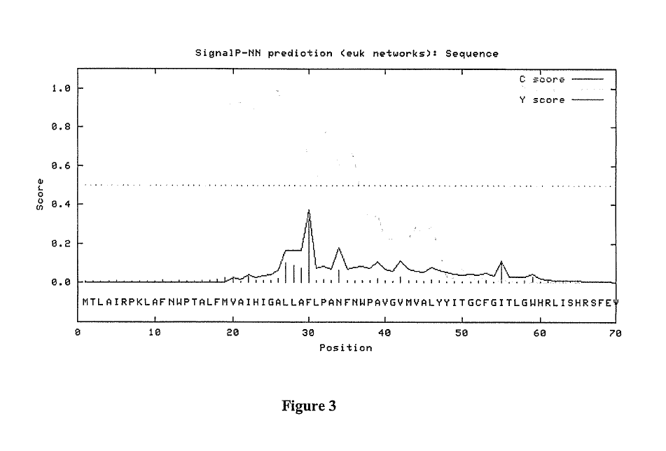

Computer sequence analysis (FIG. 2) indicated that DSG had N-terminal amino acid sequences that might be recognized by the eukaryotic host as an ER targeting signal, and many cyanobacterial membrane proteins use a signal-recognition pathway for membrane insertion that is similar to that found in the ER of eukaryotes. We therefore left the N-terminus of DSG unchanged. There was no indication of an C-terminal ER retention signal, so we modified the DSG open reading frame to encode one.

Apart from these coding and targeting issues, desaturase enzymes require electrons from an electron donor. In prokaryotes ferredoxin is the source of electrons, while eukaryotes rely on electron transfer from cytochrome b5.

A final obstacle to high activity from the DSG desaturase in eukaryotes is that, in Synechococcus the natural substrate for DSG is a fatty acid esterified to monogalactosyldiacylglycerol (MGDG). It is unclear how efficiently the enzyme might act on phosphatidylcholine (or other substrate) in the ER, although indirect evidence indicates that DSG is active in the ER in root cells of transgenic tobacco (Ishizaki-Nishizawa et al., 1996).

### DES9 Taken to the Next Step: Directed Evolution

In the face of these multiple obstacles to expression we attempted to span the gap between cyanobacterial and plant fatty acid desaturation by adopting a directed evolution strategy based on expressing large numbers of mutagenized proteins in the ole1 mutant of yeast, where we could detect increased activity, and afterward expressing the mutant proteins with highest activity in Arabidopsis seed.

Directed evolution is a powerful tool in protein engineering, especially when, as in the case of integral membrane desaturases like DSG, protein crystal structure is not available. Directed evolution accelerates the evolutionary process, making selection of desirable protein properties achievable in the laboratory. Using this strategy, we created multiple amino acid substitutions in DES9 which dramatically increase the desaturation activity in yeast. When we tested these mutagenized desaturase genes in plant seeds, the desaturases were much more active than their parent enzymes. The levels of 16:0 in transgenic seed expressing DES9* were reduced to about one-third the levels found in wild-type seed; when the coincident reduction of 18:0 levels by the desaturase was included in the analysis, total saturates are significantly reduced in comparison to the Arabidopsis wild type.

### Experimental Procedures

### Yeast Strain and Growth Media

In order to rapidly analyze the desaturase activity of DSG, DES9, and the changes we introduced in them, we used Saccharomyces cerevisiae strain ole1, which is disrupted in its sole desaturase, the Δ-9 desaturase OLE1. Yeast mutant in OLE1 require unsaturated fatty acid supplied in its media for growth (Stukey et al., 1989). Yeast strain DBY746 was used as the wild-type control for transformation and fatty acid analysis. Yeast were routinely cultured either in YPD (2% BACTO™ peptone, 1% yeast extract, 2% glucose) containing 0.5 mM linoleic acid (NuChek Prep, Elysian, Minn.) and 1% TERGITOL™, type NP-40 (Sigma), or in SD-ura media.

### Synthesis and Cloning of Des9.

The delta-9 glycerolipid desaturase DSG gene was amplified from Synechococcus elongatus PCC 6301, obtained from the American Type Culture Collection, using suitable primers designed to the 5-prime and 3-prime sequences of the open reading frame. After cloning the resulting fragments into PCR-Script® (Stratagene), a single clone whose sequence was identical to that for DSG in public databases was chosen for further analysis (SEQ ID NO: 2, see in FIG. 11B). A codon-optimized version of DSG (FIG. 12A, SEQ ID NO: 3) was commercially synthesized with addition of the protein sequence -GKSKIN (SEQ ID NO: 5) at its C-terminus, and designated Des9 (SEQ ID NO: 4, see FIG. 12B). Des9 was amplified using primers to incorporate appropriate restriction sites (Table 4). KOD hot start DNA polymerase (Novagen) was used for PCR amplification in all experiments unless otherwise indicated. The amplified fragment was restricted, then ligated into the 2μ-based yeast expression vector pMK195 (Overvoorde et al., 1996) under control of the constitutive ADH1 promoter, and after confirmation of the inserted sequence a pMK195-Des9 plasmid was chosen for further experiments.

TABLE 4
Primers used in this study.

| Primer name | Sequence (5′ to 3′) | Use |
|---|---|---|
| DEvo setup | TCTAGAGAATTCAAAAAAA | Clone Des9 into |
| primer | TGACACTCGCCATAAGGCC | PMK195 & |
| Des9 FOR | AAAAC | Template for |
| | (SEQ ID NO: 8) | First round |
| DEvo setup | CTCGAGGGATCCCTAATTA | mutagenesis |
| Des9 KSKIN | ATCAGTTGATCTTCGATTT | |
| REV Bam Xho | CCC (SEQ ID NO: 9) | |
| DEvo setup | TCTAGAGAATTCAAAAAAA | Clone DSG into |
| primer | TGACCCTTGCTATCCGACC | PMK195 |
| DSG FOR | CAAG | |
| | (SEQ ID NO: 10) | |
| DEvo setup DSG | CTCGAGGGATCCTCAACTA | |
| KSKIN REV Bam | ATTAATTGATTTTAGATTT | |
| Xho | TCC (SEQ ID NO: 11) | |
| Primer N | ATTTCAAGCTATACCAAGC | Generate |
| | ATACA | Mutagenesis |
| | (SEQ ID NO: 12) | PCR pools & |
| Primer C | CAACCTTGATTGGAGACTT | Yeast |
| | GACCAA | homologous |
| | (SEQ ID NO: 13) | recombination & |
| | | sequencing |
| | | primers |
| Q240N R | TAAACCGTGTCTTGCAGAG | Saturation |
| | TANNNGTAAGCGTGATGAT | mutagenesis & |
| | TG (SEQ ID NO: 14) | Combinatorial |
| Q240N F | TACTCTGCAAGACACGGTT | Saturation |
| | TACAAT | Mutagenesis |
| | (SEQ ID NO: 15) | |
| Q240P R | TAAACCGTGTCTTGCAGAG | Site |
| | TANGGGTAAGCGTGATGAT | mutagenesis |
| | TG (SEQ ID NO: 16) | |
| E69N R | GTACTCTAGCCACTTAGGA | Saturation |
| | ACNNNGAAACTACGATGTG | mutagenesis & |
| | AG (SEQ ID NO: 17) | Combinatorial |
| E69N F | GTTCCTAAGTGGCTAGAGT | Saturation |
| | ACGTTCTC | Mutagenesis |
| | (SEQ ID NO: 18) | |
| I129N R | GTCCACCTCTGTTCTAGCA | Saturation |
| | GGNNNCTCATAGATCAT | mutagenesis |
| | (SEQ ID NO: 19) | |
| I129N F | CCTGCTAGAACAGAGGTGG | |
| | ACAAG | |
| | (SEQ ID NO: 20) | |
| S213N R | ACAGTTAGTGGATTGGTCT | Saturation |
| | CCNNNCTCATGTGAACG | mutagenesis |
| | (SEQ ID NO: 21) | |
| S213N F | GGAGACCAATCCACTAACT | |
| | GTTGG | |
| | (SEQ ID NO: 22) | |
| I129N A131N R | CTTGTCCACCTCTGTTCTM | Saturation |
| | NNAGGMNNCTCATAGATCA | mutagenesis & |
| | T (SEQ ID NO: 23) | Combinatorial |
| I129N A131N F | AGAACAGAGGTGGACAAGT | Saturation |
| | TCACTAGAGAT | Mutagenesis |
| | (SEQ ID NO: 24) | |
| S213N G214N R | ACAGTTAGTGGATTGGTCM | Saturation |
| | NNMNNCTCATGTGAACG | mutagenesis & |
| | (SEQ ID NO: 25) | Combinatorial |
| S213N G214N F | GACCAATCCACTAACTGTT | Saturation |
| | GGTGGGTTG | Mutagenesis |
| | (SEQ ID NO: 26) | |
| E69R R | GTACTCTAGCCACTTAGGA | Site |
| | ACGCGGAAACTACGATGTG | mutagenesis |
| | AG (SEQ ID NO: 27) | |
| E69R F | GTTCCTAAGTGGCTAGAGT | |
| | ACGTTCTC | |
| | (SEQ ID NO: 28) | |
| Des9 Q240 | ACCGTGTCTTGCAGAGTAC | Q240R lock-in |
| lock R | TG (SEQ ID NO: 29) | experiment |
| Des9 Q240 | CAGTACTCTGCAAGACACG | |
| lock F | GT (SEQ ID NO: 30) | |
| PMK195 OLE1 | TCTAGAGAATTCAAAATGC | Clone OLE1 in |
| setup F | CAACTTCTGGAACTAC | PMK195 vector |
| | (SEQ ID NO: 31) | |
| PMK195 OLE1 | CTCGAGGGATCCTTAAAAG | |
| setup R | AACTTACCAGTTTCGTAG | |
| | (SEQ ID NO: 32) | |

### Random Point Mutagenesis and Transformation by Yeast Homologous Recombination

Mutagenized Des9 PCR pools were generated by using commercial mutagenesis kits, as described in the manufacturer's instructions. The primers used for mutagenesis were Primer N and Primer C (Table 4). All DNA products from PCR or restriction were purified using a commercial gel extraction kit.

For mutagenesis screening, yeast transformation was conducted by in vivo yeast homologous recombination. The preparation of competent cells and transformation procedures of LiAc method were performed according to Clontech Yeast Protocols Handbook with some modifications. In brief, mutagenized Des9 PCR products were transformed into ole1 competent cells with linearized pMK195-Des9 vector from which Des9 coding sequence had been entirely removed by restriction with EcoRI and Ban/HI, leaving homology to the vector only at the 5′ and 3′ ends. The molar ratio of the vector and insert DNA were 1:3 for the transformation. The transformed yeast cells were plated onto SD-Ura medium (complete minimal medium containing 1% TERGITOL™, but lacking uracil) without fatty acid supplement. Candidate colonies which appeared earlier than those from a DES9 control transformation were chosen for further analysis. Candidate colonies were streaked on SD-Ura without supplemental fatty acids at 30° C., and their growth within 48 h indicated that expression of the mutagenized DES9 variants (DES9*) rescued the ole1 unsaturated fatty acid auxotrophy.

### Yeast Fatty Acid Analysis

Single yeast colonies from streak plates were inoculated into SD-Ura medium lacking supplemental fatty acids. After for 1-3 d, the cells from 1 ml of culture were collected by centrifugation and washed once with water, except yeast cultured with exogenous fatty acids (0.5 mM, 1% tergitol) were collected and the pellets washed once with 1% tergitol, then twice with water. Fatty acid methyl esters (FAMEs) were prepared by re-suspending the pellets in 1 ml of 2.5% sulfuric acid in methanol, followed by incubation at 80° C. for 1 h. The FAME derivatives were extracted into hexane and analyzed by gas chromatography with quantitation by flame ionization detection. Chromatography parameters were 210° C. for 2 min followed by a ramp to 245° C. at 10° C. per min with a 4 min final temperature hold. For some fatty acid analysis experiments, we compared the fatty acid profiles of DES9 constructs with an otherwise identical construct which expresses the wild-type OLE1 gene.

### Mutation Identification and Sequence Analysis

Candidate yeast clones were isolated from yeast using a commercial yeast plasmid miniprep kit. DES9* were amplified by PCR using Primer N and Primer C (Table 4), and the PCR products purified before sequencing and sequence analysis.

### Amino Acid Changes Independent of Q240

To probe for mutations at locations other than the recurring changes at Q240, random mutations were introduced in two separate PCR amplifications using the GeneMorph II Kit. The 5-prime end of the open reading frame was amplified with Primer N plus primer “Q240 lock R”, and the 3-prime end by “Q240 lock F” plus Primer C. Finally, the two ends were joined by overlap extension PCR using Primer N and Primer C, with a mixture of the two previous reactions as template. Yeast transformation, selection of candidate clones, and analysis were as described above.

### Saturation Mutagenesis at Selected Codons

Because successful mutations occurred often at codons for amino acids E69, 1129, S213 and Q240 at high frequency, a saturation mutagenesis was performed separately at each codon for these amino acids by replacing the target codon with NNN, using overlap-extension PCR and pMK195-Des9 plasmid as template. For example, saturating mutations were introduced at the codon for amino acid 69 by initially conducting two separate amplification reactions using Primer A with Primer E69NR in one reaction and Primer E69NF with Primer C (Table 4). Agarose gel-purified reaction products were combined and amplified together with Primer N and Primer C. The resulting products were co-transformed with linearized pMK195-Des9 vector into ole1, and colony analysis, fatty acid analysis and DNA sequence analysis was as above. Saturation mutagenesis at codon 129, 213 and 240 followed the same procedures with the appropriate primers (Table 4).

### Combinatorial Saturation Mutagenesis

Combinatorial saturation mutagenesis was performed in a similar fashion to the saturation mutagenesis procedure, except that the PCR protocol was designed to simultaneously test all possible codons, and therefore all 20 possible amino acid variations, at six positions identified above as important to desaturase activity. Accordingly, overlap extension PCR was used to construct a library all possibilities were incorporated at six positions (69, 129, 131, 213, 214, and 240) of DES9 simultaneously. Five independent PCR amplification reactions of Des9 were used to generate partial DNA fragments overlapping each other: Primer N with Primer E69NR, Primer E69NF with Primer I129N A131NR, Primer I129N A131NF with Primer S213N G214NR, Primer S213N G214NF with Primer Q240NR and finally Primer Q240NF with Primer C. The resulting five DNA fragments were agarose gel-purified and used as template for PCR amplification using Primer N and Primer C. Yeast transformation, screening and analysis were as described above.

### Random Mutagenesis by DNA Shuffling

Multiple DES9* identified from random mutagenesis and saturation mutagenesis including K8R, E69K/G/R, I129T/P, A131V, S213P/Y/R, R132K, G214R and Q240R/K, were shuffled to create mutagenesis DNA pools using a Jena Bioscience DNA-Shuffling Kit (Jena Biosciences). The DNA pools were then used for yeast transformation and analysis as described above.

### Topology Prediction

Topology predictions for the DES9 are based on the TMHMM version 2.0 algorithms (website located at cbs.dtu.dk/services/TMHMM, (Krogh et al., 2001)). The model was in close agreement with predicted and validated topologies of other Δ9 glycerolipid desaturases.

### Plant Transformation

The amplified fragments of DSG-KSKIN, Des9, DSG-Q240R and Des9* were cloned into the pENTR™-D-TOPO® vector (Invitrogen) and recombined into vector pGate-DsRed-Phas vector (Lu et al., 2006) with DsRed as a marker (Stuitje et al., 2003) using commercial GATEWAY™ reagents. Following transformation of these DES9 and DES9*-expressing constructs into Agrobacterium tumefaciens strain GV3101, Arabidopsis thaliana ecotype Columbia plants grown in chambers under continuous fluorescent light (100-200 μmol m⁻² s⁻¹) at 22° C. were transformed using an established floral dip method (Clough and Bent, 1998).

### Seed Fatty Acid Analysis

For Arabidopsis, 5 transformed red seeds were incubated in 1 mL of 2.5% (v/v) sulphuric acid in methanol for 1.5 h at 80° C. (Miguel and Browse, 1992). The resulting fatty acid methyl esters were extracted into hexane and analyzed by gas chromatography and identified by flame ionization detection. Chromatography parameters were 210° C. for 2 min followed by a ramp to 245° C. at 10° C. per min and a 4 min final temperature hold.

### Results

### Engineering an Active DES9 for Eukaryotes

Our previous studies have shown that native DES9 desaturase activity is too low to substantially reduce 16:0 in seeds. In addition, when a DSG cDNA was cloned under control of a constitutive ADH1 promoter into pMK195 and expressed it in ole1Δ mutant yeast, it did not readily complement the requirement of this strain for unsaturated fatty acids. To increase the desaturase activity of DSG, we synthesized a DSG open reading frame with codons optimized for eukaryotic expression, to improve the efficiency of protein synthesis (FIG. 2 and Table 5). When we synthesized the open reading frame and adjusted the ratio between AT and GC pairs, we also extended the coding region with nucleotides to encode an endoplasmic-reticulum (ER) retention signal motif. The purpose of the -GKSKIN (SEQ ID NO: 5) signal at the C-terminus was to improve subcellular localization. This modified DNA sequence, termed Des9, was cloned into pMK195.

The characteristics of DSG and Des9 are as follows:

| | |
|---|---|
| Freq | % |

DSG: Length: 834

| | | |
|---|---|---|
| A: | 155 | 18.6% |
| C: | 239 | 28.7% |
| G: | 224 | 26.9% |
| T: | 216 | 25.9% |
| GC: | 463 | 55.5% |

Des9 Length: 855

| | | |
|---|---|---|
| A: | 198 | 23.2% |
| C: | 195 | 22.8% |
| G: | 208 | 24.3% |
| T: | 254 | 29.7% |
| GC | 403 | 47.1% |

TABLE 5
DSG and Des9 Codon usage in yeast and Arabidopsis; Analyzed
by online tool Genscript (website located at genscript.com)

| | | | Frequency of low |
|---|---|---|---|
| | Codon Adaptation | | frequency (<30%) |
| | Index (CAI)^{a} | GC content^{b} | codons^{c} |
| DSG in yeast | 0.61 | 55.63% | 7% |
| Des9 in yeast | 0.7 | 47.22% | 2% |
| DSG in Arabidopsis | 0.67 | 55.63% | 3% |
| Des9 in Arabidopsis | 0.83 | 47.22% | 0% |

^{a}Codon Adaptation Index (CAI): CAI of 1.0 is considered to be ideal while a CAI of >0.8 is good.
^{b}Ideal percentage range of GC content 30% to 70%.
^{c}The percentage distribution of codons in computed codon quality groups.

Complementing the ole1 Mutant

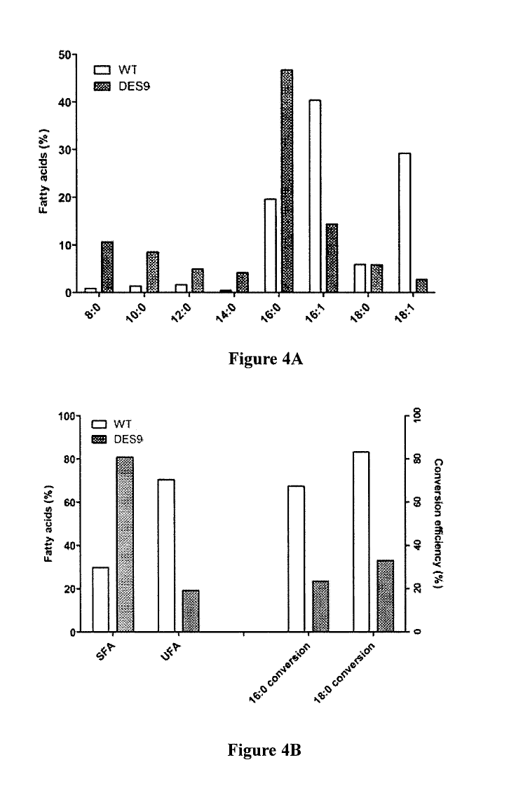

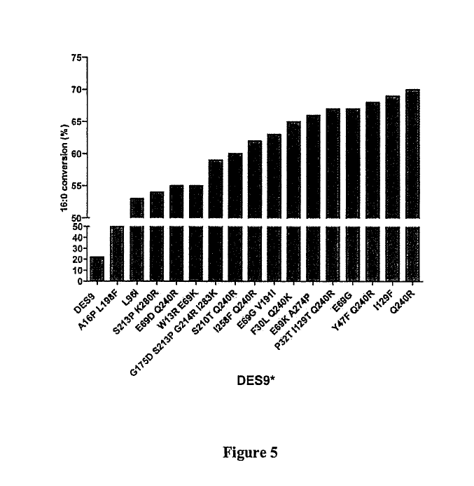

When we transformed the ole1Δ yeast strain with the pMK195-Des9 construct, we found that DES9 expression could barely complement the fatty acid auxotrophy of the mutant; colonies appeared on solid SD-ura media without fatty acid supplement a full 6 d after transformation. The growth of ole1 transformed with the DES9 expression construct was very poor in liquid culture; the cells grew slowly and densely clumped. We analyzed the fatty acid content of these cells by preparing fatty acid methyl esters followed by gas chromatography. The fatty acids of wild-type yeast have two principal unsaturated fatty acids, 16:1Δ9 and 18:1Δ9, which make up 40% and 30%, respectively, of the total fatty acid composition, produced by the OLE1 desaturase from the less-abundant saturated 16:0 and 18:0 fatty acids (FIG. 4A). Wild-type yeast also have small proportions of saturated fatty acids with carbon-chain lengths shorter than 16C, including 8:0, 10:0, 12:0 and 14:0. Yeast expressing DES9 had both 16:1Δ9 and 18:1Δ9 fatty acids, but at much lower proportions than wild type, only 15% and 5%, respectively (FIG. 4A). The saturated fatty acid content of the DES9-expressing yeast is three times higher than wild-type yeast; 80% of the fatty acids are saturated, compared to only 23% saturated fatty acids in wild type. The distribution of these fatty acids is interesting: there are high levels of 16:0, but very little 18:0, and much higher proportion of saturated fatty acids with hydrocarbon chains shorter than 16C. When the activity of DES9 is considered as conversion of 16:0 and 18:0 to their respective monounsaturates, conversion was only 23% and 33% respectively, very low compared to 67% and 83% for wild-type yeast (FIG. 4B).

### Random Mutagenesis of Des9 by Error Prone DNA Polymerase

Because the ole1Δ yeast strain requires C16 or C18 unsaturated fatty acid to grow and DES9 expression only poorly complemented the mutant phenotype, we sought to create and identify mutants with improved Δ9-desaturation activity by directed evolution, screening for mutants that allow ole1Δ to rapidly form colonies on solid medium.

We subjected Des9 to random mutagenesis using a Gene Morph II Random Mutagenesis Kit, which relies on an error-prone thermostable polymerase to induce mutations in the target nucleotide sequence. We amplified the Des9 sequence with the error-prone polymerase, using long primers that incorporated flanking sequences of about 40 base-pairs of homology to pMK195. We simultaneously co-transformed competent ole1Δ cells with both the PCR products and the restriction-digested vector, relying on in vivo yeast recombination to introduce mutagenized Des9 fragments into the vector. Colonies arose as early as 48 hrs after transformation on media selecting for the URA3+ vector marker on plates without fatty acid supplement. Fast-growing colonies were streaked on a second unsupplemented selection plate, and analyzed after 2 d growth by inoculation into SD-ura liquid medium. After three days, these cultures were harvested, and fatty acid methyl esters (FAMEs) were prepared from the cells, followed by analysis by gas chromatography (GC). GC results revealed 22 cultures with obvious increases in 16:0 conversion compared to the DES9 control. While DES9 converted only 22% of the available 16:0 substrate to 16:1 product, conversion by mutant forms ranged from 50 to 70% (FIG. 5). To distinguish mutant DES9 derivatives with increased desaturase activity, we refer to them as DES9*. When the DES9* open reading frame was amplified by PCR from yeast clones with increased activity, DNA sequence analysis of the PCR product revealed between 1 and 6 nucleotide changes per insert, in the range of the design for the protocol used. Expression of DES9* with single mutations at four different amino acid residues E69G, L961, I129F and Q240R each increased conversion of the 16:0 substrate to 16:1 Δ9 by 2.4-3.2 fold (FIG. 5). Changes at positions E69, I129, S213 and Q240 were identified in multiple clones (Table 6). Remarkably, changes at the DES9 residue Q240 were found thirteen times, a much higher frequency than any other change; changes at Q240 with higher desaturation activity were found both singly and combined with other mutations (Table 6). Interestingly, among the thirteen changes observed at Q240, seven mutants with the highest conversion of 16:0, averaging 66%, have the single mutation Q240R (Table 6). The second most common residue to be changed is E69; five mutants with either E69 alone changed, or combined with other changes, produce 63-69% 16:0 conversion.

TABLE 6
Comparison of frequency of random mutagenesis and saturation
mutagenesis at four sites of DES9 and conversion of 16:0.

| | Substitu- | | Most | | |
|---|---|---|---|---|---|
| | tion | | superior | | |
| | from 1^{st} | | mutations | | Highest |
| | random | Times/22 | from | Times/total | conver- |
| | muta- | sequenced | saturation | sequenced | sion of |
| Position | genesis | clones | mutagenesis | clones | 16:0 |
| Q240 | R/K | 12/1 | R | 8/14 | 71% |
| E69 | G/K/D | 2/1/1 | R | 5/11 | 69% |
| I129 | F/T | 1/1 | T | 2/8 | 63% |
| S213 | P | 2 | R | 6/8 | 66% |
| DES9 | — | — | — | — | 32% |
| (control) | | | | | |

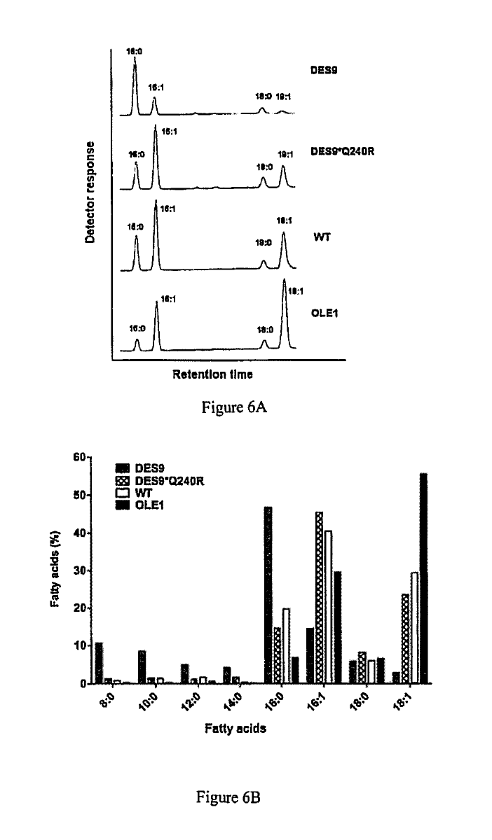

When we analyzed the fatty acid profile of yeast expressing the DES9*Q240R protein after 48 h growth, several changes were observed. At 48 h after culture inoculation, DES9-expressing yeast had 46% 16:0, while expression of DES9*Q240R reduced 16:0 to 16% (FIG. 6B). The level of 16:1 in DES9*Q240R-expressing yeast was more than double the 16:1 measured in DES9-expressing yeast, and the level of 18:1 measured in was five-fold higher than seen with DES9, although not as high as seen with overexpression of the native yeast OLE1 desaturase (FIG. 6). It is also notable that yeast expressing the DES9 parent sequence had significant saturated fatty acids with chain lengths of fewer than 16 carbons, amounting to about 28% of the total fatty acids. These saturates were found at much lower levels in the wild-type and DES9*Q240R strains (FIG. 6). The fatty acid analyses confirm that the DES9*Q240R variant was much more active than DES9.

### Random Mutagenesis of Des9 Using Deoxyribonucleotide Analogs

Since all methods of mutagenesis exhibit some bias in their products, we performed a second random mutagenesis based on amplification that includes deoxyribonucleotide analogs, using the same cloning and selection procedures as before. Twelve colonies, that when grown and analyzed by GC produced more than 50% conversion of 16:0, were chosen for sequence analysis of their amino-acid changes. The sequencing results revealed that two of the best sequenced clones included Q240R, simultaneously with changes to other amino acid codons, producing yeast cultures with 70% and 72% conversion of 16:0 to 16:1. In addition, a single mutation of A131V, close to the previously detected 1129 mutation, also increased 16:0 conversion to 55%. Taken together, the results from two different methods of inducing mutations further confirmed that Q240R is a most critical amino acid change to improve DES9 activity.

### Saturation Mutagenesis at Selected Codons

Amino acid changes at Q240, E69, I129 and S213 of DES9 were obtained multiple times in our original screen (Table 6), indicating that changes in these residues are especially useful for improving desaturase activity in yeast. We separately mutagenized each of these residues with completely degenerate primers to test every possible amino acid substitution at each site. After selection, fatty acid analysis, and DNA sequence analysis, many mutant forms with the highest conversion of 16:0 were identical to mutations already detected in our original experiments. Of fourteen clones with changes at the Q240 locus; eight were Q240R and one Q240K. Eleven sequenced clones were changed at the E69 locus; five were E69R and one E69K (Table 6). Of the eight sequenced variants at the 5213 locus, six were S213R. Although I129 seemed more plastic and had a range of changes, two I129T clones were identified among 8 sequenced clones. None of the other single amino acid changes observed at these codons had desaturase activity greater than, or even equivalent to, the most frequently detected mutations. These analyses confirmed Q240R as the evolved protein providing the highest conversion of 16:0 to 16:1; E69R provided the second highest conversion.

### Test Amino Acid Changes Independent of Q240R

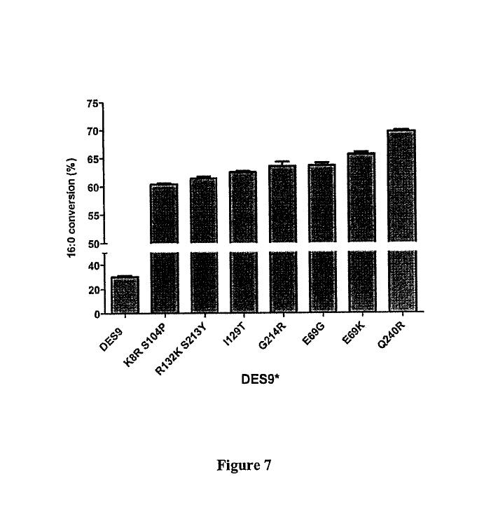

The single amino acid change Q240R was discovered with high frequency (Table 6) and produced high desaturation of 16:0 and robust yeast growth. To explore other critical amino acid changes for improving DES9 activity, we mutagenized Des9 with a PCR method specifically designed to retain the native Q240 residue with the six amino acids surrounding it, while examining changes throughout the rest of the coding sequence. When yeast were transformed and screened as before, 16 colonies with rapid growth on selective plates were chosen for GC analysis: seven of them, with 60-66% conversion of 16:0, were changes at E69, I129 and S213, including E69K, E69G, and I129T, identical amino acid changes to those found in the previous experiment. We did find three coding sequences with mutations at new locations in the protein, including a single change of G214R and two mutant forms with simultaneous changes at two locations, K8R/S104P and R132K/S213Y. However, no mutant form of the protein was found that desaturated 16:0 more successfully than DES9*Q240R (FIG. 7). These results confirmed our previous findings, and indicated that we did not overlook the best mutations in the random mutagenesis experiment.

### Combinatorial Saturation Mutagenesis and Gene Shuffling

Using the information revealed by random mutagenesis experiments, we tested whether some combination of mutations at six identified critical sites, E69, I129, A131, S213, G214 and Q240, would produce a sequence coding for a more active desaturase. Using primers randomized at the appropriate locations (Table 4), we used overlap extension PCR to create a DNA pool that would represent every possible combination of the changes at each of these residues. We transformed ole1 as before, followed by selection and analysis. Twenty-five fast-growing colonies were cultured in liquid medium and subjected to GC analysis at 24 and 48 h after culture inoculation. For two clones, GC analysis indicated 16:0 conversion in excess of 80%, higher than the activity of DES9*Q240R at both time points (Table 7). When sequenced, these clones were found to contain multiple amino acid changes. For brevity, the two best variants are referred to as DES9*15 (E69R/I129A/G214R) and DES9*12 (E69A/I129C/A131V/S213P/G214P).

We used a PCR strategy to shuffle our most active amino acid changes together, creating two DNA pools coding for Des9 with mutations K8R, E69K/G/R, I129T/P, A131V, S213P/Y/R, R132K, G214R, all either in combination with Q240R, Q240K, or in combination with the original Q240 residue. Sixteen colonies resulting from transformation with the first DNA pool and 8 colonies from the second pool were provided rapid growth on the selection plates and were chosen for GC analysis. After analysis of liquid cultures, only two yeast strains, with conversion of 16:0 higher than DES9*Q240R, were analyzed further. Both these strains carried a R132K mutation, DES9*23 (R132K/G214R/Q240R) and DES9*24 (K8R/H88Q/R132K/Q240K) (Table 7). When yeast cultures expressing these DES9* were assayed 48 h after inoculation each produced 82% conversion of 16:0 to 16:1. These mutant lines are the most active desaturases obtained from our directed evolution experiments. Their activities represent a greater than three-fold increase in the conversion of 16:0 to 16:1 relative to the DES9 parent.

TABLE 7
Comparison of best mutants selected from combinatorial saturation and shuffling mutagenesis
by high copy vector pMK195. (Data are means of three replicates, ± S.D.)

| | | 16:0 conversion (%) | | 18:0 conversion (%) | |
|---|---|---|---|---|---|
| Strains | Mutations | 24 h | 48 h | 24 h | 48 h |
| DES9 | None | 22.3 ± 1.0 | 26.3 ± 3.9 | 29.7 ± 5.9 | 35.0 ± 6.1 |
| Wild type | None | 67.8 ± 0.7 | 71.9 ± 1.7 | 83.4 ± 0.3 | 86.4 ± 0.8 |
| OLE1 | None | 78.2 ± 0.9 | 84.2 ± 2.0 | 88.8 ± 0.7 | 92.1 ± 0.5 |
| DES9*Q240R | Q240R | 54.7 ± 1.6 | 73.6 ± 2.5 | 51.1 ± 9.2 | 72.7 ± 3.6 |
| DES9*(E69R/Q240R) | E69R, 240R | 57.9 ± 7.9 | 73.8 ± 4.2 | 63.4 ± 6.0 | 72.0 ± 4.2 |
| DES9*15 | E69R, I129A, G214R | 66.0 ± 5.2 | 77.3 ± 2.9 | 70.2 ± 8.5 | 82.1 ± 1.2 |
| DES9*12 | E69A, I129C, A131V, | 69.3 ± 1.0 | 78.7 ± 1.9 | 74.0 ± 5.5 | 83.1 ± 2.7 |
| | S213P, G214P | | | | |
| DES9*23 | R132K, G214R, Q240R | 65.1 ± 0.1 | 82.0 ± 1.9 | 69.8 ± 5.1 | 81.1 ± 0.2 |
| DES9*24 | K8R, H88Q, R132K, | 67.9 ± 1.7 | 82.3 ± 2.0 | 68.0 ± 1.9 | 81.5 ± 2.7 |
| | Q240K | | | | |

Arabidopsis Seeds Expressing Des9* have Reduced 16:0 and 18:0, and Higher Levels of 16:1 and 18:1

To test the desaturation activity of mutagenized DES9* in plant seeds, we separately transformed Arabidopsis with constructs expressing DES9, DES9*Q240R or DES9*(E69R/Q240R) under the control of a seed-specific phaseolin promoter, using a vector expressing the DsRed screening marker. For comparison, we also used identical vector constructs to express both DSG-KSKIN, the original cyanobacterial protein except for addition of the -KSKIN (SEQ ID NO: 5) ER retention sequence, and DSG-Q240R, the DSG-KSKIN sequence with the single amino acid change at Q240. As preliminary test we picked five T1 red seeds from each transformation and analyzed their fatty acids by GC, comparing them to brown, untransformed seed. While DES9 only reduced 16:0 from 8.4 to 7.2%, DES9*Q240R and DES9*(E69R/Q240R) both reduced 16:0 to about 4% of the total fatty acids.

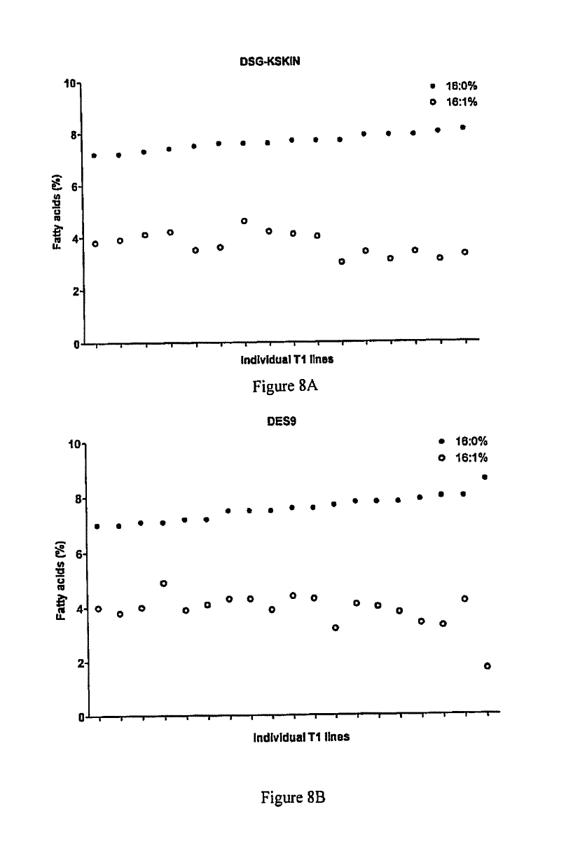

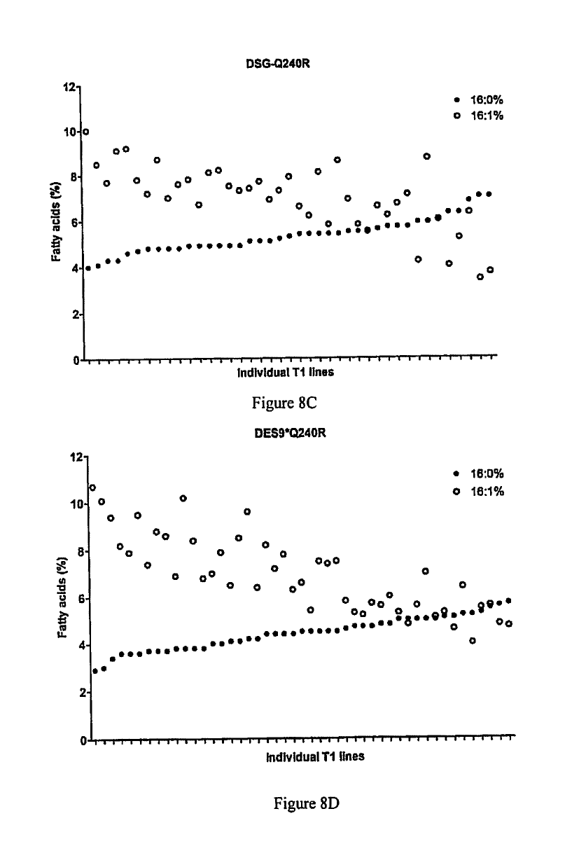

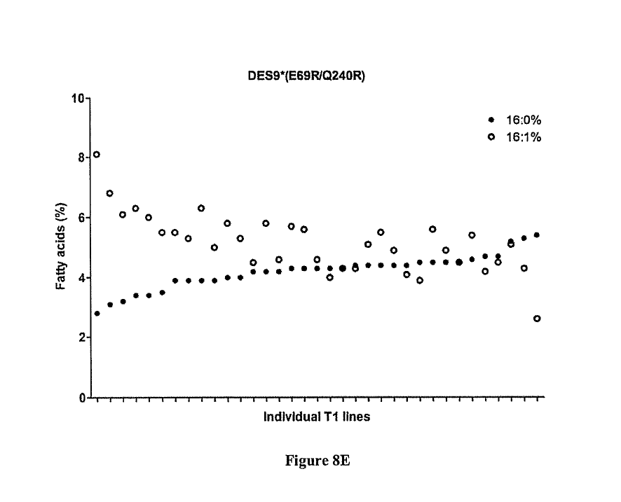

Red, transformed seed were sown in pots and the seed harvested at maturity. When the T2 seeds from multiple T1 plants were analyzed, expression of the DES9 and DES9* constructs each produced a range of desaturase activities, as expected for transgenic plants (FIG. 8). The range of activities clearly confirm that expression of DES9* constructs have greater activities than DES9. When the averages of many T2 lines are compared (Table 8) the increased activity of the DES9* constructs is evident. While levels of 16:0 on average in DSG-KSKIN and DES9 are 7.6%, compared to the 9% in wild type seed, DES9*Q240R and DES9*(E69R/Q240R) have much lower 16:0, averaging about 4.3%. The construct DSG-Q240R, which has both the -KSKIN retention signal and introduced mutation Q240R, exhibited desaturase activity intermediate between DSG-KSKIN and the DES9* constructs (FIG. 8).

TABLE 8
Levels of 16:0, 16:1 and 16:0 conversion in T2 bulk
seeds; n, total number of T1 plants for T2 bulk
seeds analysis. Data represent means, ± S.D.

| | | | 16:0 |
|---|---|---|---|
| T2 bulk seeds | 16:0% | 16:1% | conversion (%) |
| Wild Type | 9.1 ± 0.1 | 0.4 | 4.6 ± 0.2 |
| DSG-KSKIN (n = 16) | 7.6 ± 0.3 | 3.7 ± 0.5 | 32.6 ± 3.3 |
| DES9 (n = 19) | 7.6 ± 0.4 | 3.9 ± 0.6 | 33.7 ± 4.9 |
| DES9*Q240R (n = 47) | 4.4 ± 0.7 | 6.9 ± 1.7 | 60.3 ± 8.9 |
| DES9*(E69R/Q240R) (n = 35) | 4.2 ± 0.6 | 5.1 ± 1.0 | 54.8 ± 7.7 |
| DSG-Q240R (n = 41) | 5.3 ± 0.7 | 7.0 ± 1.5 | 56.4 ± 8.6 |

The lowest levels of 16:0 in both DES9*Q240R and DES9*(E69R/Q240R) transgenic lines were just below 3% (FIG. 8). Since the average of low 16:0 lines in DES9*Q240R and DES9*(E69R/Q240R) were similar, we chose two lines expressing DES9*Q240R with the lowest level of 16:0 whose genetic segregation indicated that the T-DNA insertion was at a single locus, for further study. The levels of 16:0 in this T3 homozygous line is only 2.7%, less than half that measured for DES9 and DSG-KSKIN, which are 6.4% and 6.6% respectively (Table 9): this reduction is a greater that 65% decrease in 16:0 relative to wild type controls. In these Arabidopsis homozygous T3 seeds, the 18:0 levels are also clearly reduced (Table 9). The lowest level of 18:0 seen here is 1.5% for the DES9*Q240R seeds. The proportion of both 16:1 and 18:1 fatty acids increases as the 16:0 and 18:0 decrease; 16:1 is highest in the DES9*Q240R line, representing more than 10% of the total fatty acids, a 20-fold increase over the wild-type level of 16:1. The increase in 18:1 reaches 20% of the total, a full ⅓ increase over the wild-type level (Table 9). Saturated 16:0 and 18:0 make up 11.1% of the total fatty acids in wild-type seed, but only add up to 4.2% in DES9*Q240R seeds, a reduction of 62.2%. Further reductions in saturated fatty acids and increases in 16:1 and 18:1 levels in Arabidopsis seeds are expected in these types of lines.

TABLE 9
Changes in 16C and 18C levels in DSG-KSKIN, DES9,
and DES9*Q240R T3 homozygous lines. Data represent
lines with lowest levels of 16:0 so far examined.

| | | | | | Reduction in |
|---|---|---|---|---|---|
| | | | | | saturates (% of |
| T3 seeds | 16:0% | 16:1% | 18:0% | 18:1% | wild-type) |
| Wild Type | 8.5 | 0.5 | 2.6 | 14.9 | 0 |
| DES9 | 6.4 | 3.8 | 2.2 | 16.3 | 22.5 |
| DSG-KSKIN | 6.6 | 3.6 | 2.1 | 15.7 | 21.6 |
| DES9*Q240R | 2.7 | 10.1 | 1.5 | 20.1 | 62.2 |

### Discussion

### Obstacles to Expression of Prokaryotic Desaturases

To achieve successful expression of a prokaryotic desaturase in both yeast and plants, we addressed several issues that might block activity of the DSG glycerolipid 16:0 desaturase. Since there are often sharp differences between the codons used by prokaryotes and eukaryotes, which can dramatically reduce the production of protein when a prokaryotic gene is expressed in eukaryotes, we synthesized an alternative DNA sequence, Des9. The nucleotide sequence and codon usage of Des9 is more likely to produce high protein levels in eukaryotes (FIG. 2 and Table 5). Since the localization of proteins can also have significant effect on their activity, we also added nucleotides to encode a C-terminal eukaryotic ER retention signal to this new Des9, based on the ER targeting signal of the FAD3 desaturase of Arabidopsis (McCartney et al., 2004).

DSG is an glycerolipid Δ9-desaturase of Synechococcus elongates PCC 6301. As a prokaryotic Δ9-desaturase, DSG uses ferredoxin as electron donor for desaturation, introducing a cis-double bond at the Δ⁹ position of both 16- and 18-carbon saturated fatty acids linked to membrane lipids. DSG has been shown to be an active 16:0 desaturase, with somewhat less activity on its 18:0 substrate, when the enzyme is expressed in E. coli. When expressed in yeast, our DES9 protein was very active on both 16:0 and 18:0 (Table 7).

### Directed Evolution Increased Desaturase Activity

We were very successful in finding altered protein sequences in our first evolutionary trials using an error-prone polymerase. Fast-growing colonies were easy to identify and the desaturase activity of several of them indicated doubled or trebled desaturase activity (FIG. 5). On further analysis, these high-activity yeast were expressing variant DES9* with a few or sometimes single amino acid changes. When we used a different method based on deoxynucleotide analogues, we recovered some variant protein sequences identical to those found by the first method. Since DES9* which included a Q240R or Q240K were found very frequently, we conducted an experiment designed to retain the original Q240 residue but detect other changes that would increase the desaturase activity. Most of the changes that we detected in this way had already been discovered by the single mutation approaches, but we did identify a few new amino acid variants that provided increased desaturase activity. We used this collection of information about which residue changes were conducive to producing higher desaturase activity to design more experiments. First, using a PCR-based strategy, we tested every possible amino acid at each of these identified, significant loci. The results showed that we had already identified variants that were as good as any others. The second experiment shuffled together all the likely variants that we had so far identified, to see if some combination of these changes would give increased activity. Indeed, two variants from this experiment, DES9*23 and DES9*24, produced the highest level of desaturation detected in all our experiments: ole1 yeast expressing these DES9* converted more than 80% of 16-carbon fatty acid to 16:1 (Table 7).

### Residues Adjacent to Histidine Boxes are Critical for Improving DSG Activity

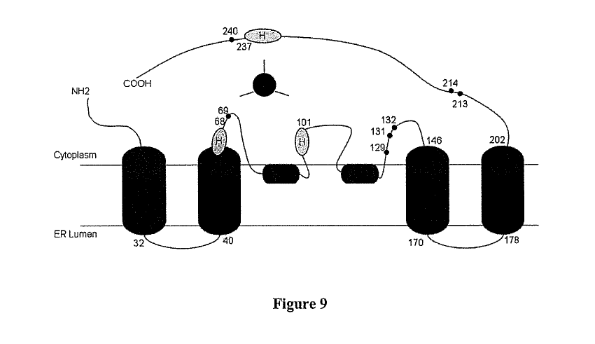

DSG has three histidine-rich boxes essential for the activity of membrane-bound fatty acid desaturases. In the DSG sequence, these lie at locations 60-69 (HRLISHRSFE, SEQ ID NO: 32), 93-101, (WIGLHRHHH, SEQ ID NO: 33); and 229-240 (GEGWHNNHHAYQ, SEQ ID NO: 34). Interestingly, two of the changes which were discovered most often, and which had the greatest effects in increasing desaturase activity are E69 and Q240, are proximate to the first and third histidine boxes, respectively (FIG. 9).

Because the iron ions coordinated by these histidine clusters are critical to the electron transfer reactions of desaturation, it is tempting to speculate that the interactions of the most active DES9* with the eukaryotic electron transfer system have been improved. Whether this is the case, or whether the DES9* desaturases interact more effectively with the substrate, it is clear that the changes extend from the yeast model system to Arabidopsis. Directed evolution of other enzymes has demonstrated that residues close to active sites are more important than distant ones for improving enzyme activity or substrate specificity. Our random mutagenesis and targeted mutagenesis results demonstrate that a single amino acid change at Q240 or E69, for example, can significantly improve desaturase activity: expression of DES9*Q240R, DES9*I129F and DES9*E69G improve 16:0 conversion more than two-fold over the DES9 parent in yeast (Table 7); those tested are also active in Arabidopsis seed (Table 9).

### Histidine-Rich Boxes and Positively Charged Residues

Much of the improved DES9 activity we saw was due to amino acids changes, either singly or in combination, which were exposed to the cytosol based on a predicted topological model of the DES9 desaturase (FIG. 9), notably the E69 and Q240 residues near the first and third histidine boxes which outline the active site of the enzyme (FIG. 9). Residues in the vicinity of the histidine boxes are known to influence the catalytic reaction outcome of desaturases. Mutations that create substitutions for any of the conserved histidine residues eliminate desaturase enzyme activity, demonstrating the important role for desaturases binding ferric iron at these sites. Improved DES9* activity is often accompanied by mutations that incorporate amino acids with positive charges like E69R and Q240R; these changes in charge may increase the strength of binding of the ferric iron.

An advantage of the directed evolution approach is that it can operate in the absence of a known protein crystal structure or full understanding of the mechanism of the activity under selection. The increased activity of DES9 may be due to more felicitous interactions with the eukaryotic cytochrome b5 electron transport chain, as suggested by the dramatic increase of activity for selected mutations near the histidine-rich regions which are important to electron transfer mediated by the associated iron molecules (FIG. 9). It is also possible that the observed mutations increase the affinity of the desaturase for the lipid, or lipids, that constitute its substrate both in yeast and in plants, or that the mutations enhance the stability of the expressed protein, since desaturases are often considered to be short-lived proteins.

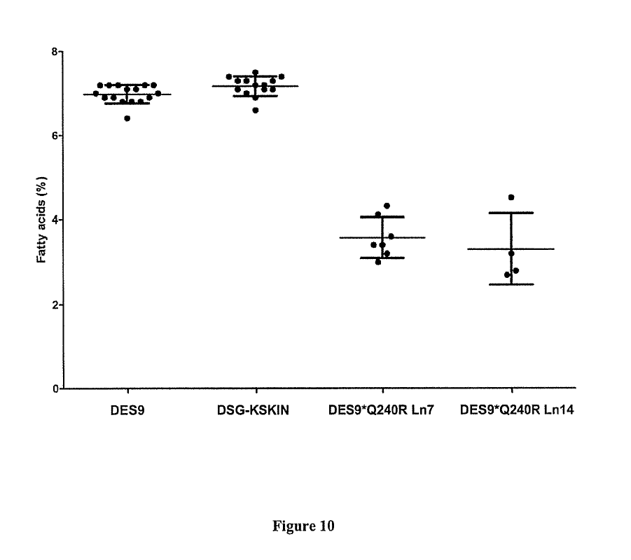

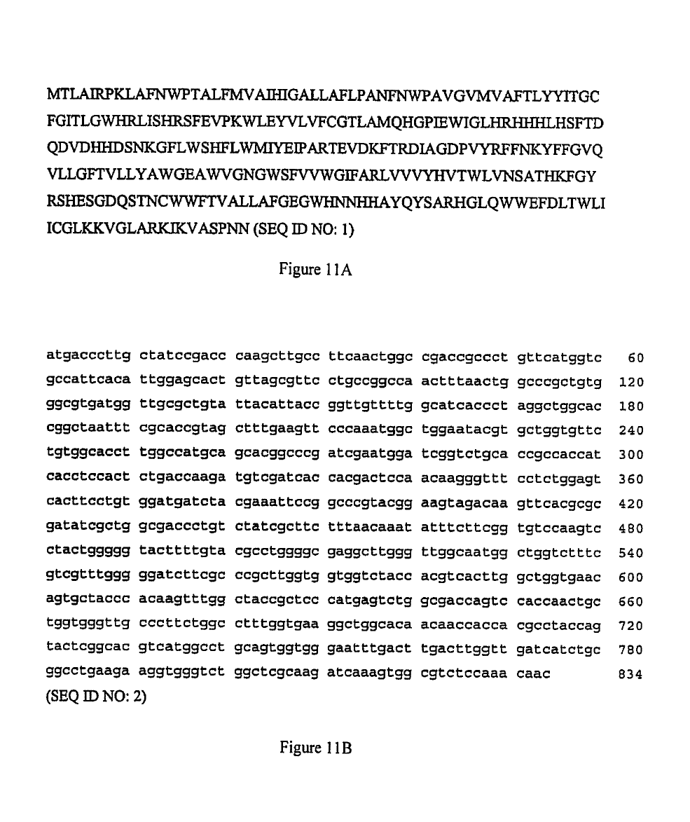

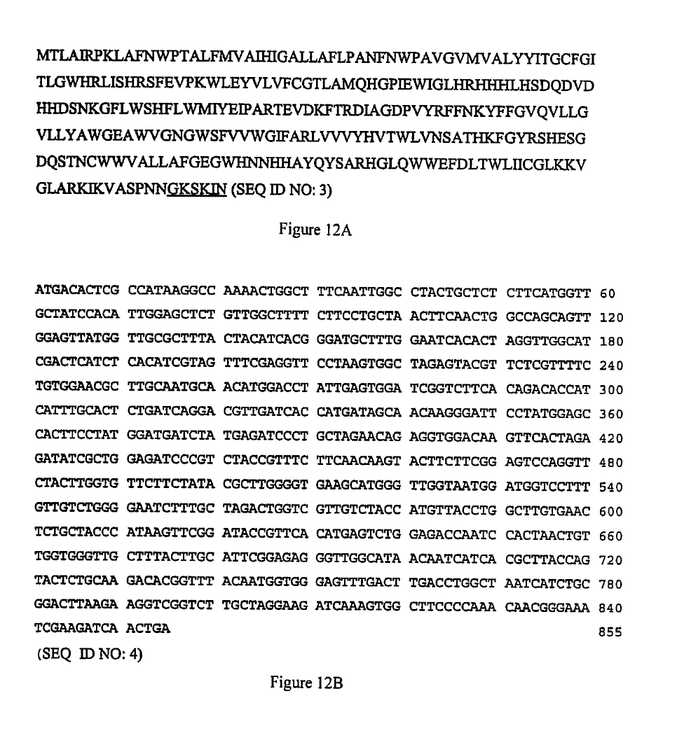

When we transformed Arabidopsis with seed-specific expression constructs encoding DES9*Q240R and DES9*(E69R, Q240R), we discovered that the mutant proteins were in fact much more active than the original DSG or DES9 proteins. Most importantly, DES9* were effective in reducing the level of 16:0 and 18:0 in Arabidopsis seed; these changes were maintained in succeeding generations of seeds. For both 16C and 18C substrates, the monounsaturated products of the desaturation, 16:1 and 18:1, were greatly increased (Table 9). When individual transgenic lines were examined (FIG. 8), some DES9* lines had very low levels of 16:0, and detailed analysis of these lines clearly indicates that DES9*Q240R had a much greater ability to reduce the 16:0 found in Arabidopsis seed (FIG. 10). These results show that the experimental approach of directed evolution of a prokaryotic desaturarse enzyme in yeast, selecting for increased activity, followed by expression in Arabidopsis led to successful modification of the content of oilseeds.

The success we have demonstrated here opens the door to modifying other prokaryotic enzymes, either from cyanobacteria or other bacteria. A wide variety of cyanobacterial desaturases are known (Chi et al., 2008). Directed evolution in the ole1-mutant yeast background is especially useful in finding variants that are more active desaturases, since ole1 can be complemented by a range of monounsaturated and polyunsaturated fatty acids.

In summary, because desaturation activity of cyanobacterial glycerolipid delta-9 desaturase expressed in Arabidopsis seeds was low, we used a series of strategies to improve desaturation activity, including codon optimization, fusion of an ER retention signal peptide, and finally by application of directed evolution. The directed evolution experiments used standard mutation techniques applied through a strategy of complementing the growth phenotype of a yeast desaturase mutant to screen for increased desaturase activity. We discovered that certain single amino acid changes, or combined changes of several amino acids, could dramatically improve desaturation activity in yeast. By transforming the mutagenized desaturases into Arabidopsis under control of a seed specific promoter, we validated the directed evolution approach, because mutant proteins with higher fatty acid conversion in yeast were likewise more effective in reducing saturates in Arabidopsis seeds. We not only increased levels of 16:1 and 18:1 fatty acids, but importantly achieved a long-term goal of plant fatty acid metabolic engineering, reducing the proportion of the 16:0 and 18:0 saturated fatty acids in oilseeds to a small fraction of that found in non-transgenic controls.

### Example 2

### Application of a Cyanobacterial Desaturase to Reduce Saturated Fatty Acids in Camelina Seed Crop

### Experimental Procedures

### Camelina Transformation

Camelina sativa was grown in a greenhouse with 16-h day (21-24° C.) and 8-h dark (17−20° C.), at 50% humidity, and with natural lighting supplemented to maintain at least 250 μmol m⁻² s⁻¹ during the day. Camelina transformation followed the protocol of Lu and Kang, (2008). The early flowers of intact Camelina were immersed in a prepared Agrobacterium suspension within a vacuum chamber, and the pressure reduced to about 85 kPa for 5 min, after which the plants were allowed to recover.

### Seed Fatty Acid Analysis

For Camelina seed oil analysis, six seeds in a glass vial were crushed with a glass rod, then fatty acid methyl esters prepared and analyzed as described for Arabidopsis seed above. Seed weight and Germination

To characterize the phenotype of the Camelina seeds, we measured average weight by counting approximately 50 red seed and about 50 brown seed, weighing the samples, and dividing the weight by the number of seeds. For germination assays, we sterilized approximately 20 red Camelina seed from each line, distributed them on (MS+sucrose) agar plates and scored them for germination after five days incubation under continuous light; emergence of green tissue was scored as germination.

### Results

Camelina Seeds Expressing Des9* have Reduced 16:0 and 18:0, and Higher Levels of 16:1 and 18:1

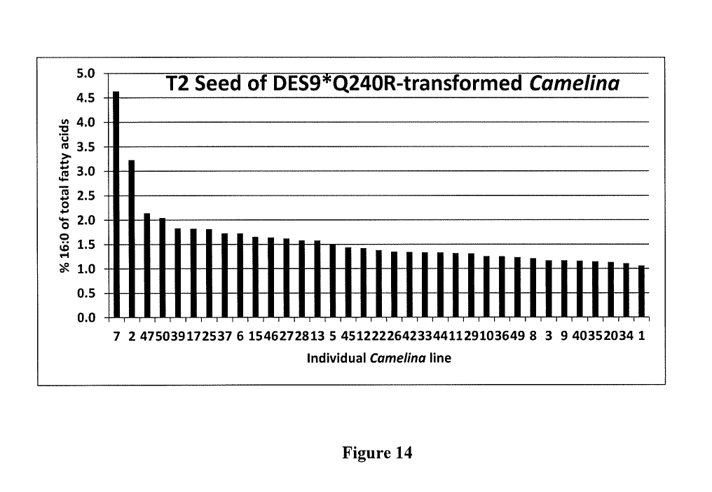

To test how DES9* variants perform in an oilseed crop, we transformed Camelina sativa using the same constructs used successfully to transform Arabidopsis (see above), including vectors expressing DES9, DES9*Q240R, DES9*23, DES9*24 or DES9*26 variants under control of the phaseolin seed-specific promoter. Transgenic seed could be separated from untransformed ones by visual selection based on expression from within the T-DNA insert of the dsRed marker. Transformed red seed from each experiment were sorted from the untransformed brown seed. We chose more than 20 red T1 seeds at random to plant, and when these progeny set seed we analyzed the fatty acid composition of the resulting T2 seed, again choosing red seed for analysis. The levels of 16:0 in the T2 seed of the 21 DES9*Q240R lines varied from 4.7 to 1.2% of the total (FIG. 14), whereas brown, untransformed seed had on average 6.7% of their total fatty acid as 16:0.

Three lines transformed with the construct DES9*Q240R that had very low 16:0 levels were selected for further analysis. As shown in Table 10, the level of 16:0 in these lines is reduced to between 1.2% and 1.4% in red transgenic T2 seeds, an 80% reduction compared to untransformed brown seeds. When we analyzed seed of the succeeding generation, the 16:0 found in T3 homozygous seeds was 1.2% for all three lines. When we assessed germination rate for these low 16:0 lines it was equivalent to that of untransformed seed (Table 10).

TABLE 10
Expression of DES9*Q240R reduces the level of 16:0 in Camelina
seeds. The levels of 16:0, seed weight, and germination in T2,
and 16:0 level of T3, red transgenic or brown untransformed Camelina
seeds. Data represent means of 6 determinations, ± S.D.

| | | T2 | | | T3 |
|---|---|---|---|---|---|
| | Transgenic red or | 16:0% | | Germination | 16:0% |
| Line | WT brown | (n = 6) | mg/seed | (%) | (n = 6) |
| 35 | Brown | 6.7 ± 0.2 | 1.39 | 100 | 6.7 ± 0.3 |
| | Red | 1.2 ± 0.1 | 1.19 | 100 | 1.2 ± 0.1 |
| 40 | Brown | 6.9 ± 0.2 | 1.39 | 100 | 6.6 ± 0.3 |
| | Red | 1.4 ± 0.1 | 1.12 | 95 | 1.2 ± 0.1 |
| 1 | Brown | 6.7 ± 0.2 | 1.31 | 100 | 6.7 ± 0.2 |
| | Red | 1.4 ± 0.1 | 1.10 | 87.5 | 1.2 ± 0.2 |

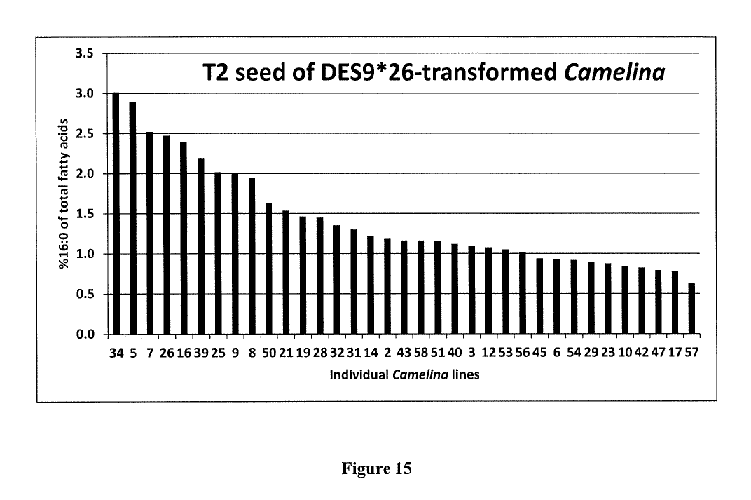

We measured the levels of 16:0 found in T2 red seeds from T1 plants separately transformed with constructs expressing DES9, DES9*Q240R, DES9*23, DES9*24 or DES9*26. Expression of each DES9* desaturase reduced the level of 16:0 and 18:0 in the Camelina seed, while the levels of 16:1 and 18:1 increased. When we examined the lowest level of 16:0 measured after transformation with each construct (Table 11), the effect on 16:0 and 18:0 was different amongst the DES9* variants, but each one had much greater effect on the level of saturated fatty acids than the parent DES9 (Table 11). Transformation with DES9*26 produced 10 of 35 lines with lower 16:0 levels than the best DES9*Q240R lines observed in T2 red seeds (FIG. 15 and Table 11). In the best DES9*26 line, the 16:0 in T2 red seed has been reduced to 0.6%, a 90% reduction from the 6.7% 16:0 of wild-type Camelina. The effect on 18:0 was also pronounced; the 2.4% level of 18:0 found in wild-type was reduced to one-sixth of that value, 0.4% for the DES9*26-transformed line. The level of 18:1 increased from the wild-type level of 9.8% to 17.5% in that same line, an increase of about 1.8-fold. Saturated 16:0 and 18:0 make up 9.1% of the total fatty acids in these wild-type Camelina seed, but only 1% remains in the DES9*26 seeds, an 89% reduction. Further reductions in saturated fatty acids and increases in 16:1 and 18:1 levels in Camelina seeds are expected in these types of lines.

TABLE 11
Expression of DES9* variants reduces the levels of 16:0 and
18:0 in Camelina seeds. Levels of 16C and 18C change in red
transgenic Camelina seeds. Data represent analyses of
six red T2 seed from lines with the lowest 16:0 derived from
each transformation construct so far examined.

| | | | | | Reduction in |
|---|---|---|---|---|---|
| | | | | | saturates (% of |
| Transgene | 16:0% | 16:1% | 18:0% | 18:1% | wild-type) |
| WT | 6.7 | 0.2 | 2.4 | 9.8 | 0 |
| DES9 | 3.1 | 5.6 | 1.5 | 13.9 | 49.5 |
| DES9* Q240R | 1.1 | 8.9 | 0.8 | 14.3 | 79.1 |
| DES9* 23 | 1.2 | 8.5 | 0.8 | 15 | 78.0 |
| DES9*24 | 1 | 8.9 | 0.8 | 14.7 | 80.2 |
| DES9*26 | 0.6 | 7.7 | 0.4 | 17.5 | 89.0 |

### Discussion

Successful reduction of the level of 16:0 fatty acid by expression of DES9 variants in Arabidopsis, as described above, led us to express some of the same variant proteins in Camelina sativa, a plant cultivated for its oil in Europe and North America. We employed the DNA vector constructs and Agrobacterium strains that had proved successful in Arabidopsis, employing a whole-plant transformation protocol from the recent literature (Lu and Kang 2008), and relying on expression of dsRed as a marker for transformed seed. Our first transformation used a construct expressing DES9*Q240R, a variant which had been very successful in reducing 16:0 levels in Arabidopsis. The T1 red transformed Camelina seed we planted produced plants whose T2 red seed all exhibited low levels of 16:0, ranging from just over 4.5% 16:0 to as low as 1.2% 16:0 (Figure AD1), compared to the parental level of 6.7%. We chose three lines for further investigation, and after planting the T2 seed, we found that the T3 seed of the progeny plants maintained their low 16:0 phenotype (Table AD1), showing both that Camelina with low 16:0 are viable and that the low-16:0 phenotype induced by the transgenic DES9* variant is heritable.

Since some variants of DES9* had more desaturase activity in yeast than DES9*Q240R, we elected to transform Camelina with other selected variants. Transformation using constructs expressing each DES9* variant tested yielded plant lines whose T2 seed displayed a range of 16:0 levels, invariably lower than the parental 16:0 concentration. For example, T2 seed resulting from transformation with DES9*26 had levels of 16:0 ranging from 3% to as low as 0.6% (FIG. 15). DES9*26 expression results in the conversion of more than 90% of the 16:0 fatty acids in the seed to 16:1, while converting all but 0.4% of the 18:0 to 18:1 (Table 11). We conclude that expression of DES9* variants modified the fatty acid constituents of the oilseed crop Camelina, reducing the 16:0 and 18:0 fatty acids to a small fraction of the parental levels while elevating the levels of 16:1 and 18:1.

By transforming the mutagenized desaturases Camelina under control of a seed-specific promoter, we showed that we could reduce saturates in seeds of an oilseed crop plant. The reductions achieved in Camelina were even more pronounced than those measured in Arabidopsis. Expression of DES9* desaturases reduced the proportion of 16:0 and 18:0 saturated fatty acids to a small fraction of that found in non-transgenic controls and significantly increased levels of 16:1 and 18:1 fatty acids.

### REFERENCES

Bendtsen J. D., Nielsen H., von Heijne G., and Brunak, S. (2004). Improved prediction of signal peptides: SignalP 3.0. Journal of Molecular Biology 340, 783-795.
Bonaventure, G., Salas, J. J., Pollard, M. R., and Ohlrogge, J. B. (2003). Disruption of the FATB gene in Arabidopsis demonstrates an essential role of saturated fatty acids in plant growth. The Plant cell 15, 1020-1033.
Bondaruk, M., Johnson, S., Degafu, A., Boora, P., Bilodeau, P., Morris, J., Wiehler, W., Foroud, N., Weselake, R., and Shah, S. (2007). Expression of a cDNA encoding palmitoyl acyl carrier protein desaturase from cat's claw (Doxantha unguis cati L.) in Arabidopsis thaliana and Brassica napus leads to accumulation of unusual unsaturated fatty acids and increased stearic acid content in the seed oil. Plant breeding 126, 186-194.
Chi, X. Y., Yang, Q. L., Zhao, F. Q., Qin, S., Yang, Y., Shen, J. J., and Lin, H. Z. (2008). Comparative Analysis of Fatty Acid Desaturases in Cyanobacterial Genomes. Comparative and Functional Genomics.
Clough, S. J., and Bent, A. F. (1998). Floral dip: a simplified method for Agrobacterium-mediated transformation of Arabidopsis thaliana. Plant Journal 16, 735-743.
Craig, W., Lenzi, P., Scotti, N., De Palma, M., Saggese, P., Carbone, V., McGrath Curran, N., Magee, A. M., Medgyesy, P., Kavanagh, T. A., Dix, P. J., Grillo, S., and Cardi, T. (2008). Transplastomic tobacco plants expressing a fatty acid desaturase gene exhibit altered fatty acid profiles and improved cold tolerance. Transgenic research 17, 769-782.
Fahy, D., Scheer, B., Wallis, J. G., and Browse, J. (2013). Reducing saturated fatty acids in Arabidopsis seeds by expression of a Caenorhabditis elegans 16:0-specific desaturase. Plant biotechnology journal 11, 480-489.
Krogh, A., Larsson, B., von Heijne, G., and Sonnhammer, E. L. L. (2001). Predicting transmembrane protein topology with a hidden Markov model: Application to complete genomes. Journal of Molecular Biology 305, 567-580.
Lu, C., Fulda, M., Wallis, J. G., and Browse, J. (2006). A high-throughput screen for genes from castor that boost hydroxy fatty acid accumulation in seed oils of transgenic Arabidopsis. The Plant journal: for cell and molecular biology 45, 847-856.
Lu C, Kang J (2008) Generation of transgenic plants of a potential oilseed crop Camelina sativa by Agrobacterium-mediated transformation. Plant cell reports 27: 273-278
McCartney, A. W., Dyer, J. M., Dhanoa, P. K., Kim, P. K., Andrews, D. W., McNew, J. A., and Mullen, R. T. (2004). Membrane-bound fatty acid desaturases are inserted co-translationally into the ER and contain different ER retrieval motifs at their carboxy termini. Plant Journal 37, 156-173.
Miguel, M., and Browse, J. (1992). Arabidopsis Mutants Deficient in Polyunsaturated Fatty-Acid Synthesis—Biochemical and Genetic-Characterization of a Plant Oleoyl-Phosphatidylcholine Desaturase. Journal of Biological Chemistry 267, 1502-1509.
Moon, H., Hazebroek, J., and Hildebrand, D. F. (2000). Changes in fatty acid composition in plant tissues expressing a mammalian delta9 desaturase. Lipids 35, 471-479.
Nguyen, H. T., Mishra, G., Whittle, E., Pidkowich, M. S., Bevan, S. A., Merlo, A. O., Walsh, T. A., and Shanklin, J. (2010). Metabolic engineering of seeds can achieve levels of omega-7 fatty acids comparable with the highest levels found in natural plant sources. Plant physiology 154, 1897-1904.
Polashock, J. J., Chin, C. K., and Martin, C. E. (1992). Expression of the Yeast Delta-9 Fatty Acid Desaturase in Nicotiana tabacum. Plant physiology 100, 894-901.
Smith, M. A., Dauk, M., Ramadan, H., Yang, H., Seamons, L. E., Haslam, R. P., Beaudoin, F., Ramirez-Erosa, I., and Forseille, L. (2013). Involvement of Arabidopsis ACYL-COENZYME A DESATURASE-LIKE2 (At2g31360) in the Biosynthesis of the Very-Long-Chain Monounsaturated Fatty Acid Components of Membrane Lipids. Plant physiology 161, 81-96.
Stuitje, A. R., Verbree, E. C., van der Linden, K. H., Mietkiewska, E. M., Nap, J. P., and Kneppers, T. J. (2003). Seed-expressed fluorescent proteins as versatile tools for easy (co)transformation and high-throughput functional genomics in Arabidopsis. Plant biotechnology journal 1, 301-309.
Stukey, J. E., McDonough, V. M., and Martin, C. E. (1989). Isolation and Characterization of Ole1, a Gene Affecting Fatty-Acid Desaturation from Saccharomyces-Cerevisiae. J Biol Chem 264, 16537-16544.
Wada, H., Schmidt, H., Heinz, E., and Murata, N. (1993). Invitro Ferredoxin-Dependent Desaturation of Fatty-Acids in Cyanobacterial Thylakoid Membranes. J Bacteriol 175, 544-547.
Yao, K., Bacchetto, R. G., Lockhart, K. M., Friesen, L. J., Potts, D. A., Covello, P. S., and Taylor, D. C. (2003). Expression of the Arabidopsis ADS1 gene in Brassica juncea results in a decreased level of total saturated fatty acids. Plant Biotechnology Journal 1, 221-229.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of modulating fatty acid content or composition profile in a eukaryotic host, comprising
genetically engineering said eukaryotic host so as to contain and express a DNA molecule encoding a recombinant prokaryotic desaturase,
wherein desaturase activity of said recombinant prokaryotic desaturase is at least 1.5 fold higher than that of a corresponding native prokaryotic desaturase,
wherein at least one of a level of one or more saturated fatty acids is decreased or a level of one or more unsaturated fatty acids is increased in said eukaryotic host or both.

2. The method of claim 1,
wherein said recombinant prokaryotic desaturase is a genetically engineered mutant form of a cyanobacterium desaturase.

3. The method of claim 2,
wherein said cyanobacterium desaturase is glycerolipid Δ9-desaturase (DSG) from Synechococcus elongatus (Anacystis nidulans, PCC 6301).

4. The method of claim 3, wherein an amino acid sequence of said recombinant prokaryotic desaturase includes a mutation at one or more of the positions listed in Table 2 or one or more mutations or combinations of mutations listed in Table 3.

5. The method of claim 4, wherein said one or more of the mutations includes Arg or Lys at one or both of positions 69 and 240.

6. The method of claim 4, wherein said recombinant prokaryotic desaturase includes a combination of substitutions of Arg at position 132, Gly at position 214 and Gln at position 240 or a combination of Lys at position 8, His at position 88, Arg at position 132 and Gln at position RECTIFIED SHEET (RULE 91) 240 or a combination of Glu at position 69, Ile at position 129, Leu at position 225 and Gln at position 240.

7. The method of claim 1, wherein said recombinant prokaryotic desaturase comprises one or more eukaryotic sequences.

8. The method of claim 7, wherein said one or more eukaryotic sequences include an endoplasmic reticulum retention sequence.

9. The method of claim 1, wherein said eukaryotic host is selected from the group of oil seed plants, canola, safflower, camelina, soybean, corn, sunflower, peanut, sesame, cotton rice, wheat, coconut, cotton, olive, palm, peanut, flax, hazelnuts, almond, beech, cashew, macadamia, mongongo, pecan, pine, pistachio, walnut, lemon, orange oil, grapefruit, sea-buckthorn, watermelon, gourds, melons, pumpkins, squashes, butternut squash, egusi, borage, blackcurrant, evening primrose, acai, black seed, blackcurrant, flax, carob, amaranth, apricot, apple, argan, avocado, babassu, cape chestnut, the cacao plant, cocklebur, poppy, cohune palm, coriander, date, Irvingia gabonensis, Camelina sativa, grape, hemp, Ceiba pentandra, Hibiscus cannabinus, lallemantia iberica, Trichilia emetica, Sclerocarya birrea, meadowfoam, mustard, nutmeg, okra, papaya, perilla, persimmon, Caryocar brasiliense, pili nut, pomegranate, prune quinoa, ramtil, Niger pea, rice Prinsepia utilis, shea, Sacha inchi, sapote, arugula, tea (Camellia), thistle, Cyperus esculentus, tobacco, tomato, and Brassica oilseed plants including Brassica juncea, Brassica napus, Brassica carinata, Brassica nigra, Brassica rapa or Brassica campestris.

10. The method of claim 1, wherein said eukaryotic host is Arabidopsis thaliana or Camelina saliva.

11. A nucleic acid molecule comprising nucleotide sequences which encode and express a recombinant prokaryotic desaturase,
wherein desaturase activity of said recombinant prokaryotic desaturase is at least 1.5 fold higher than that of a corresponding native prokaryotic desaturase.

12. The nucleic acid molecule of claim 11, wherein said recombinant prokaryotic desaturase is a genetically engineered mutant form of a cyanobacterium desaturase.

13. The nucleic acid molecule of claim 12, wherein said cyanobacterium desaturase is glycerolipid Δ9-desaturase (DSG) from Synechococcus elongatus (Anacystis nidulans, PCC 6301).

14. The nucleic acid molecule of claim 13, wherein an amino acid sequence of said recombinant prokaryotic desaturase includes a mutation at one or more of the positions listed in Table 2 or one or more mutations or combinations of mutations listed in Table 3.

15. The nucleic acid molecule of claim 14, wherein said one or more of the mutations includes Arg or Lys at one or both of positions 69 and 240.

16. The nucleic acid molecule of claim 14, wherein said recombinant prokaryotic desaturase includes a combination of substitutions of Arg at position 132, Gly at position 214 and Gln at position 240 or a combination of Lys at position 8, His at position 88, Arg at position 132 and Gln at position 240 or a combination of Glu at position 69, Ile at position 129, Leu at position 225 and Gln at position 240.

17. The nucleic acid molecule of claim 11, wherein i) said nucleic acid molecule is codon optimized for expression in a eukaryotic host; and/or ii) AT/CG ratios of said nucleic acid molecule are modified for expression in a eukaryotic host.

18. A transgenic eukaryotic host which is genetically engineered to contain and express a nucleic acid molecule that encodes a recombinant prokaryotic desaturase, wherein desaturase activity of said recombinant prokaryotic desaturase is at least 1.5 fold higher than that of a corresponding native prokaryotic desaturase.

19. The transgenic eukaryotic host of claim 18, wherein said recombinant prokaryotic desaturase is a genetically engineered mutant form of a cyanobacterium desaturase.

20. The transgenic eukaryotic host of claim 19, wherein said cyanobacterium desaturase is glycerolipid Δ9-desaturase (DSG) from Synechococcus elongatus (Anacystis nidulans, PCC 6301).

21. The transgenic eukaryotic host of claim 20, wherein an amino acid sequence of said recombinant prokaryotic desaturase includes a mutation at one or more of the mutations listed in Table 2 or one or more mutations or combinations of mutations listed in Table 3.

22. The transgenic eukaryotic host of claim 21, wherein said one or more of the mutations includes Arg or Lys at one or both of positions 69 and 240.

23. The transgenic eukaryotic host of claim 18, wherein said recombinant prokaryotic desaturase includes a combination of substitutions of Arg at position 132, Gly at position 214 and Gln at position 240 or a combination of Lys at position 8, His at position 88, Arg at position 132 and GM at position 240 or a combination of Glu at position 69, Ile at position 129, Leu at position 225 and Gln at position 240.

24. The transgenic eukaryotic host of claim 18, wherein said nucleic acid molecule is codon optimized for expression in a eukaryotic host.

25. The transgenic eukaryotic host of claim 18, wherein said transgenic eukaryotic host is a plant or plant cell; an animal or an animal cell; or a fungal cell.

26. The transgenic eukaryotic host of claim 25, wherein said transgenic eukaryotic host is a plant and said recombinant prokaryotic desaturase further comprises an endoplasmic reticulum retention sequence.

27. The transgenic eukaryotic host of claim 26, wherein said plant is an oil seed producing plant.

28. A product produced by or from a transgenic eukaryotic host which is genetically engineered to contain an express a nucleic acid molecule that encodes a recombinant prokaryotic desaturase, wherein desaturase activity of said recombinant prokaryotic desaturase is at least 1.5 fold higher than that of a corresponding native prokaryotic desaturase.
